(19) United States
(12) Patent Application Publication
(10) Pub. No.: US 2007/0082350 A1
(43) Pub. Date: Apr. 12, 2007

(54) Assay and method for diagnosing and treating alzheimer's disease

(76) Inventors: Philip Landfield, Lexington, KY (US); Nada Porter, Lexington, KY (US); Kuey-Chu Chen, Lexington, KY (US); James Geddes, Lexington, KY (US); Eric Blalock, Lexington, KY (US)

Correspondence Address: MCDERMOTT WILL & EMERY LLP, 600 13TH STREET, N.W., WASHINGTON, DC, 20005-3096, US

(21) Appl. No.: 11/501,226

(22) Filed: Aug. 9, 2006

(30) Foreign Application Priority Data
Feb. 9, 2005 (WO) PCT/US05/03668

(51) Int. Cl. C12Q 1/68; C12M 3/00

(52) U.S. Cl. 435/6; 435/287.2

(57) ABSTRACT

I Methods and kits for diagnosing Alzheimer's disease and/or incipient Alzheimer's disease are disclosed. The methods and kits of the invention utilize a set of genes and their encoded proteins that are shown to be correlated with incipient Alzheimer's disease.

### RELATED APPLICATIONS

This application claims priority from PCT Application No. PCT/US2005/003668, filed on Feb. 9, 2005 which in turn claims priority to provisional application Ser. No. 60/542,281, filed Feb. 9, 2004, incorporated herein in its entirety.

### FIELD OF THE INVENTION

The invention relates to assays and methods for diagnosing and treating Alzheimer's disease (AD). More particularly, this invention relates to methods for detecting changes in the pattern of gene expression that correlated with AD, and in particular, with incipient AD, and using these changes to either diagnose AD in a patient or screen compounds for treating AD.

### BACKGROUND OF THE INVENTION

Alzheimer's disease (AD) has received intense study during the past decades. Multiple processes have been implicated in AD, notably including abnormal beta-amyloid production, tau hyperphosphorylation and neurofibrillary tangles (NFTs), synaptic pathology, oxidative stress, inflammation, protein processing or misfolding, calcium dyshomeostasis, aberrant reentry of neurons into the cell cycle, cholesterol synthesis, and effects of hormones or growth factors. Nevertheless, the pathogenic factors that initiate these processes remain elusive.

Several reasons account for the substantial resistance of AD pathogenesis to analysis. One is the vast extent and complexity of the disease, which affects numerous molecules, cells, and biochemical pathways. Another is that clinically normal subjects may exhibit considerable AD pathology, blurring criteria for distinguishing subjects with normal aging, mild cognitive impairment, or incipient AD or progressive AD.

Thus, there is a need for an assay that enables the medical practitioner to distinguish these conditions.

### SUMMARY OF THE INVENTION

In one aspect of the invention there is provided an oligonucleotide or cDNA array comprising a solid support comprising a plurality of different oligonucleotide probes or cDNA probes, each oligonucleotide probe or cDNA specific for a gene listed in Table 6, Table 5 or Table 4. In another aspect of the invention there is provided a kit comprising an oligonucleotide array of the invention and a reagent. In another aspect there is provided an array with a plurality of probes for measuring proteins encoded by the genes listed in Tables 6, 5 or 4.

In another aspect of the invention there is provided a method of detecting in a body sample of a patient or experimental subject a reliable alteration in the expression pattern of at least one gene or a profile of genes correlated with incipient Alzheimer's disease (IAD) or Alzheimer's disease (AD) relative to expression of said profile or at least one gene in a pooled or individual control sample. The method comprises a) obtaining RNA from said body sample; b) contacting said RNA with an array according to claim 1 under conditions that permit hybridization of said RNA to oligonucleotide covalently attached to said array; and c) detecting the presence or absence of said alteration in the expression pattern of at least one gene correlated with incipient Alzheimer's disease (IAD) or Alzheimer's disease (AD) relative to expression of said at least one gene in a control. In preferred embodiments, the body sample may be a brain sample or neural tissue sample.

In a further aspect of the invention there is provided a method for diagnosing IAD in a patient. The method comprises a) obtaining a body sample from the patient and extracting RNA there from; b) contacting said RNA with an array according to claim 1 under conditions that permit hybridization of said RNA to oligonucleotide covalently attached to said array; c) detecting the presence or absence of an alteration in the expression pattern of at least one gene correlated with incipient Alzheimer's disease (IAD) relative to expression of said at least one gene in a control; and d) using the presence of an alteration in the expression pattern of at least one gene correlated with incipient Alzheimer's disease (IAD) relative to expression of said at least one gene in a control to diagnose the presence of IAD. In another embodiment, the method further comprises administering MMSE or other neuropyschological test to the patient.

In yet a further aspect of the invention, there is provided a method of screening a test compound for treatment of AD or IAD. The method comprises a) administering the test compound to an animal or human exhibiting all or some of the symptoms of AD; b) obtaining a body sample from the animal and obtaining RNA there from; c) contacting said RNA with an array according to claim 1 under conditions that permit hybridization of said RNA to oligonucleotides covalently attached to said array; and c) detecting the presence or absence of an alteration in the expression pattern of at least one gene listed in Table 6 relative to expression of said gene in an untreated control animal or human exhibiting all or some of the symptoms of AD.

In another aspect of the invention there is provided a method of detecting an alteration in the expression pattern of a plurality of genes correlated with Alzheimer's disease relative to expression of said plurality of genes in a control in a brain tissue or neural tissue sample of an animal, said method comprising a) measuring the relative amount of individual proteins in said sample, wherein each of said proteins is encoded by a gene correlated with IAD; and b) correlating an increase or decrease in the amount of a plurality of said proteins relative to amount of said plurality of proteins to an alteration in the expression pattern of the plurality of genes encoding said proteins. In preferred embodiments, the IAD associated genes are selected from those listed in Tables 6 or 4.

### BRIEF DESCRIPTION OF THE DRAWINGS

FIG. 1 is a cartoon of a gene identification algorithm. (A) Genes rated absent were excluded from analysis. (B) Only annotated probe sets (not expressed sequence tags) were included in the statistical analysis. (C) Pearson correlation was performed for every gene against both MMSE and NFT measures of each subject. Venn diagram shows the number of genes significantly correlated (P≦0.05) with both MMSE and NFT or either index alone. For each index the false discovery rate (FDR) was calculated. (D) For the genes found to correlate significantly across all subjects (overall, n=31), another Pearson's correlation was performed post hoc among only the subjects rated “Control” or “Incipient” (Incipient, n=16).

FIG. 2. Graphs of examples of correlated genes illustrating the four directions of correlation through which genes were identified. For each gene, expression density is plotted on the y axis, and MMSE (A left and C left) or NFT (B right and D right) scores are plotted on the x axis; R² value, P value (Pearson's test), linear fit (black line), and 95% confidence intervals (dashed lines) are also shown. (A and B) Genes for which expression levels were up-regulated with AD, identified with negative or positive correlation with MMSE (A) or NFT (B) scores, respectively. (C and D) Genes for which expression levels were down-regulated with AD, identified by negative or positive correlation with MMSE (C) or NFT (D), respectively. The MMSE scale is reversed, so that more advanced AD increases to the right on both indexes.

FIG. 3. Table 5. A list of AD-correlated genes, probes and data showing correlation to AD, IAD, NFT, and/or MMSE. Alzheimer's disease genes (ADGs) are listed in alphabetical order by gene name for up-regulated (positively correlated with NFT_{O} and/or negatively correlated with MMSE_{O}) and down-regulated (negatively correlated with NFT_{O}, and/or positively correlated with MMSE_{O}) categories. Description, gene title from Affymetrix annotation database. NFT_{O} and MMSE_{O}, overall Pearson's correlations with neurofibrillary tangle (NFT-former) and Mini-Mental Status Exam (MMSE-latter) for all 31 subjects. NFT₁ and MMSE₁, correlations across only control and incipient subjects (n=16). Negative correlations have negative P values. ANOVA, P value for one-way ANOVA tests across the following groups: control, incipient, moderate, and severe. Gene expression data for each group are mean±SEM

FIG. 4. Table 6. A list of IAD-correlated genes, probes and data showing correlation to IAD. Incipient Alzheimer's disease genes (IADGs) are listed in alphabetical order by gene name for up-regulated (positively correlated with NFT₁ and/or negatively correlated with MMSE₁) and down-regulated (negatively correlated with NFT₁, and/or positively correlated with MMSE₁) categories. Description, gene title from Affymetrix annotation database are provided. NFT_{O} and MMSE_{O}, overall Pearson's correlations with neurofibrillary tangle (NFT-former) and Mini-Mental Status Exam (MMSE-latter) for all 31 subjects. NFT₁ and MMSE₁, correlations across only control and incipient subjects (n=16). Negative correlations have negative P values. ANOVA, P value for one-way ANOVA tests across the following groups: control, incipient, moderate, and severe. Gene expression data for each group are mean±SEM.

### DETAILED DESCRIPTION OF THE INVENTION

The present inventors addressed the problems of high complexity and overlapping criteria for diagnosing incipient AD (IAD) by using a strategy that combines a powerful new gene microarray technology, which permits measurement of the expression of many thousands of genes simultaneously, with statistical correlation analysis. This strategy has allowed the linking of gene expression to cognitive and pathological markers independent of AD diagnosis and has led to the identification of genes correlating with AD and in particular, IAD.

Several microarray studies of AD brain and/or mouse models of AD have been published. For example, U.S. Pat. No. 6,838,592 discloses a mouse model for Alzheimer's disease, and U.S. Pat. No. 6,852,497 discloses a transgenic mouse for testing compounds useful for AD. The microarray studies have yielded important new insights, in particular, regarding changes in plasticity-related genes (e.g., Dickey et al., (2003) J. Neurosci., 23:5219-5226). However, few microarray studies use independent sample sizes to provide the statistical power needed to avoid high false positive (type I) and/or high false negative (type II) error (Miller et al., (2001) J. Gerontol. A Biol. Sci. Med. Sci., 56:B52-B57; Blalock et al., (2003) J. Neurosci., 23:3807-3819). In contrast to these applications, in the development of the present invention, adequate power was ensured by using a separate array for each hippocampal sample of a large group of subjects (n=31) and correlating the expression values of each of thousands of genes with pathological and cognitive indexes of incipient AD. The subjects in the AD study were assigned to four groups reflecting different levels of AD severity (incipient, moderate or severe) or control (Table 1), but the correlation analyses were independent of this initial diagnosis. Together, these approaches represent the first formal statistical correlation analysis between pathological markers of AD and thousands of genes on a microarray. The set of correlated genes therefore comprises a unique and valuable set of genes that, together or in small subsets, can be used to diagnose AD with greater accuracy than has been possible. Further, because these analyses revealed a major and previously unrecognized transcriptional response with important implications for the early pathogenesis of AD, these lists of correlated genes can be used to screen and develop new compounds for the treatment of AD.

Based on these large-scale studies, a list of genes that correlate with Alzheimer's disease (ADGs) that appear to have considerable potential importance for assessing AD and IAD and generating new treatments for AD has been generated (TABLES 5 and 6). These lists contain some genes, or proteins encoded by said genes, that were identified previously as being linked to AD (e.g., inflammation-related genes) but none has been previously shown to be formally correlated with IAD. Further, many genes on the lists have not even been shown previously to be linked to AD or IAD. Thus, the lists of Alzheimer's disease-related genes (ADGs) or incipient-correlated ADGs (IADGs) are unique and useful biomarkers and therapeutic targets specifically for AD and/or IAD. In addition, the list of all genes whose expression pattern changes with IAD and/or AD contains many genes never before reported to change with AD or IAD, and therefore provides a useful and unique panel of gene biomarkers and therapeutic targets for study and treatment of AD.

Using the method of the invention, a number of processes and pathways that previously have not been clearly associated with AD have been identified. The present inventors have discovered that widespread changes in genomic regulation of multiple cellular pathways are major correlates of incipient AD and hence, further developed AD. As noted, it has been recognized previously that inflammation, synaptic dysfunction, energy failure, glial reactivity, protein misprocessing or misfolding, lipogenesis and cell cycle disturbances accompany AD. However, the main transcriptional orchestration seen in incipient AD may provide a new perspective on the possible origins of these deleterious processes, and provide new targets for therapy. In addition, the widespread activation of growth, differentiation, and tumor suppressor (TS) pathways, and the apparent collapse of protein-processing machinery so early in the disease, suggest clues to the early pathogenesis of AD. The detection of these process patterns also provides a diagnostic tool for incipient AD and more progressive AD. These conclusions are supported by high levels of statistical confidence for individual genes and by statistical evidence of co-regulation of genes within related pathways and categories (Tables 2 and 3).

Multiple tumor suppressors (TSs), some of which regulate the cell cycle, were identified using the present method within the TF (Table 4) and other categories. Previous studies have found evidence of cell cycle reentry in neurons of the AD brain (Arendt et al., (2000) Ann. N.Y. Acad. Sci., 920:249-255; Bowser et al. (2002) J. Alzheimer's Dis. 4:249-254), and a handful of studies have also examined TSs in relation to AD, largely in terms of their roles in apoptotic pathways (e.g., p 52) (See Bowser et al., supra). However, TSs have other actions unrelated to apoptosis and can, in fact, be antiapoptotic (See Slack et al. (1995) J. Cell Biol., 129:779-788). Notably, TSs play critical roles in cellular differentiation related to development and tumor suppression. For example, overexpression of some TSs (e.g., RB proteins) induces cell cycle arrest, differentiation, and process extension in astrocytomas (See Galderisi et al. (Mol. Cell. Neurosci., 17:415-425). TS expression also is necessary for neurite extension and synaptogenesis in neuronal development (See Slack et al., supra). Moreover, in some cell types, TSs operate by inducing cellular senescence and inhibiting protein biosynthesis (Campisi, J. (2001) Trends Cell Biol., 11:S27-S31).

TSs can be activated by developmental factors, DNA/cellular damage, or dysregulation of the cell cycle. Therefore, oxidative stress, inflammation, or abnormal CA²⁺ signaling are clearly candidate activators of TSs. In addition, TSs act as negative feedback regulators of growth and are often elevated in response to excess growth factor (GF) production in tumors (73). Many unregulated DGs also were identified here (Table 4), perhaps originating in OGs and their progenitors, which retain substantial growth potential in adult brain. Consistent with this possibility, several of the up regulated IADGs, including PDGFB, FYN, and FGFR3, play major roles in OG proliferation, differentiation, and myelinogenesis.

The present studies have revealed widespread and apparently orchestrated transcriptional responses associated with early signs of AD pathology. Dissecting the bases for these early responses should yield important insights into pathogenic mechanisms and suggest therapeutic approaches to AD. Further, by testing for changes in the pattern of gene expression of the genes shown herein to be correlated with AD or IAD, or subsets of these genes, an accurate diagnosis of AD or IAD can be made. Gene expression patterns for these genes, and/or subsets thereof, may be determined by microarray assay or any convenient screening method that enables simultaneous screening of several genes listed in Tables 4, 5 and/or 6, e.g., ten different AD-correlated genes, to several thousand AD-correlated genes, e.g., all of the genes listed in Table 5 or 6. For example, a diagnostic assay for AD or IAD may include screening for either up-regulation or down-regulation (as appropriate) of a subset of the genes listed in Table 5, such as for example, the genes listed in Table 6, or a smaller subset of the listed genes. Detection of a change in expression of a statistically significant percentage of genes shown herein to be correlated with IAD or AD is indicative that the patient has IAD or AD.

A subset of ADG or IADGs specifically linked to a process or system identified in Table 2, 3 or both (e.g, regulation of transcription, cell proliferation, oncogenesis, etc.), may be used in a microarray to test efficacy of a new compound targeted to slowing or reversing AD, either in experimental tests to develop new compounds, or as diagnostic or therapeutic guides. Similarly, such a subset of genes may be used in an assay, e.g., microarray-based assay, as a diagnostic tool for IAD or AD.

In a preferred embodiment an assay for changes in the pattern of expression of genes shown to be correlated with AD or IAD, such as a microarray-based assay, includes probing for expression of genes categorized as transcription factors (TFs). For example, a screen for AD or IAD may include probes for those genes listed in Table 4, or a subset of the genes in Table 4, such as the genes demarcated with an asterisk, the genes in boldface, the underlined genes, or combinations thereof.

The assay for probing expression of genes correlated with AD and/or IAD can be an RNA-based microarray for example. Changes in the pattern of gene expression of many genes can be identified simultaneously by hybridizing a control RNA sample and a sample of RNA obtained from a sample from a patient, such as for example a neural tissue sample, or brain biopsy to high density arrays containing several (e.g., five to ten or more), hundreds or thousands of oligonucleotide probes correlating to the genes or subsets of the genes identified herein as genes correlating to AD or IAD (Tables 5 and 6) (Cronin et al., (1996) Human Mutation 7:244-255; Kozal et al., (1996) Nature Medicine 2:753-759). The term “oligonucleotide” refers to a nucleic acid sequence of at least about 6 or 12 nucleotides to about 60 nucleotides, preferably about 15 to 30 nucleotides, and more preferably about 20 to 25 nucleotides, which can be used in PCR amplification or a hybridization assay, or a microarray. As used herein, oligonucleotide is substantially equivalent to the term “probe”, as commonly defined in the art. An oligonucleotide can be a cDNA sequence.

Hybridization conditions for detecting alterations in the expression pattern of ADGs or IADGs are readily determined by those of skill in the art. In general, high stringency hybridization conditions are used.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. below the melting temperature of the probe to about 20° C. to 25° C. below the melting temperature). One or more factors may be varied to generate conditions of either low or high stringency different from, but equivalent to, the above listed conditions.

Alternatively, the assay for detecting changes in expression patterns of ADGs and/or IADGs may be a protein- or an antibody-based assay in which the probes (antibodies) are specific for proteins encoded by the genes or a subset of the genes listed in Table 6. The test sample for an antibody-based assay, such as a microarray, Western blot analysis, ELISA screening, etc., would comprise screening proteins encoded by genes correlated with IAD or AD which are obtained from a body sample, such as spinal fluid, neural tissue or brain tissue, for an alteration in the amount of a plurality of proteins relative to the amount of the proteins in a control sample obtained from a non-AD individual or a pooled non-Ad sample. The sequences of the genes, and hence, the proteins encoded by the genes listed in Tables 4, 5 and 6 are known and publicly available, and are incorporated herein. Also, the person of ordinary skill in the art can use knowledge of the published sequences to generate appropriate probes for screening those genes of interest or may purchase commercially available probes for the genes of interest.

An alteration in the expression pattern of a statistically significant number of genes is indicative of a diagnosis of AD or IAD. Confirmation of a diagnosis of AD or IAD on the basis of an overall change in the pattern of gene expression of the tested genes correlated with AD or IAD may be made by MMSE for example, or other neurological test or test used to ascertain cognitive ability.

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The following Examples are presented in order to more fully illustrate the preferred embodiments of the invention. These examples should in no way be construed as limiting the scope of the invention, as defined by the appended claims.

### EXAMPLE 1

Human Brain Samples and Pathologic/Cognitive Assessment.

Hippocampal specimens used in this study were obtained at autopsy from 35 subjects (16 female and 19 male; Table I) through the Brain Bank of the Alzheimer's Disease Research Center at the University of Kentucky. At autopsy, coronal sections of the left hippocampus (3-5 mm) were immediately frozen in liquid nitrogen and stored at −80° C. until analyzed. Adjacent sections were fixed in 10% formalin and used for neuropathologic evaluation. Except for borderline AD subjects (see below), all AD patients met Alzheimer's Disease and Related Disorders Association criteria for the clinical diagnosis of AD and Consortium to Establish a Registry for Alzheimer's Disease and National Institute of Aging-Reagan Institute neuropathology criteria for the diagnosis of AD. The frozen hippocampal tissues were warmed to −20° C. to enable dissection of CA1 and CA3 under a Zeiss surgical microscope.

TABLE 1

| | Control | Incipient | Moderate | Severe |
|---|---|---|---|---|
| | (n = 9) | (n = 7) | (n = 8) | (n = 7+ |
| Age | 85.3 ± 2.7 | 90 ± 2.1 | 83.4 ± 1.1 | 84 ± 4.0 |
| NFT | 2.7 ± 1.0 | 9.4 ± 1.8 | 25.6 ± 3.5 | 32.7 ± 7.2 |
| Braak | 2.1 ± 0.4 | 5 ± 0.4 | 5.6 ± 0.2 | 5.9 ± 0.1 |
| NMSE | 27.7 ± 0.5 | 24.3 ± 1.1 | 16.5 ± 0.6 | 6 ± 1.4 |
| PMI | 2.6 ± 0.2 | 3.3 ± 0.6 | 3.2 ± 0.2 | 3 ± 0.1 |

Values are mean ± SEM.
PMI = postmortem interval.

The MiniMental State Examination (MMSE) is a reliable index of AD-related cognitive status at a given point in time (Clark et al. (1999) Arch. Neurol., 56:857-862). However, its rate of decline varies with severity, and mildly impaired patients show little MMSE decline even after several years (Clark et al., supra). Recent MMSE data were available for most subjects but, in subjects for whom the interval between the most recent MMSE score and death was >1 year, the MMSE score was adjusted downward by one point per year. This approach likely underestimates MMSE decline for severely affected patients but seemed suitable for this study, given the slow MMSE decline in less impaired subjects (Clark et al., supra) and the focus on such subjects. Postmortem scores on AD-related pathologic indices for Braak staging, hippocampal neurofibrillary tangles (NFT), and diffuse and neuritic senile plaques were determined as described (Geddes et al. (1997) Neurobiol. Aging, 18:S99-S105). The MMSE and NFT values were selected as primary markers for quantifying AD progression because of the Braak scale's limited range and because the observed N F T results correlated more closely with the MMSE (r=0.45) than did the observed plaque values (r=0.19), consistent with prior findings (See Hyman, B. T. (1997) Neurobiol. Aging, 18:S27-S32). Further, the evidence that soluble rather than deposited Beta Amyloid may be more relevant to cognitive impairment is mounting (Klein et al. (2001) Trends Neurosci., 24:219-224; Price et al. (1998) Annu. Rev. Neurosci., 21:479-505; Morgan, D. (2003) Neurochem., 28:1029-1038).

Based primarily on MMSE criteria (Mitchell, et al., Ann. Neurol., 51:182-189; Clark et al., supra), subjects were categorized initially into four groups, termed “Control” (MMSE>25), “Incipient AD” (MMSE 20-26), “Moderate AD” (MMSE 14-19), and “Severe AD”(MMSE<14)(Table 1). Several borderline cases (e.g., MMSE=26) were assigned based on NFT, amyloid plaque, and Braak stage data. In addition, four subjects exhibited more cognitive deterioration (MMSE<20) than expected from their NFT or amyloid scores. Because these subjects were potentially affected by confounding conditions, they were excluded from the analyses, leaving η=31 overall.

RNA Isolation and Affymetrix GeneChip Processing.

Procedures for total RNA isolation, labeling, and microarray processing were similar to those described (Blalock et al., supra), except that human GeneChips (HG-U133A) and MICROARRAY SUITE 5 (MAS5; Affymetrix, (2001) Affymetrix Microarray Suite User's Guide (Affymetrix, Santa Clara, Calif., version 5) were used. Each subject's CA1 subfield RNA was processed and run on a separate chip. An average yield of 55 μg of biotin-labeled cRNA target was obtained from 8 μg of total RNA each per CA 1 sample, of which 20 μg of cRNA was applied to one array. cRNA yield did not differ significantly among groups (P=0.32), but the most severe AD group exhibited a trend toward lower cRNA levels, possibly reflecting greater cellular degeneration.

Microarray Data Analysis.

Scaling and noise analyses were performed as described (Blalock et al. supra) and Affymetrix algorithms for signal intensity and presence P values (Affymetrix, supra), respectively, were used to determine expression (relative abundance) and detection reliability of transcripts. A gene probe set was rated “present” if it was detected on at least four chips in the study. Individual values were blanked and treated as missing values if they were >2 SD away from the group mean. Finally, probe sets were considered “genes” if they had been assigned a “gene symbol” annotation (Affymetrix database). Pearson's correlation tests and ANOVAs were performed in EXCEL 9.0 data copied from the MASS pivot table, as described (Blalock et al., supra).

Biological Process Categorization by Gene Ontology.

As noted, microarray studies face substantial false-positive concerns because of the large multiple comparison error. Conversely, however, they can also strengthen statistical confidence by providing evidence of coregulation of multiple genes that re related by function of pathway (See Ashburner et al., (2000) Nat. Genet., 25:25-29). In the present study, a new software tool, the EXPRESSION ANALYSIS SYSTEMATIC EXPLORER (EASE)(available from NIAID), to assign identified genes to “GO: Biological Process” categories of the Gene Ontology Consortium (Ashburner et al., supra) and to test statistically (EASE Score, a modified Fisher's exact test) for significant coregulation (overrepresentation) of identified genes within each biological process category.

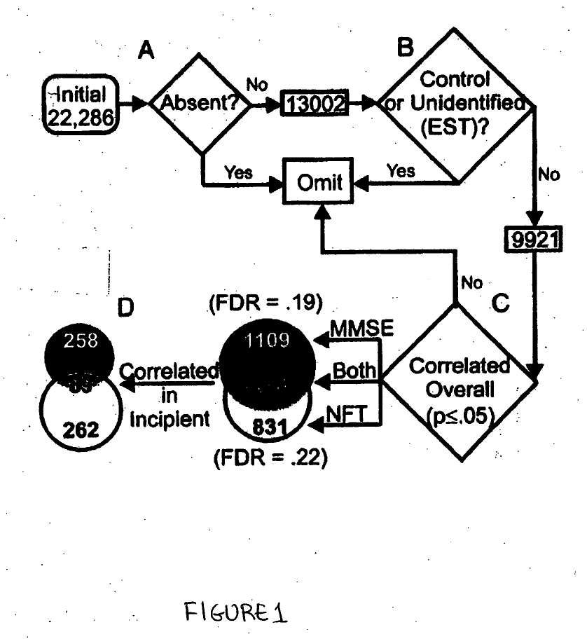

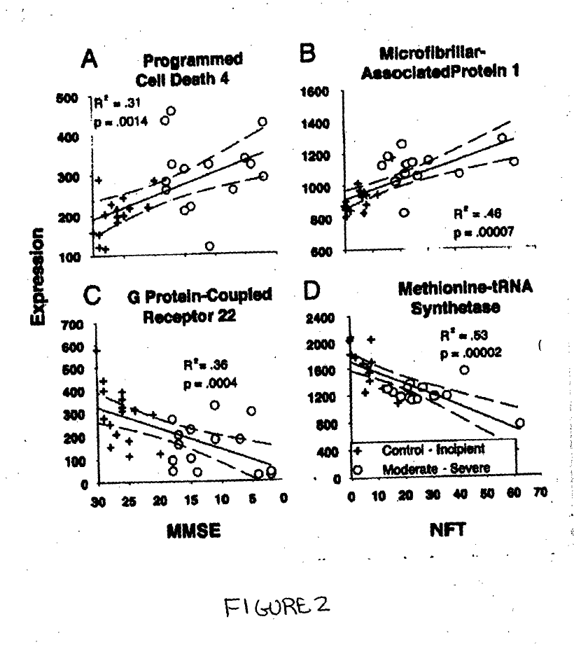

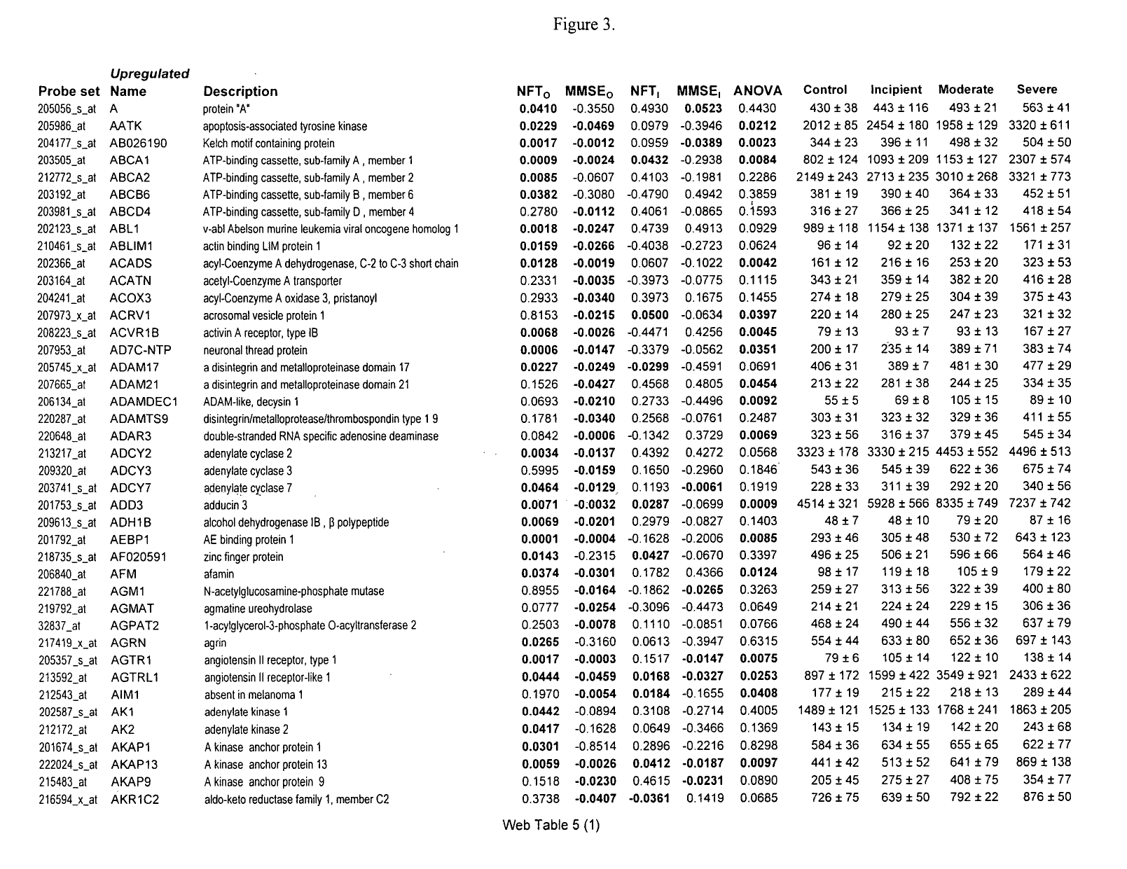

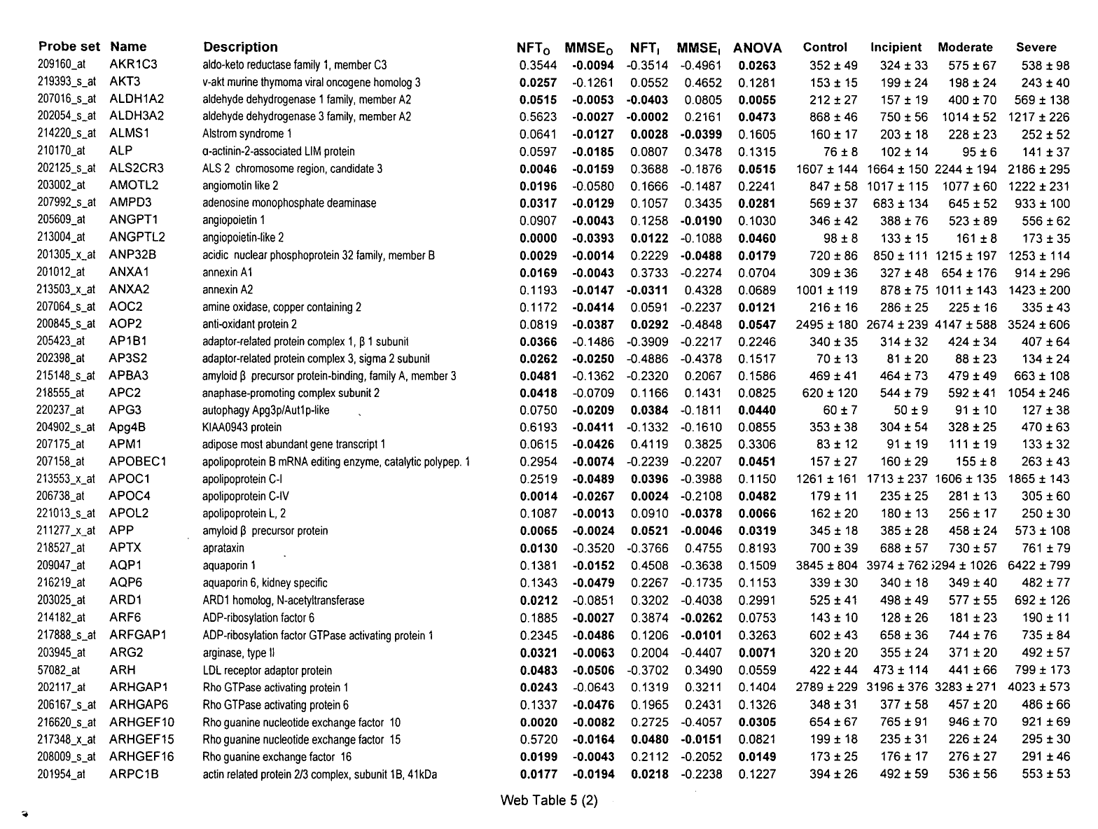

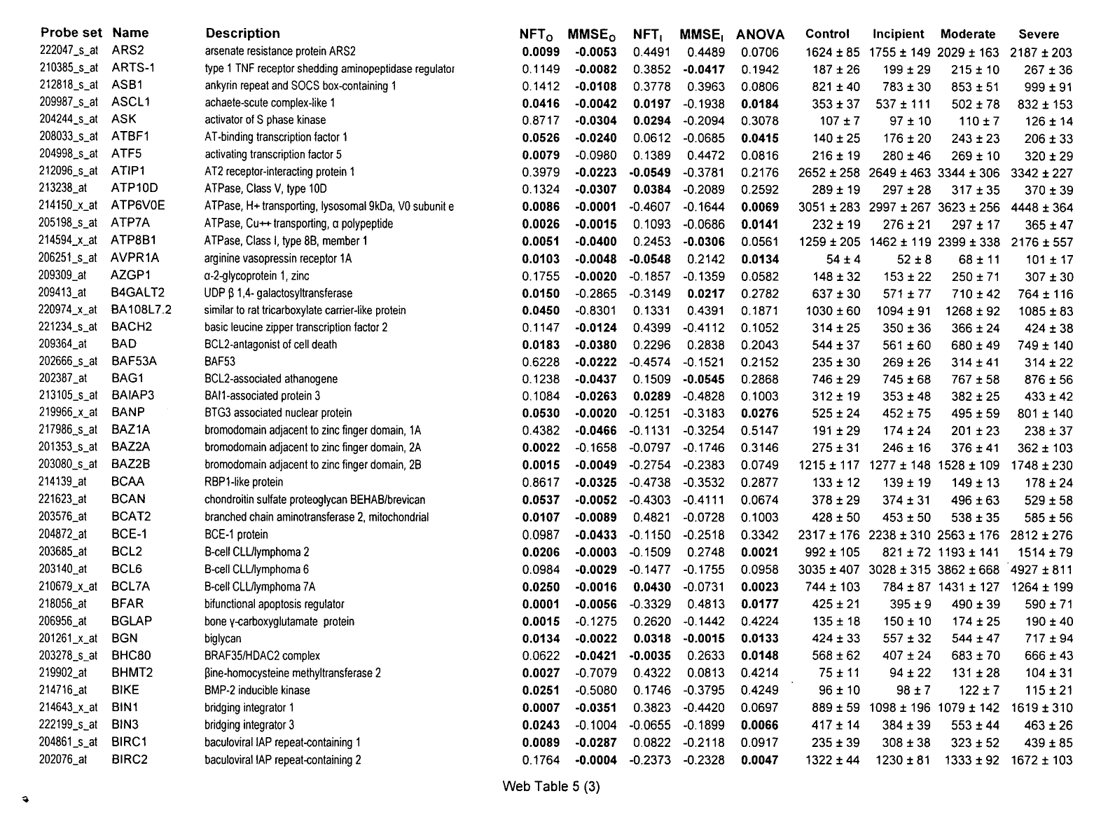

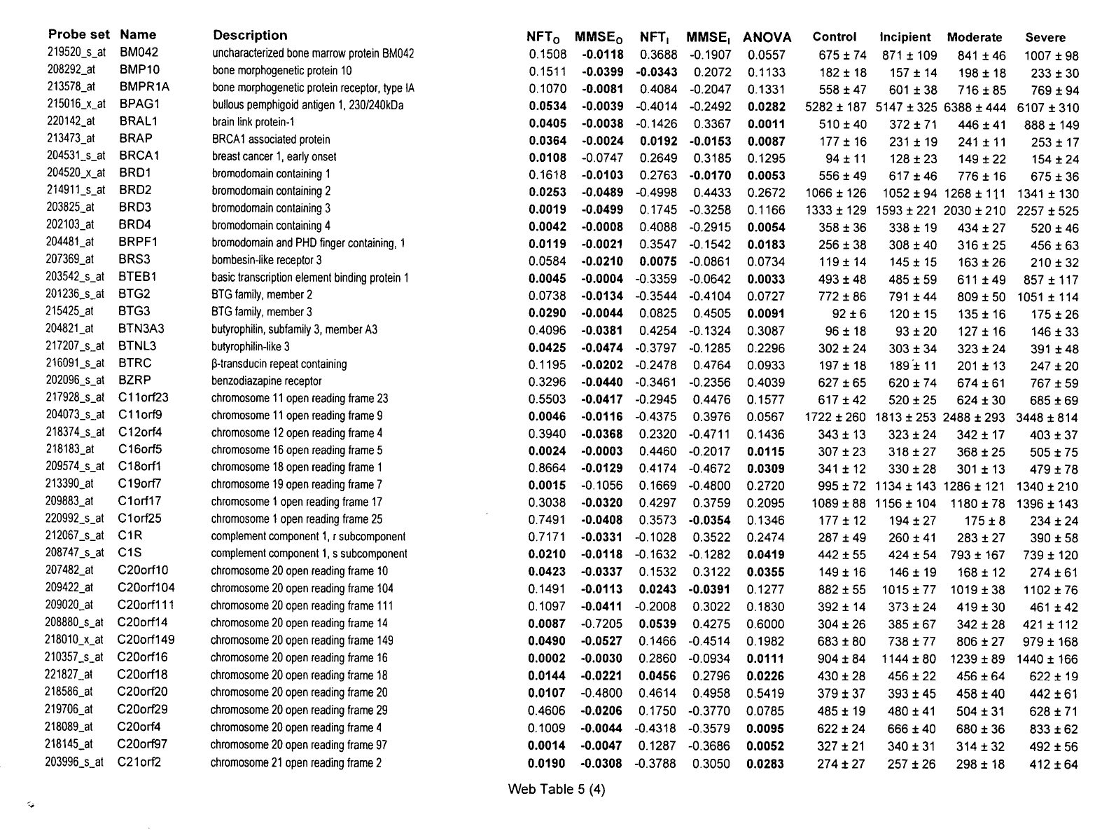

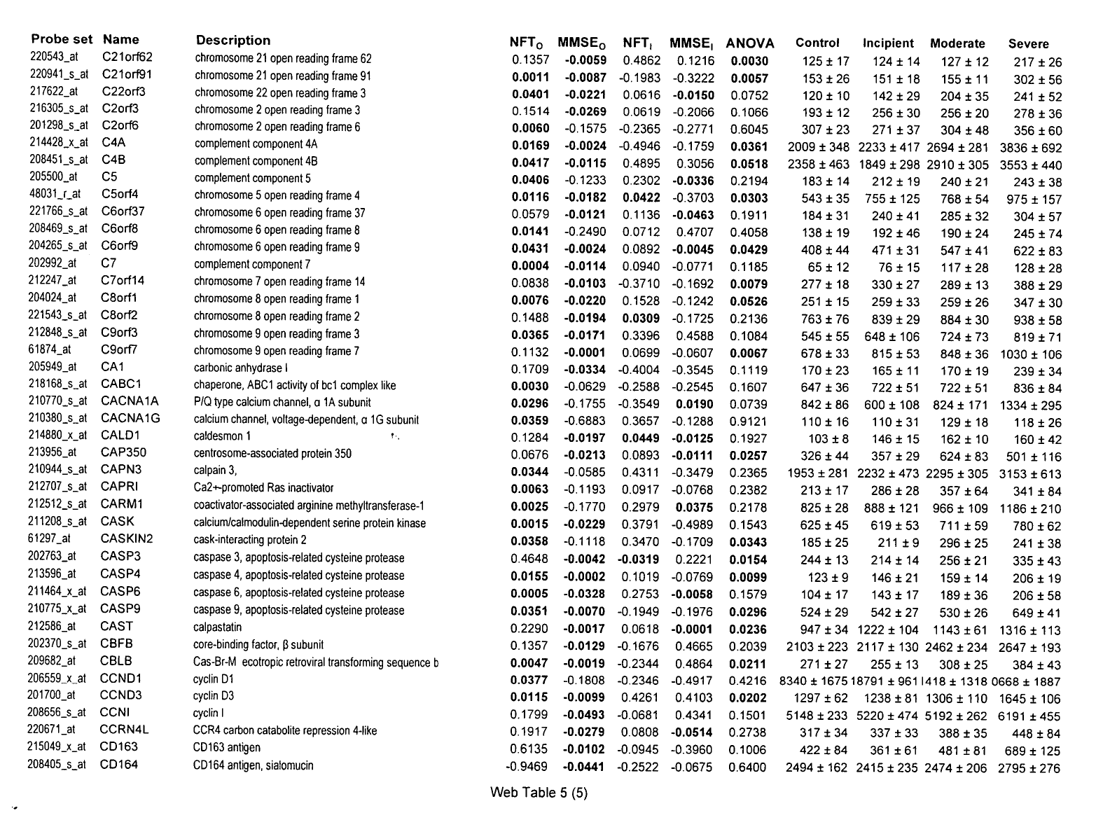

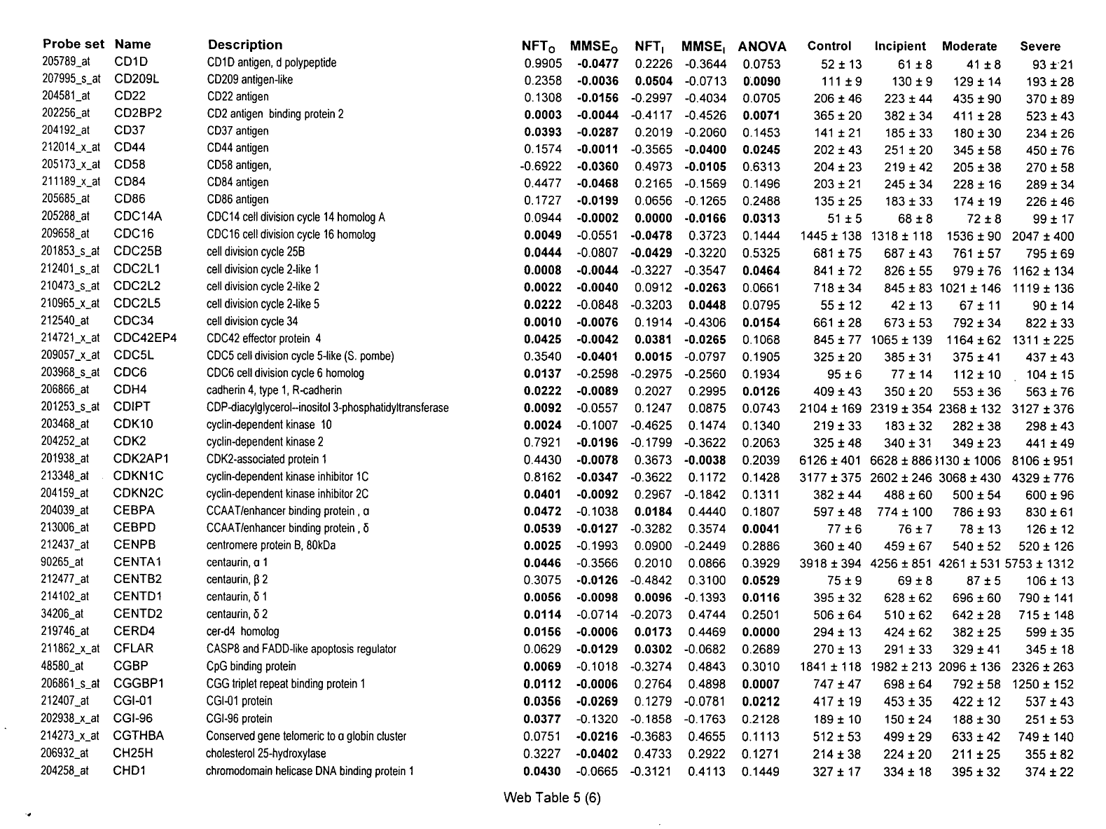

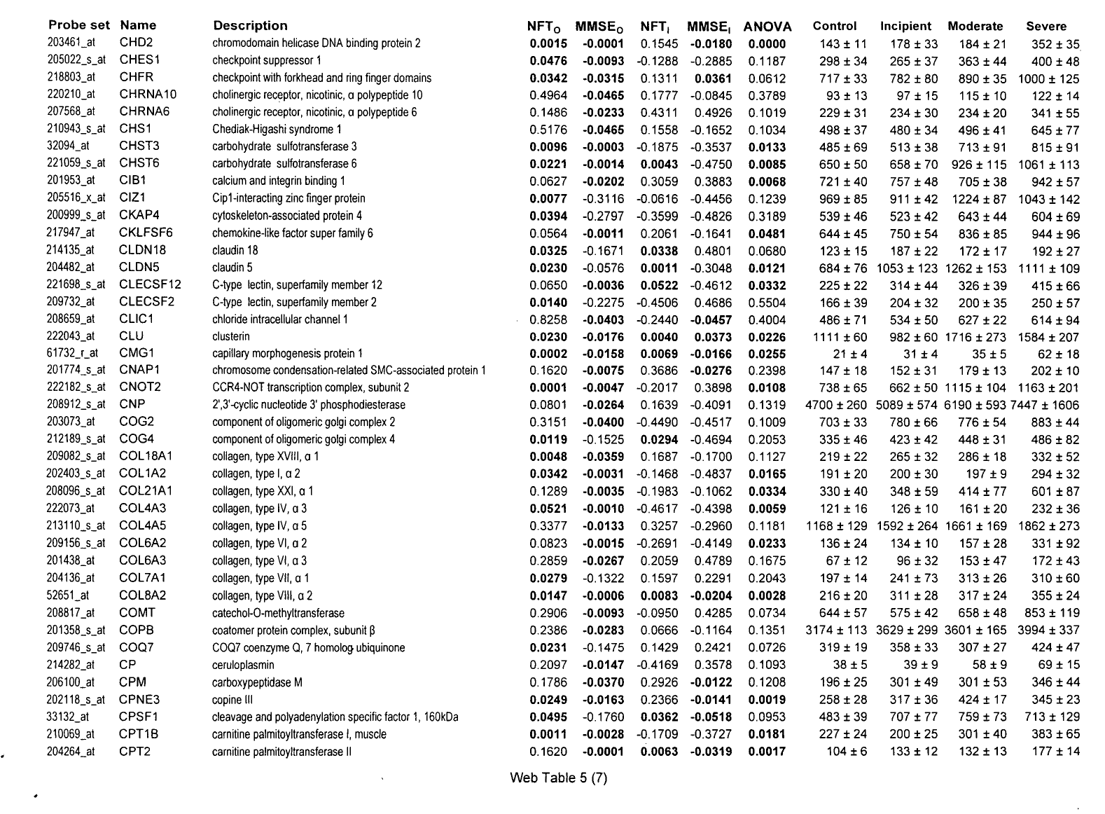

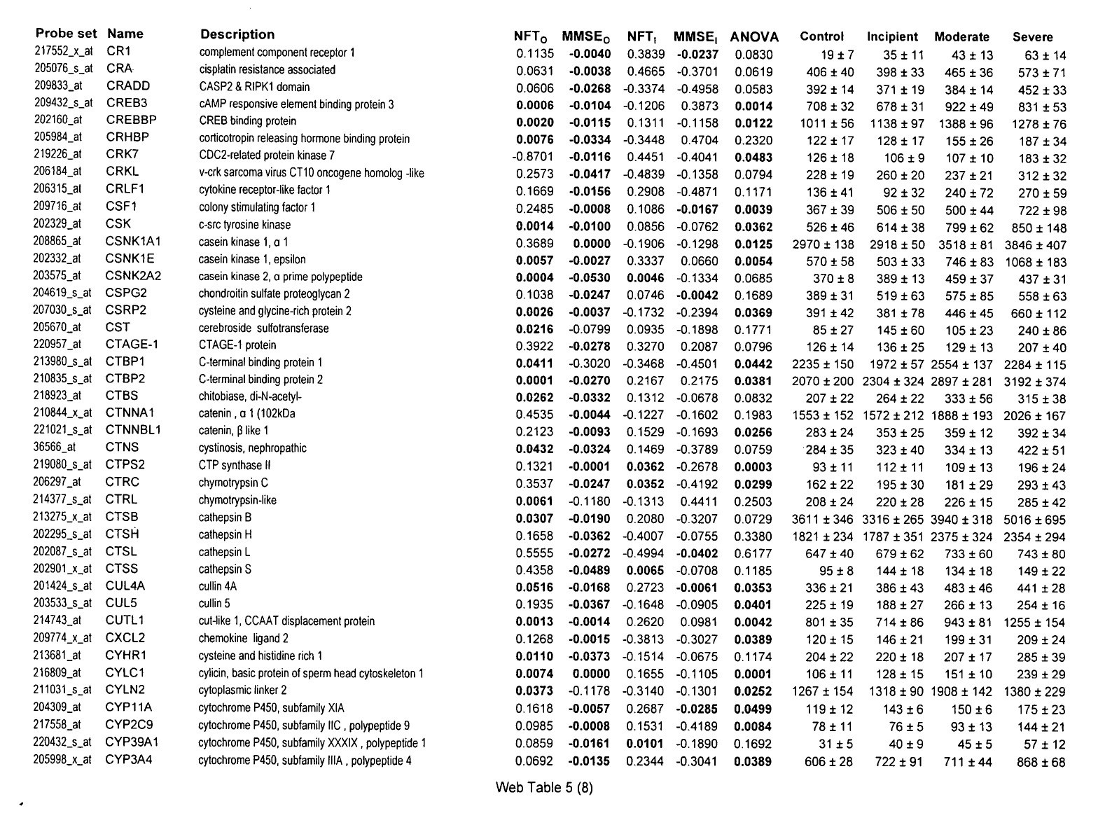

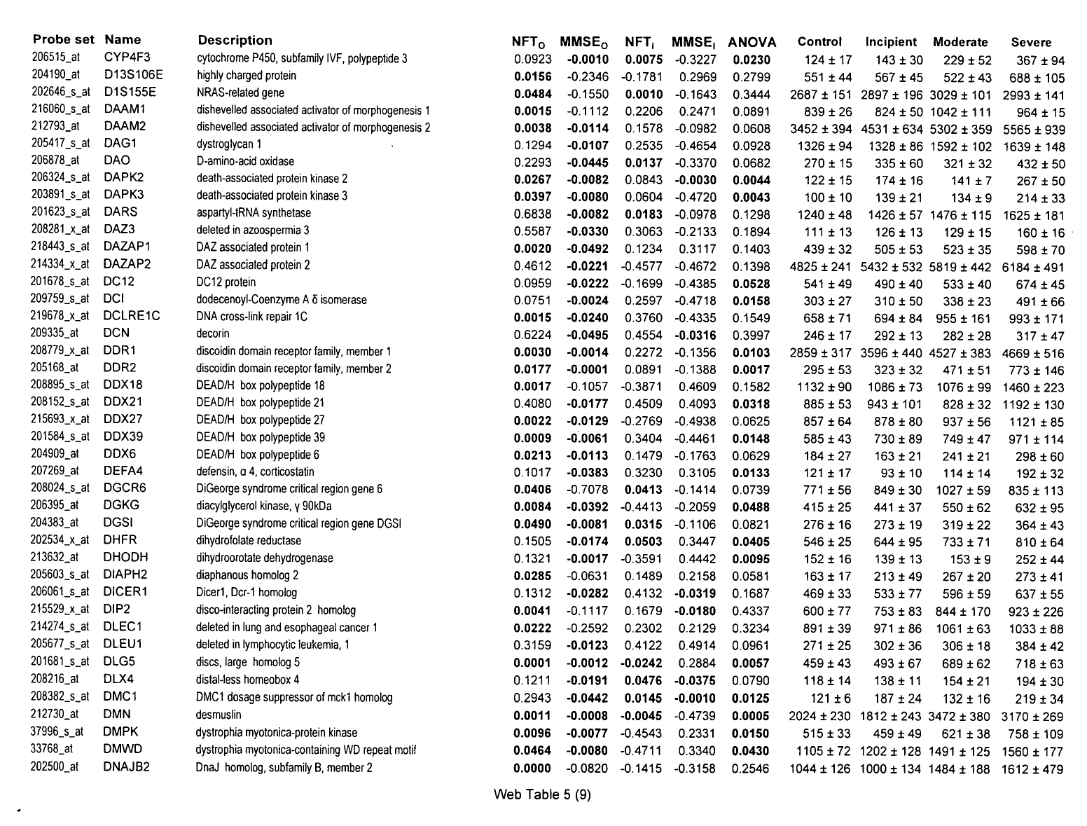

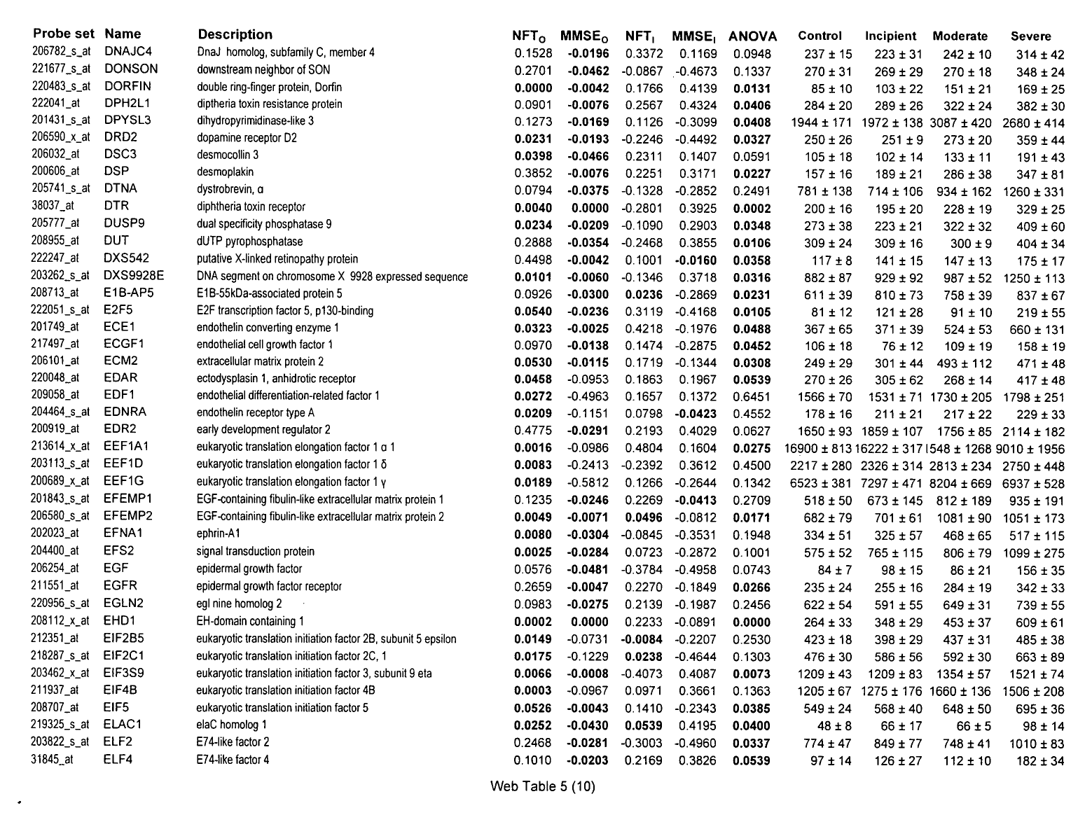

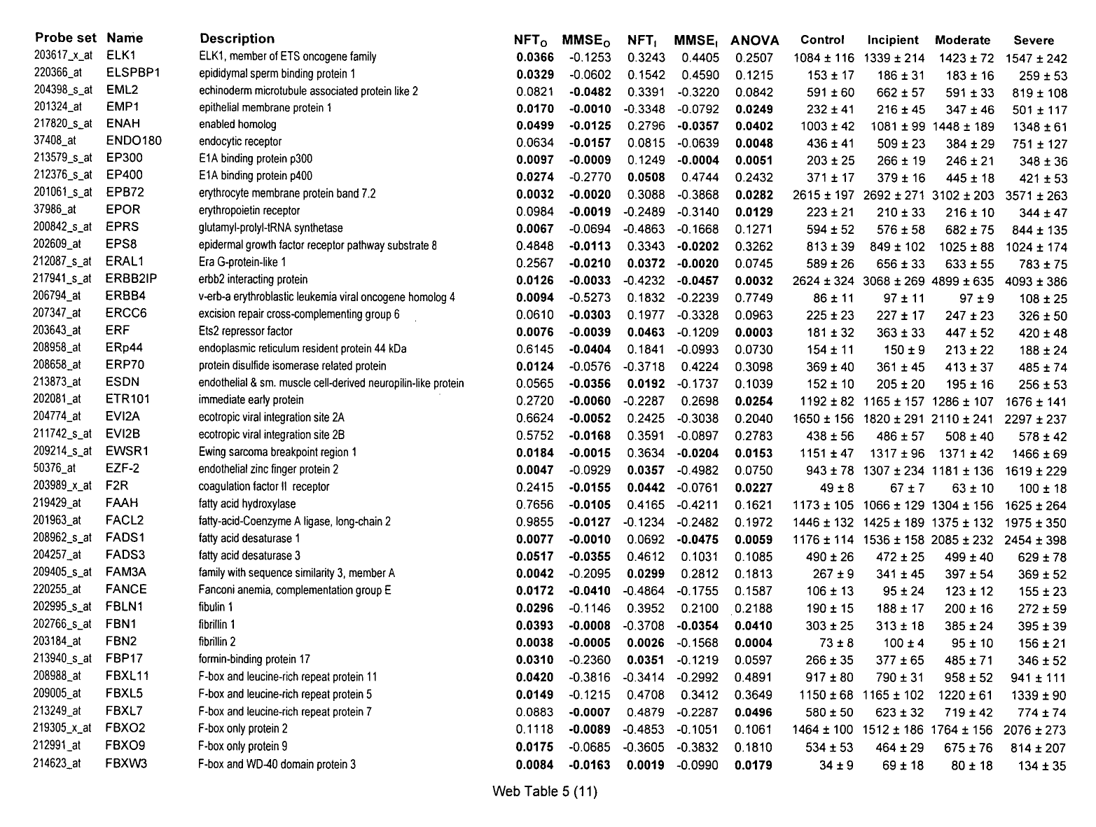

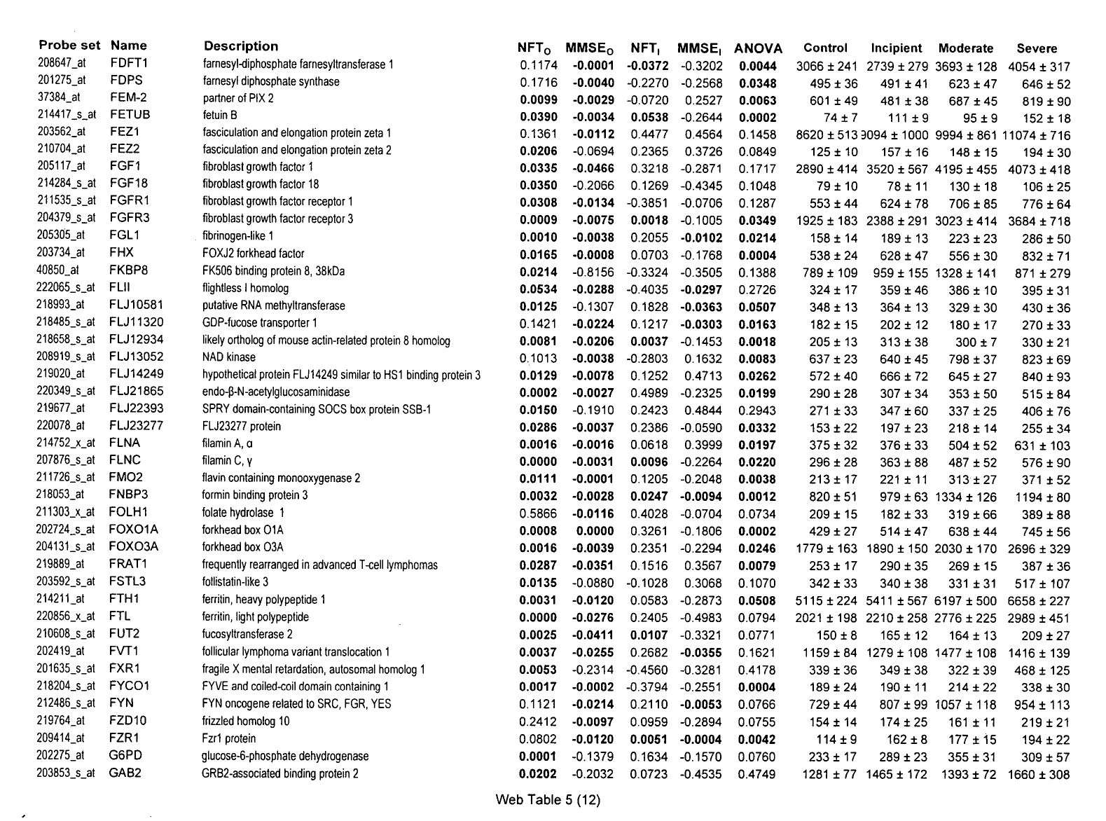

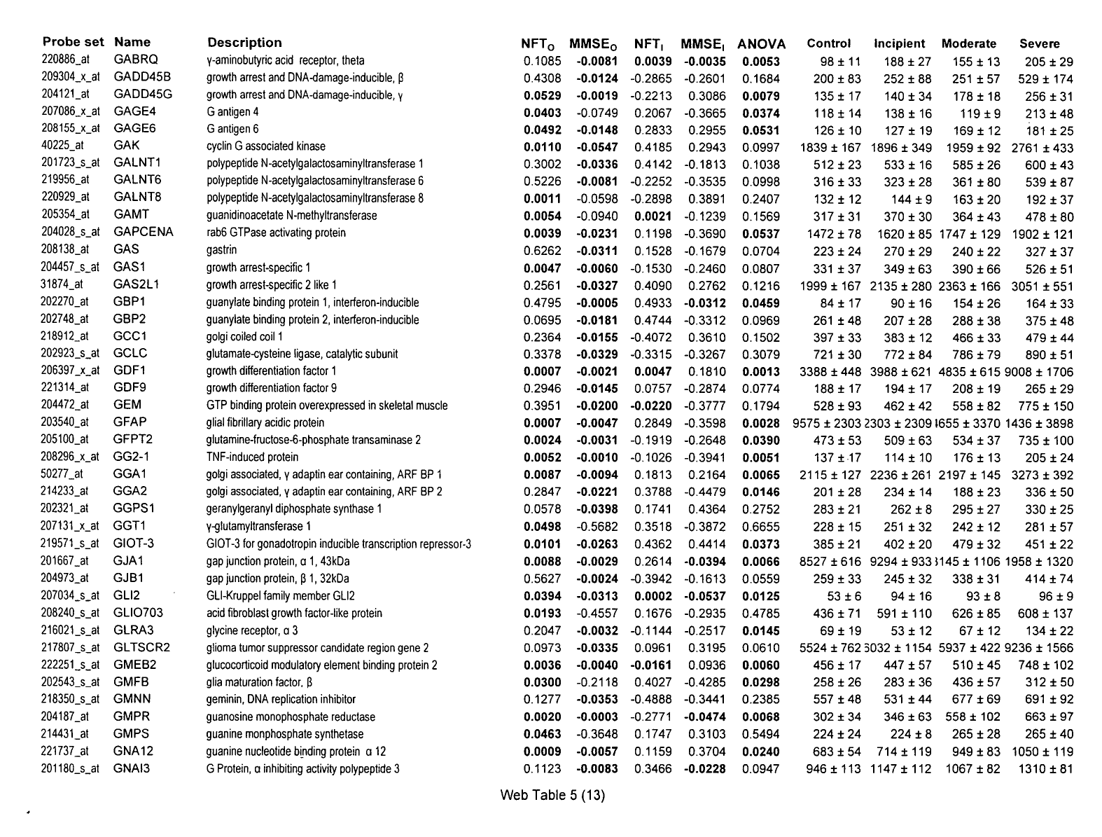

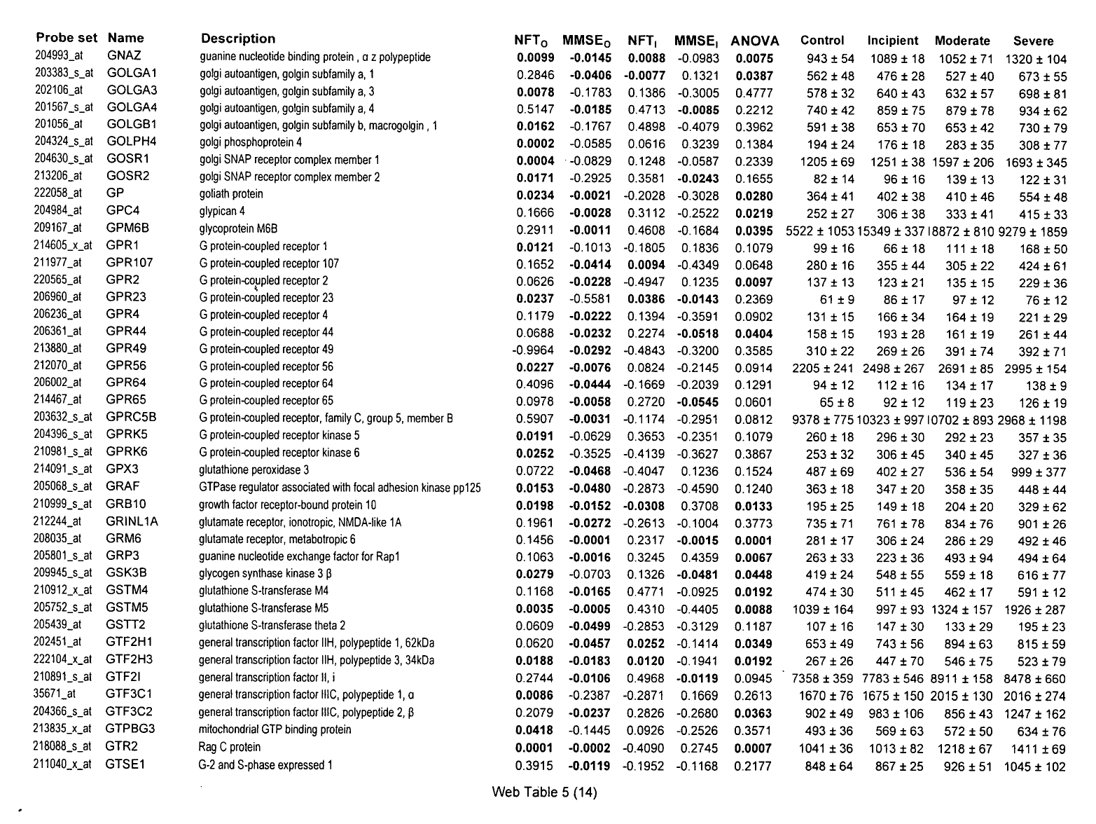

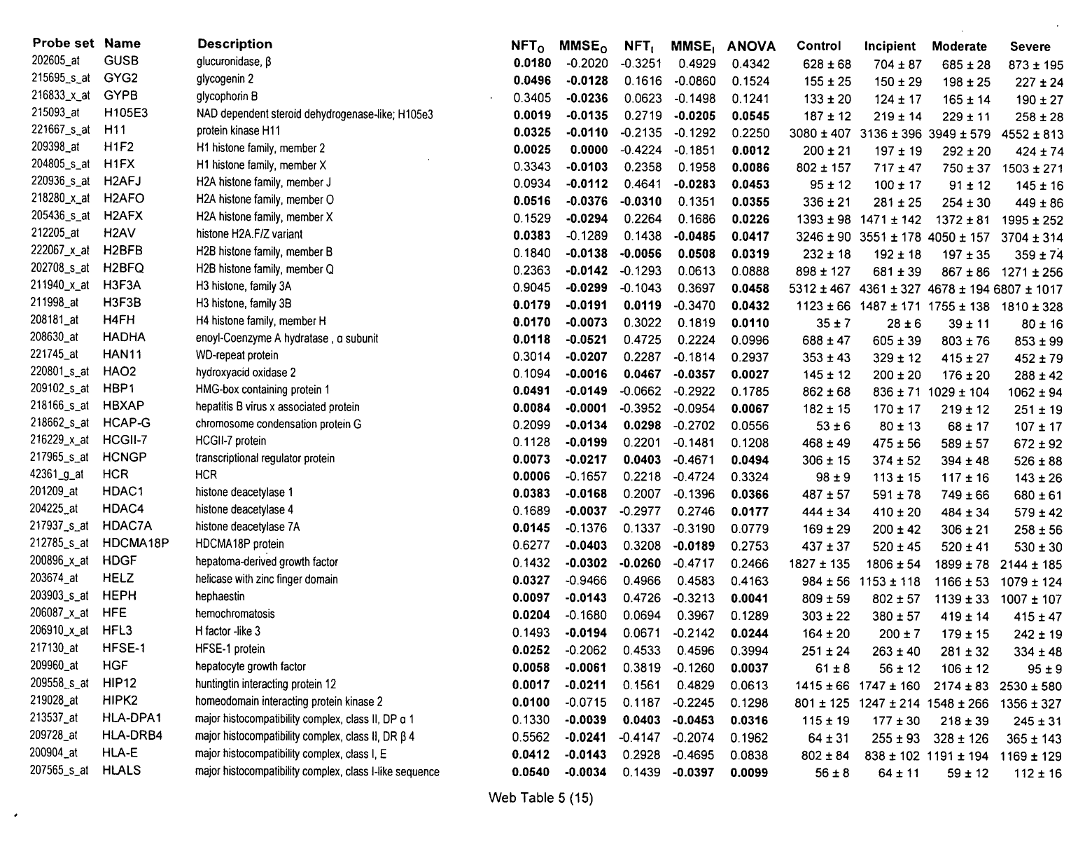

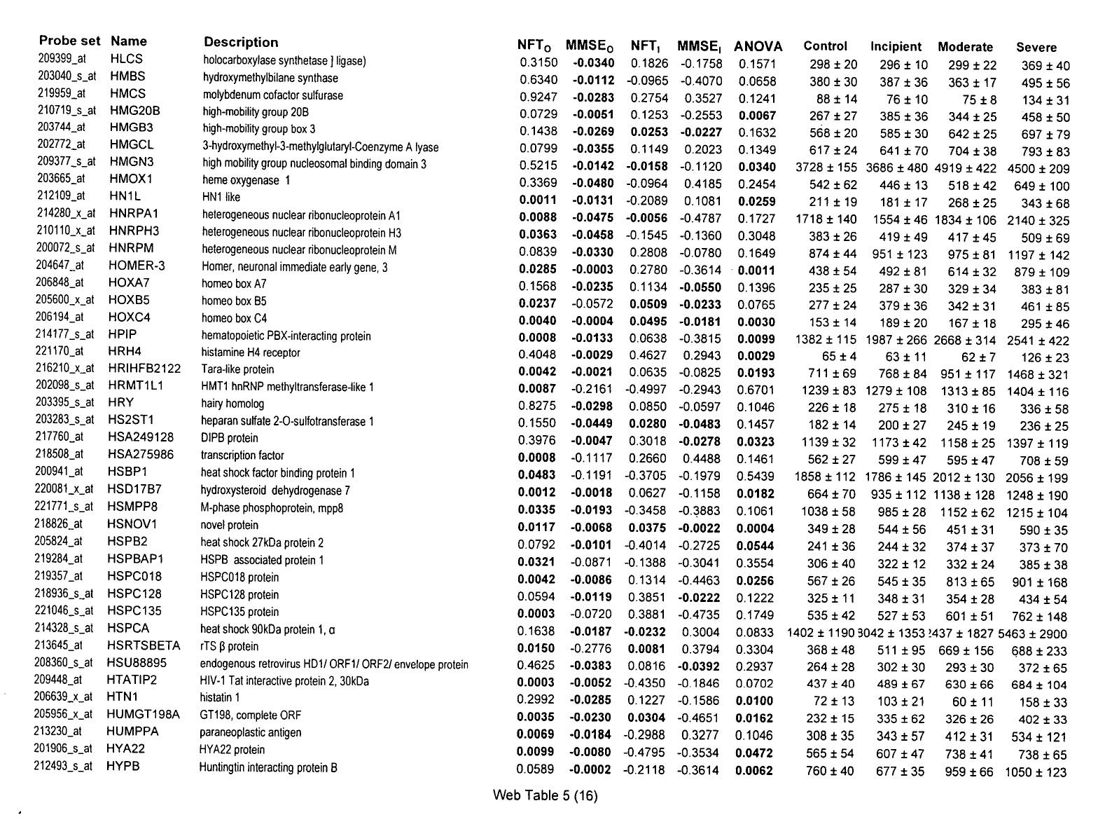

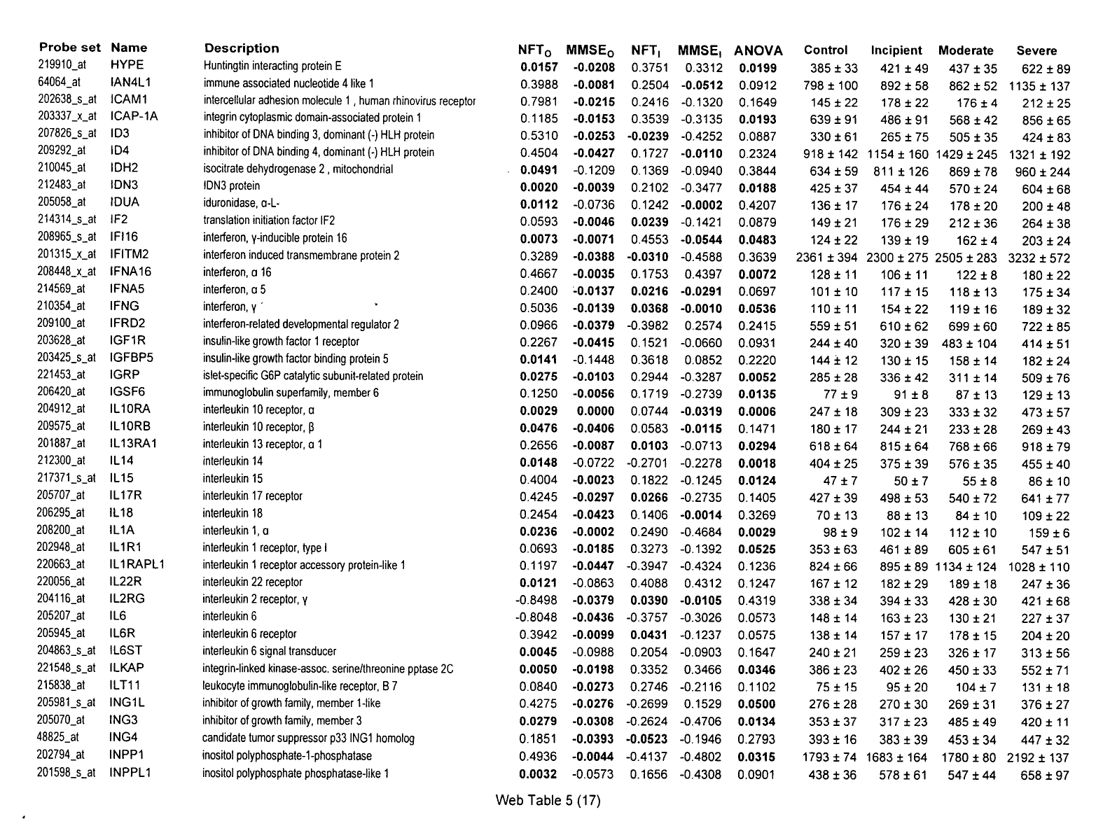

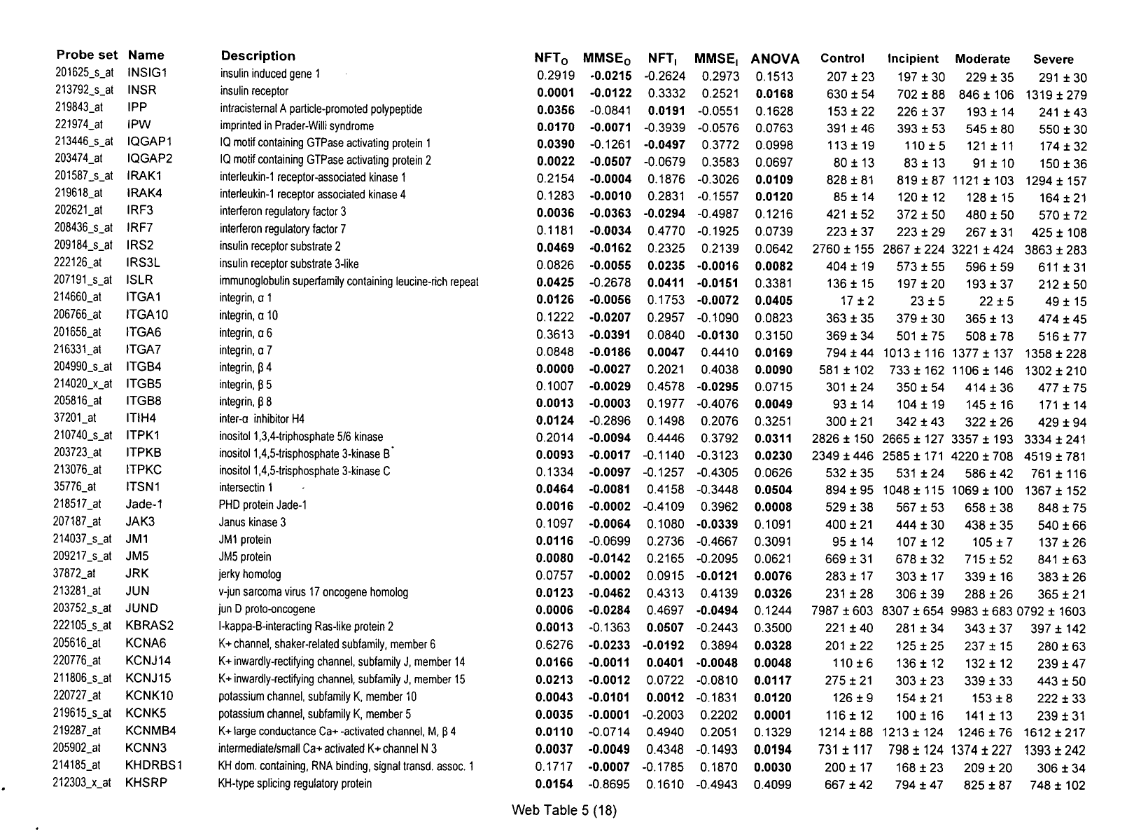

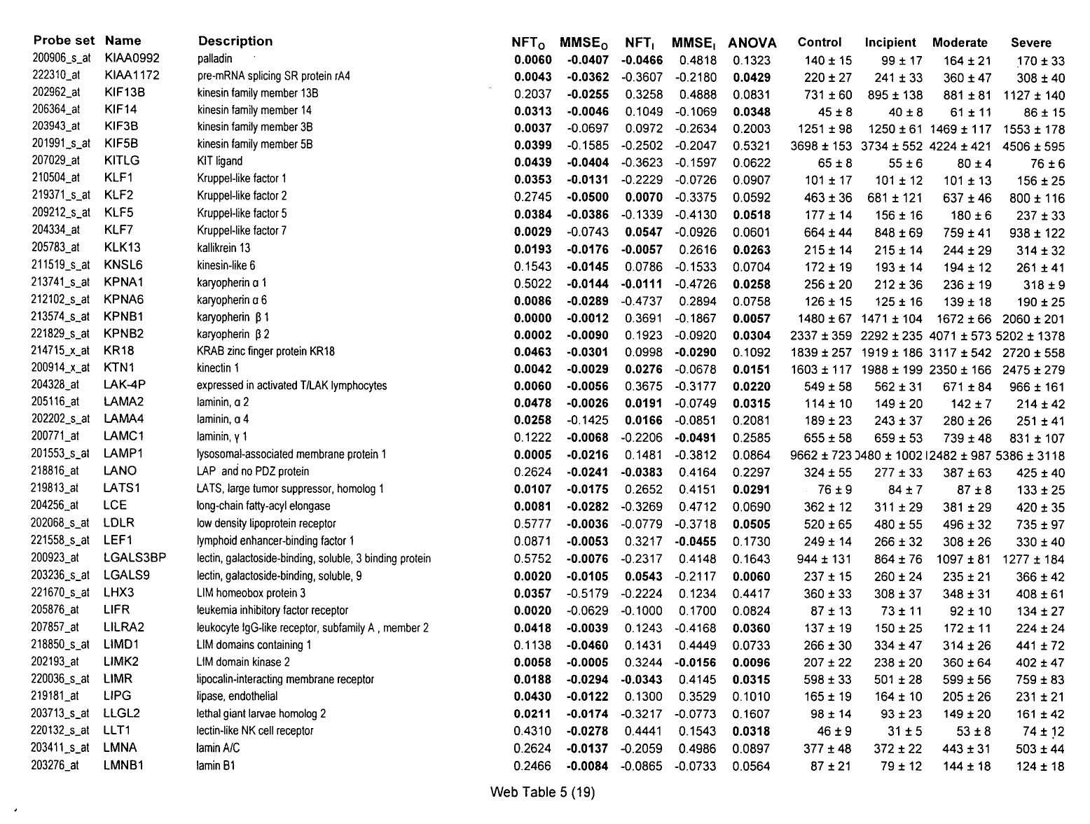

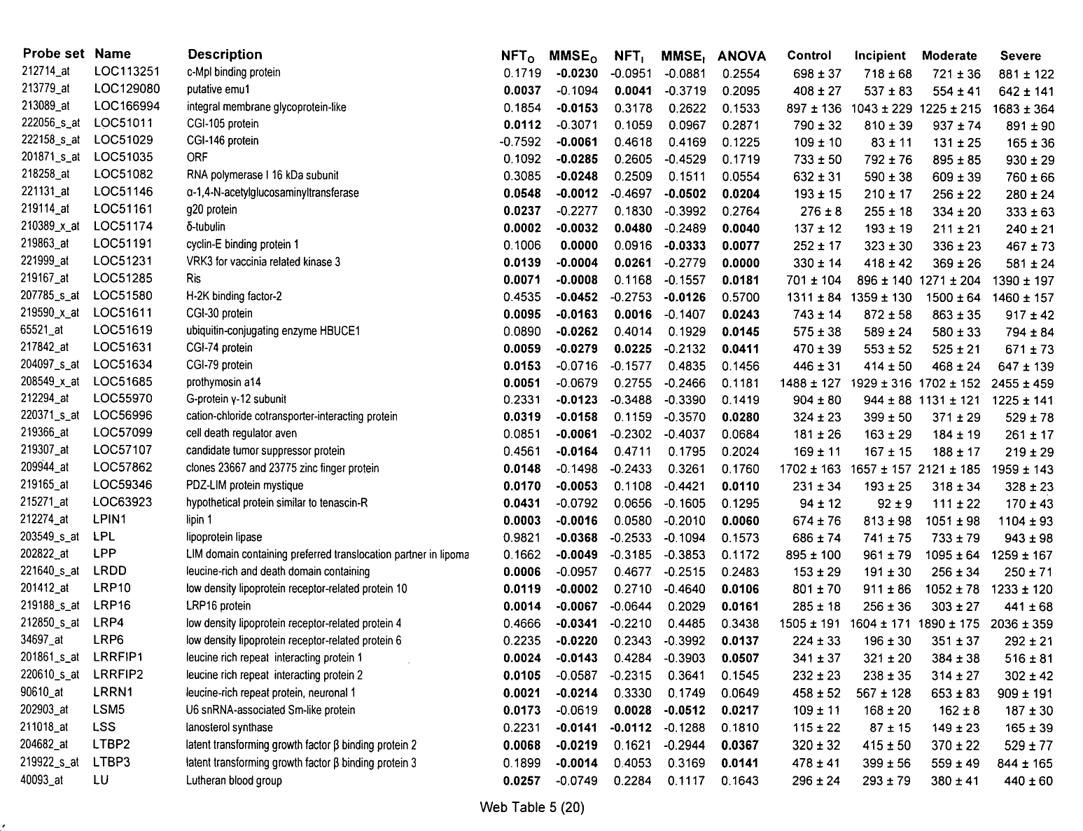

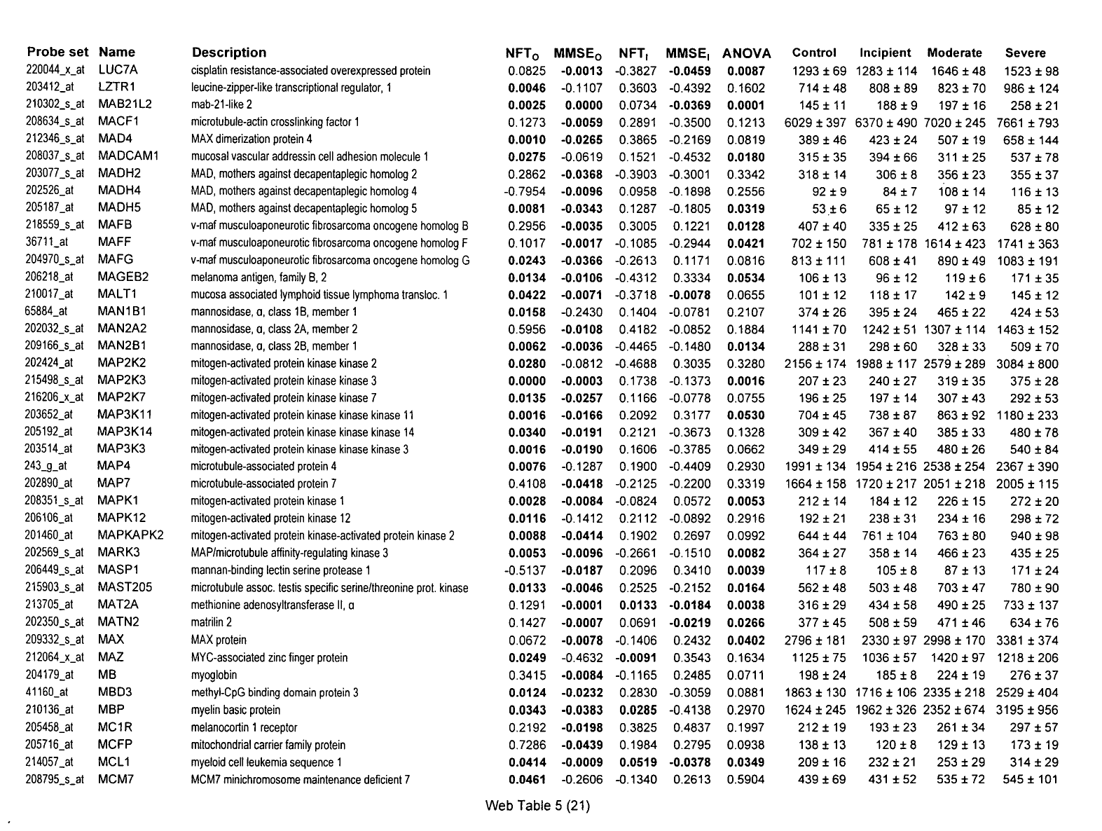

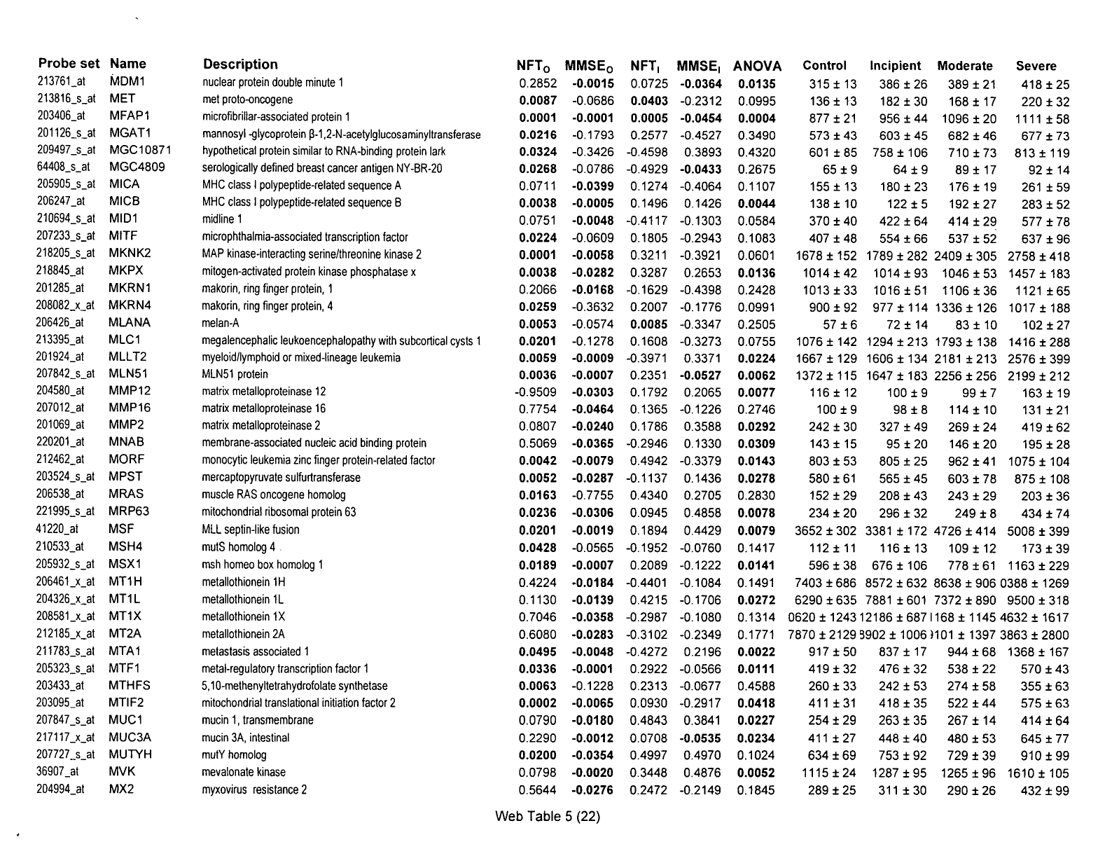

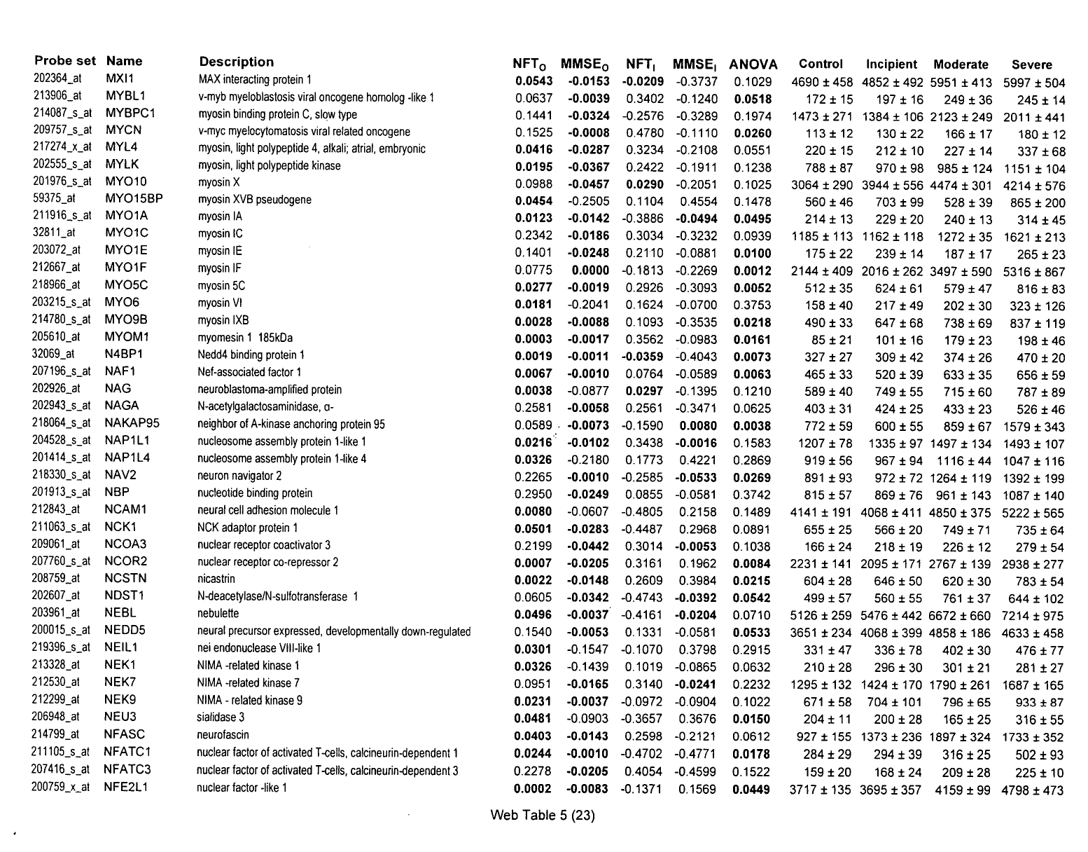

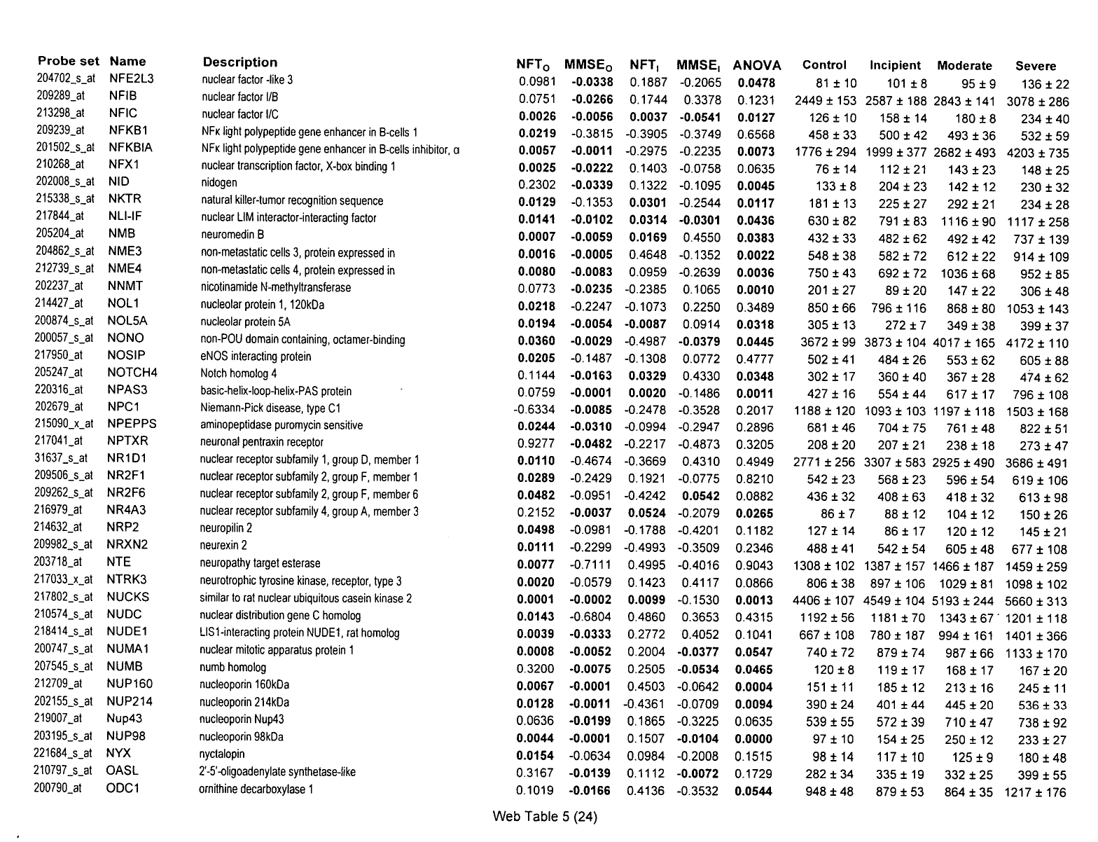

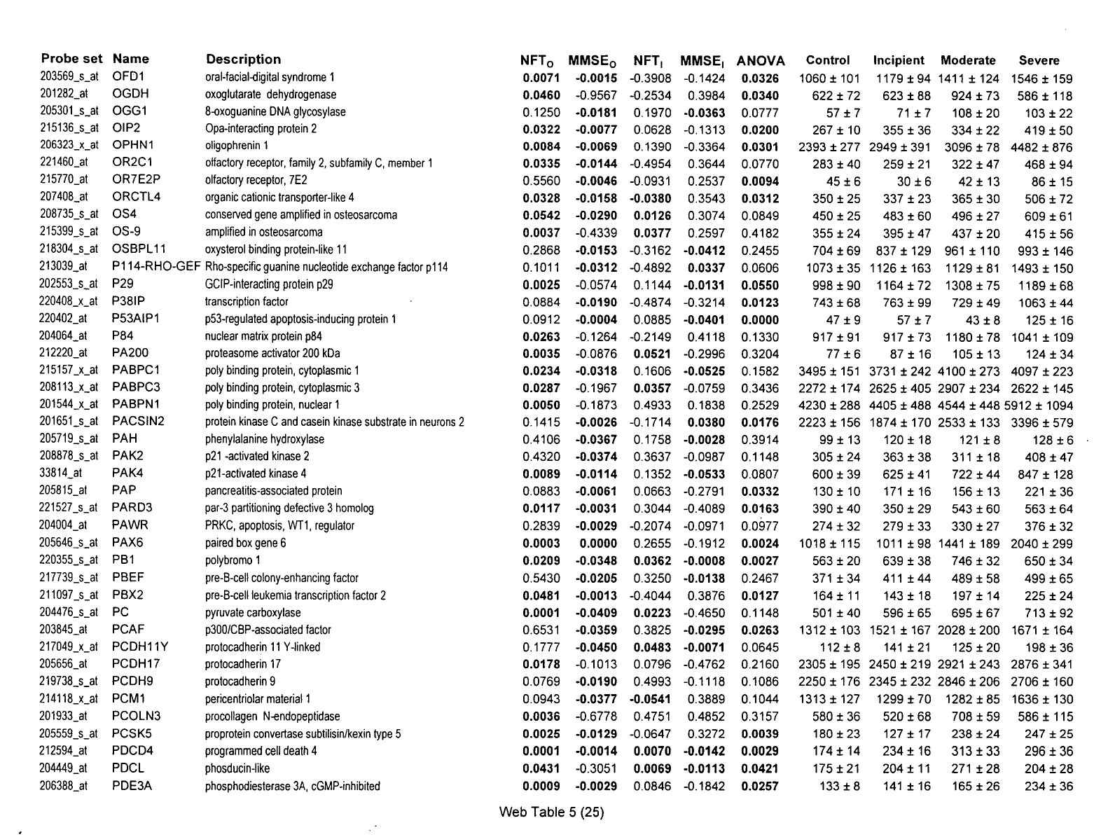

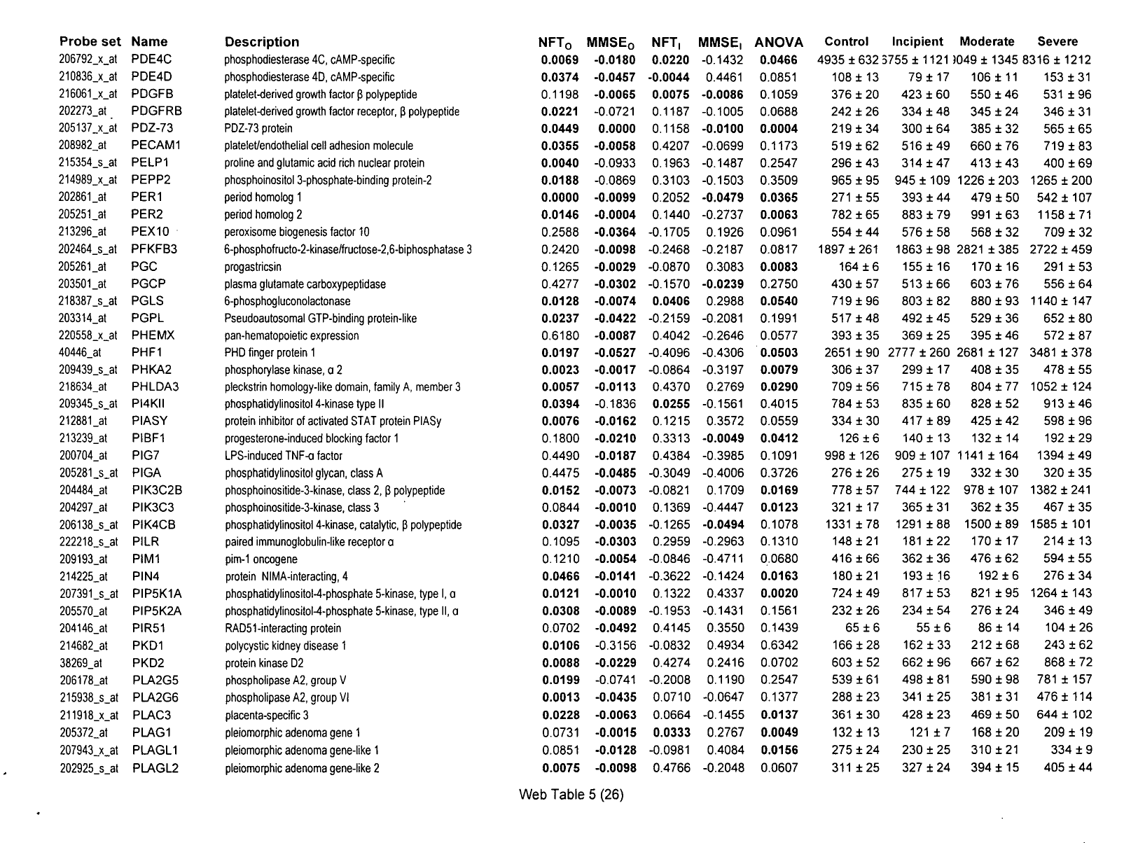

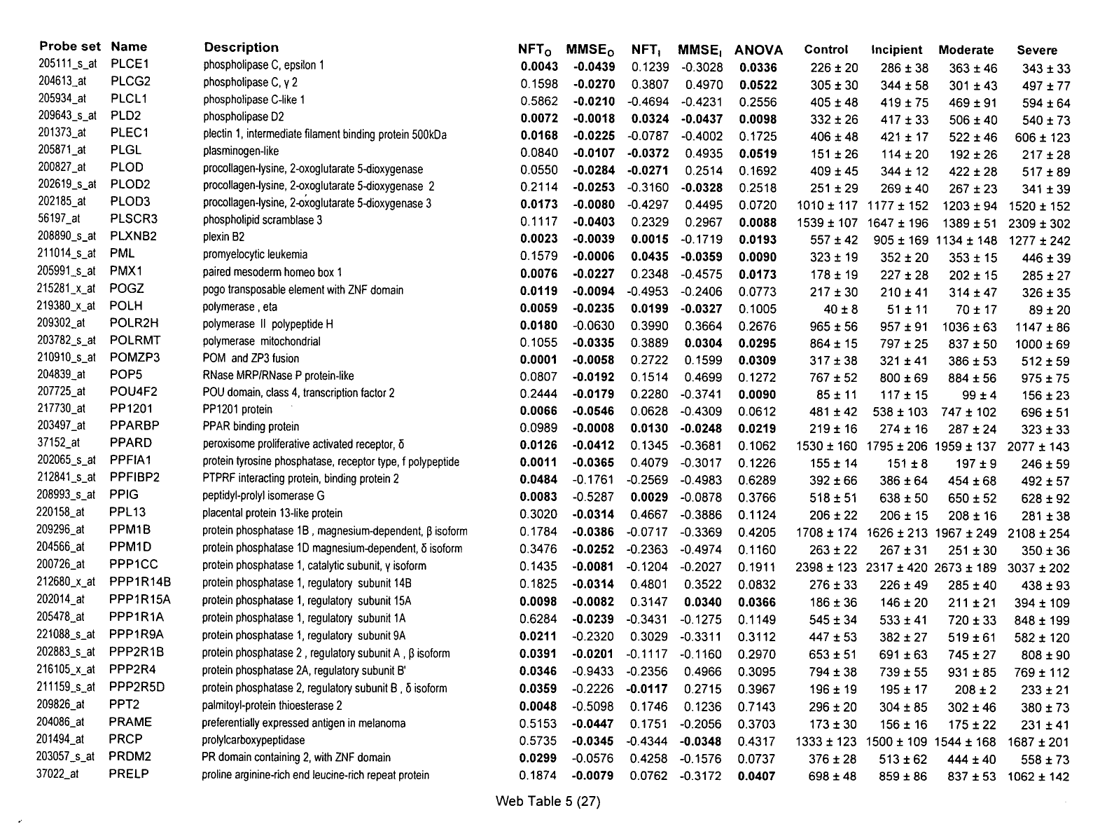

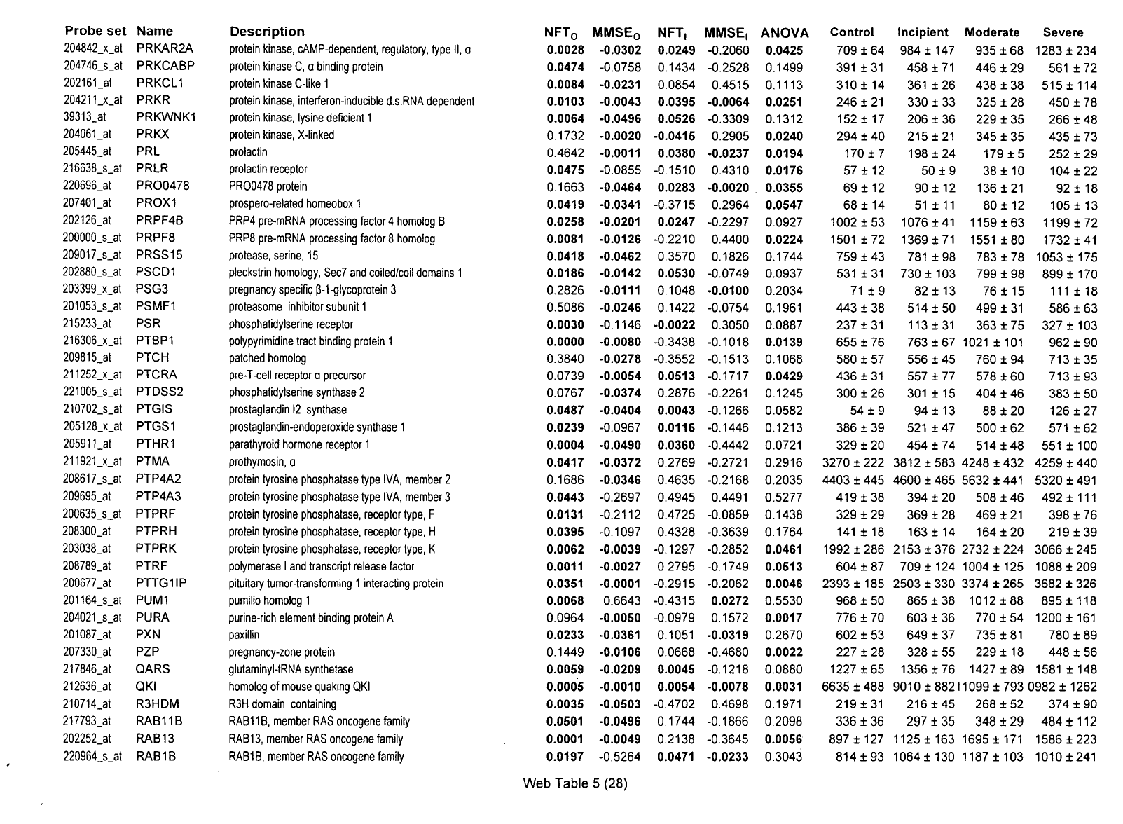

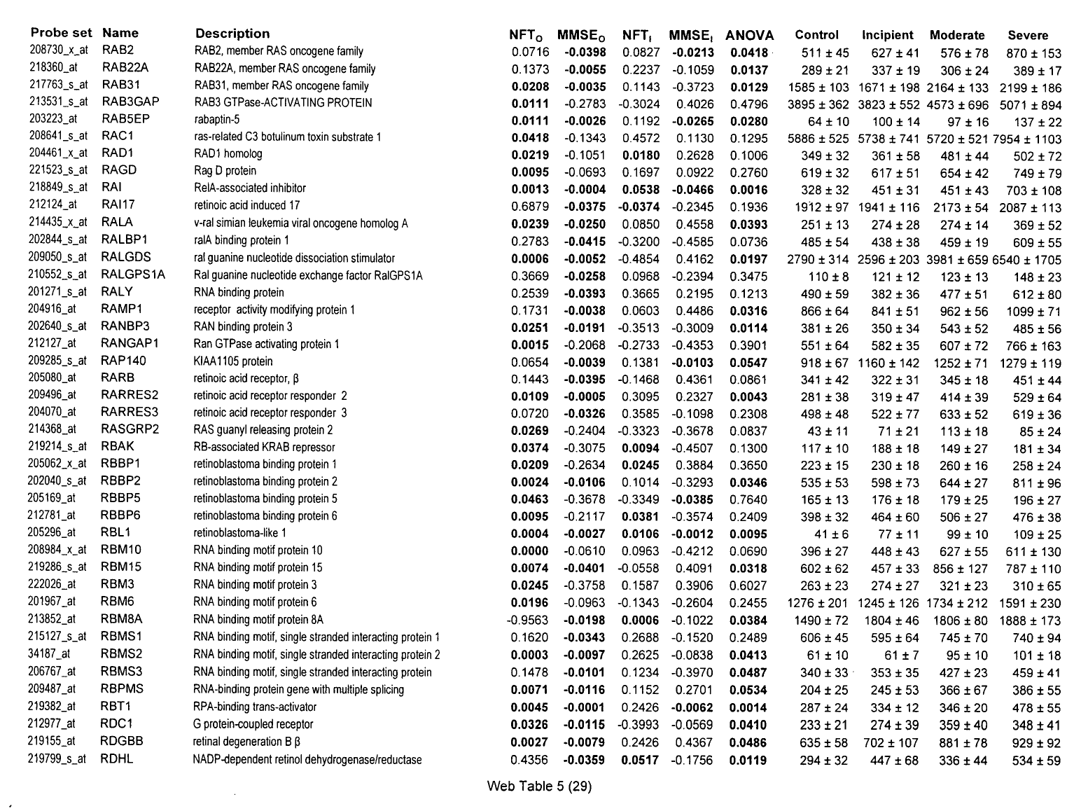

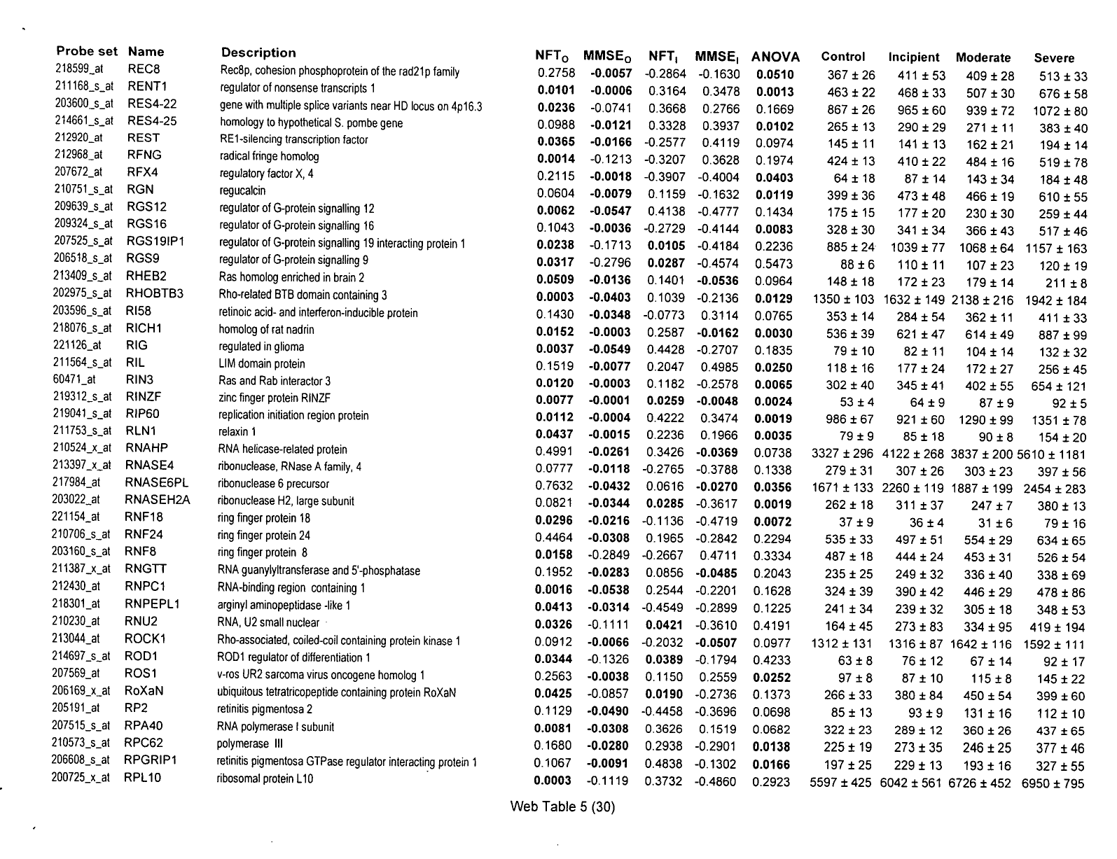

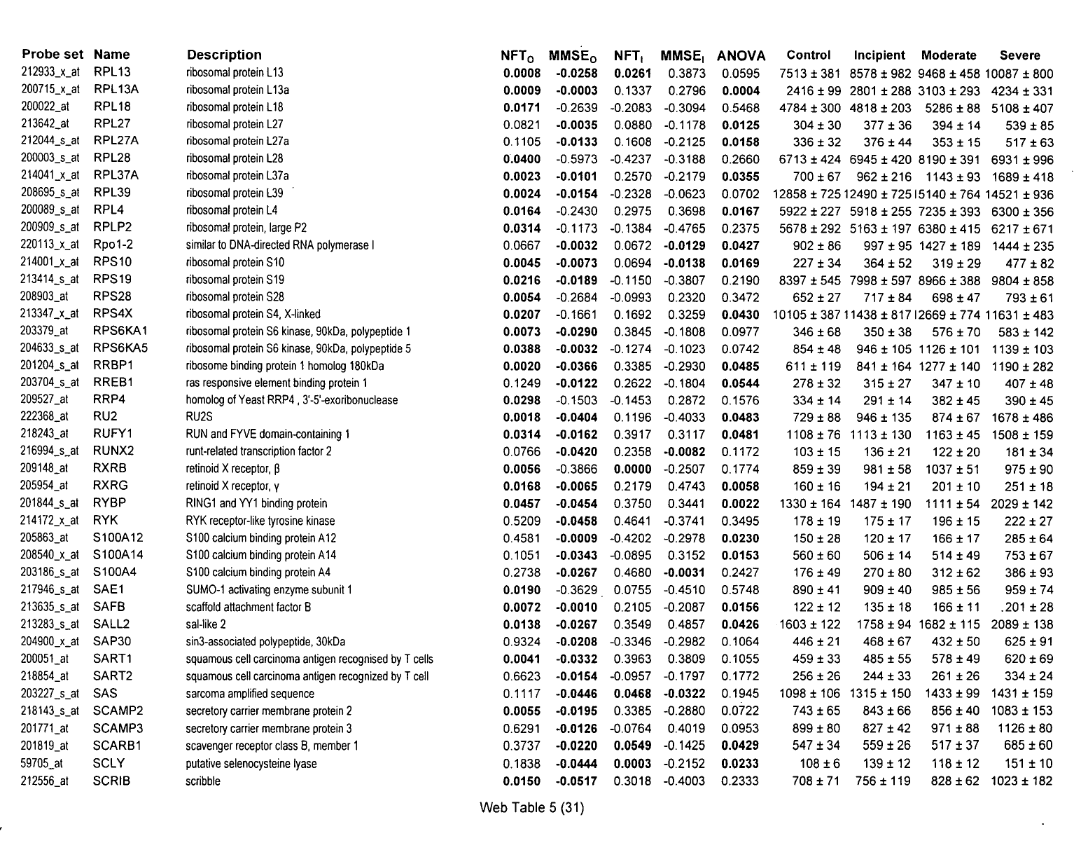

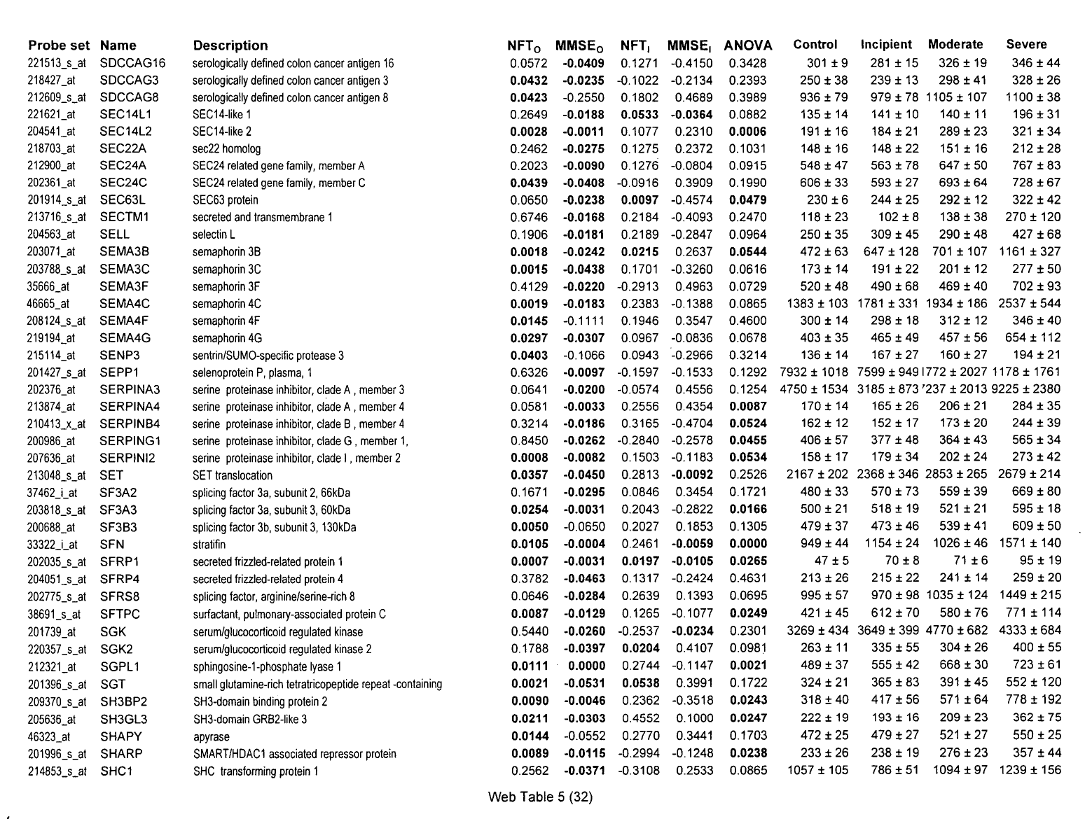

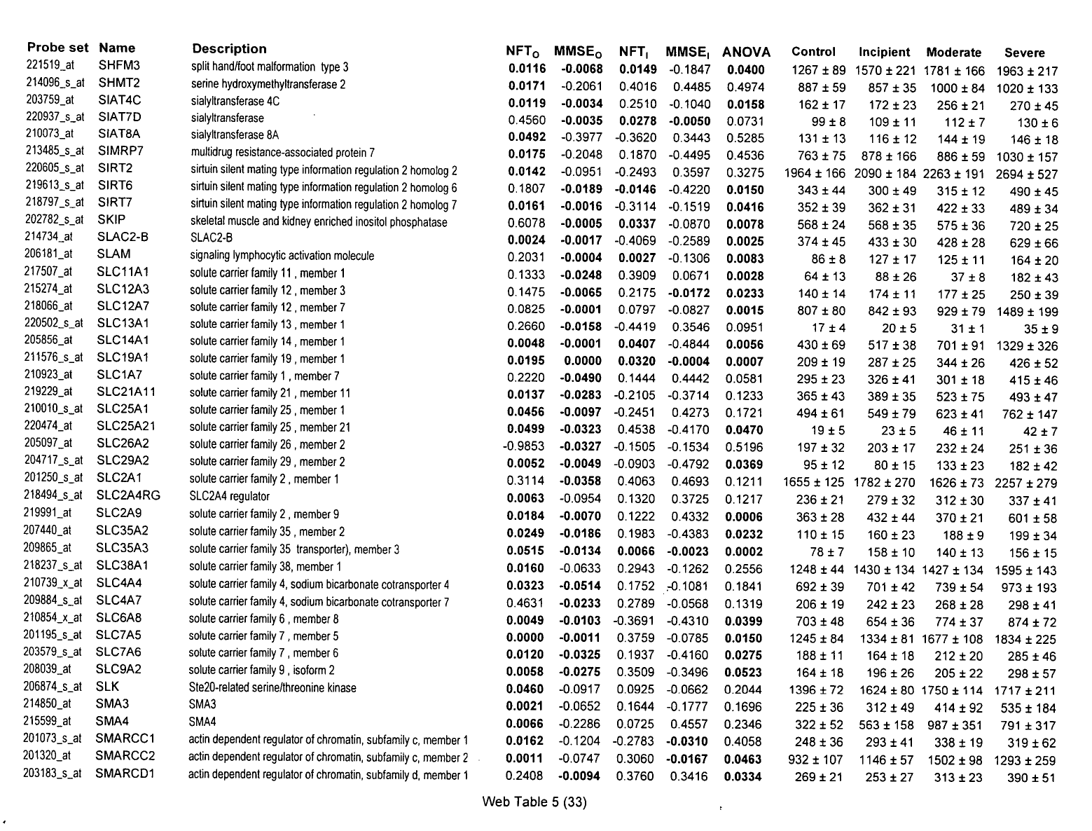

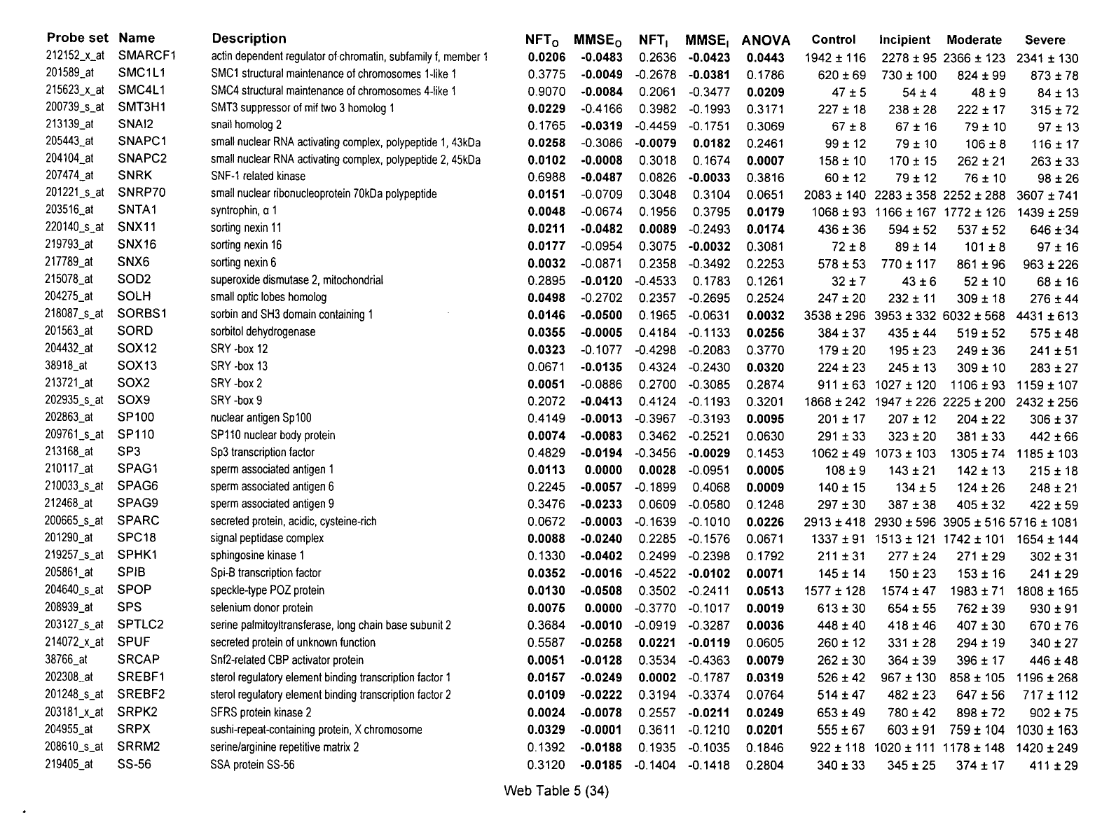

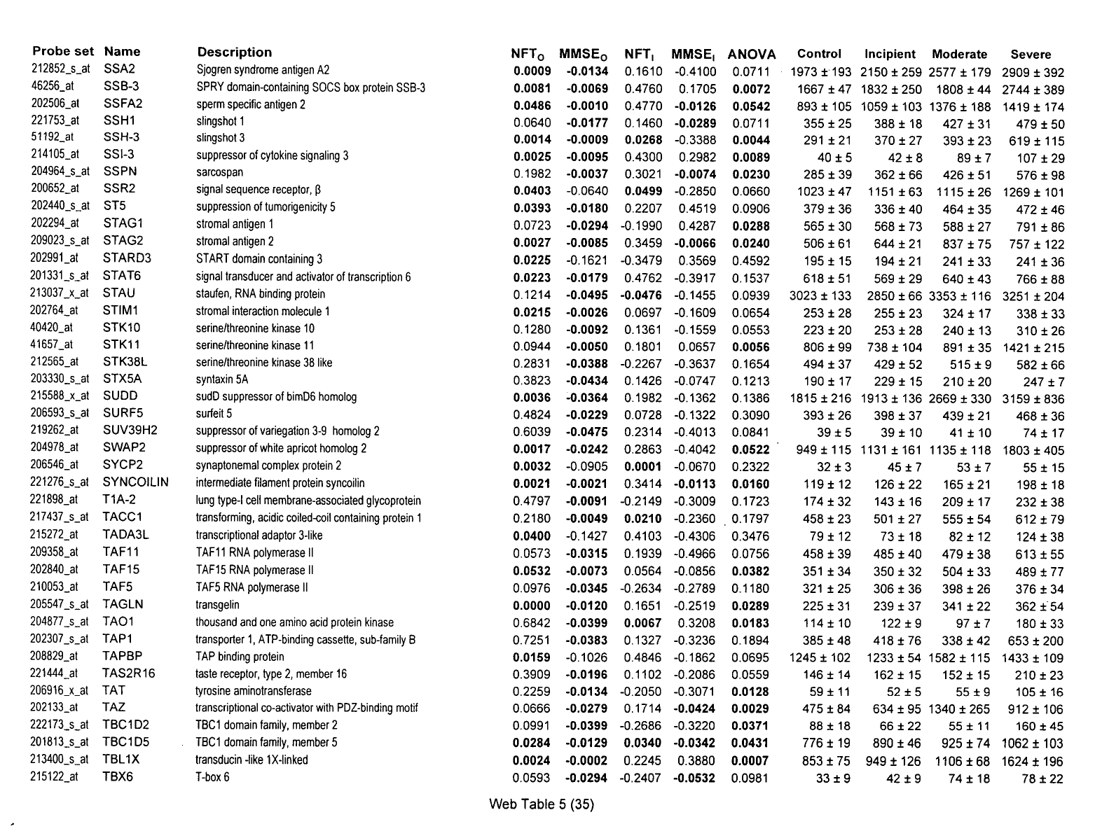

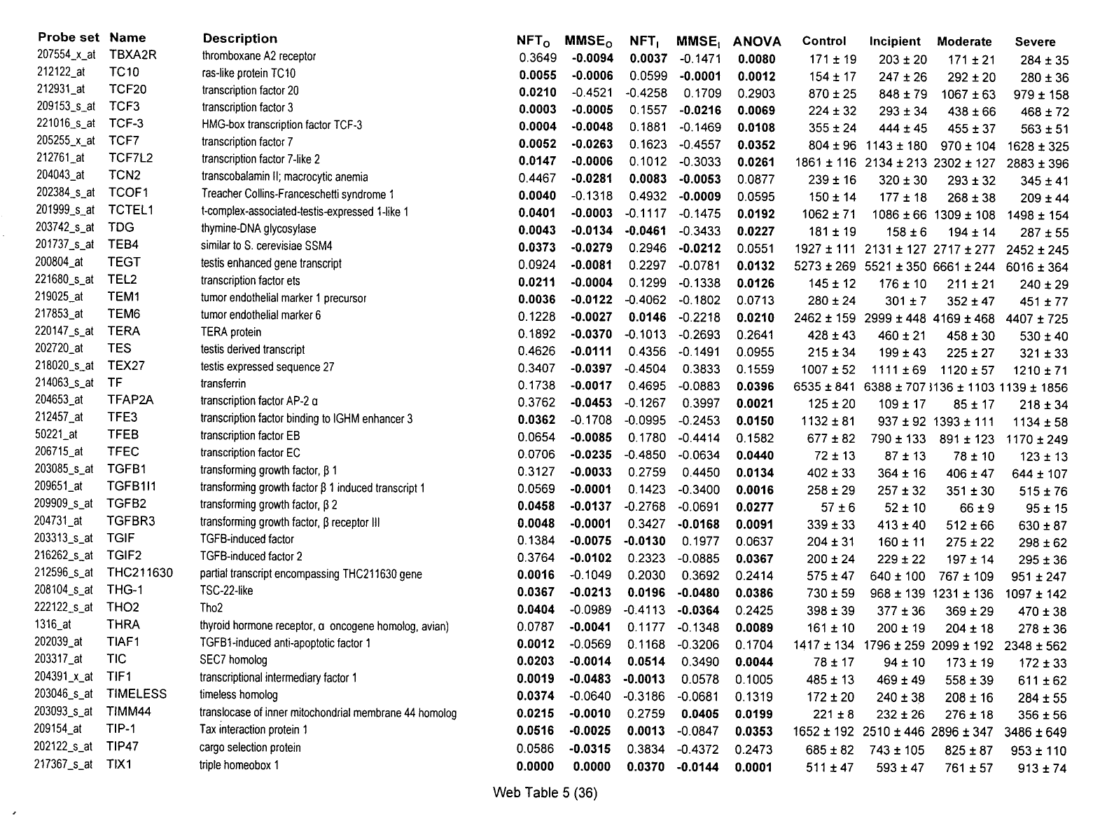

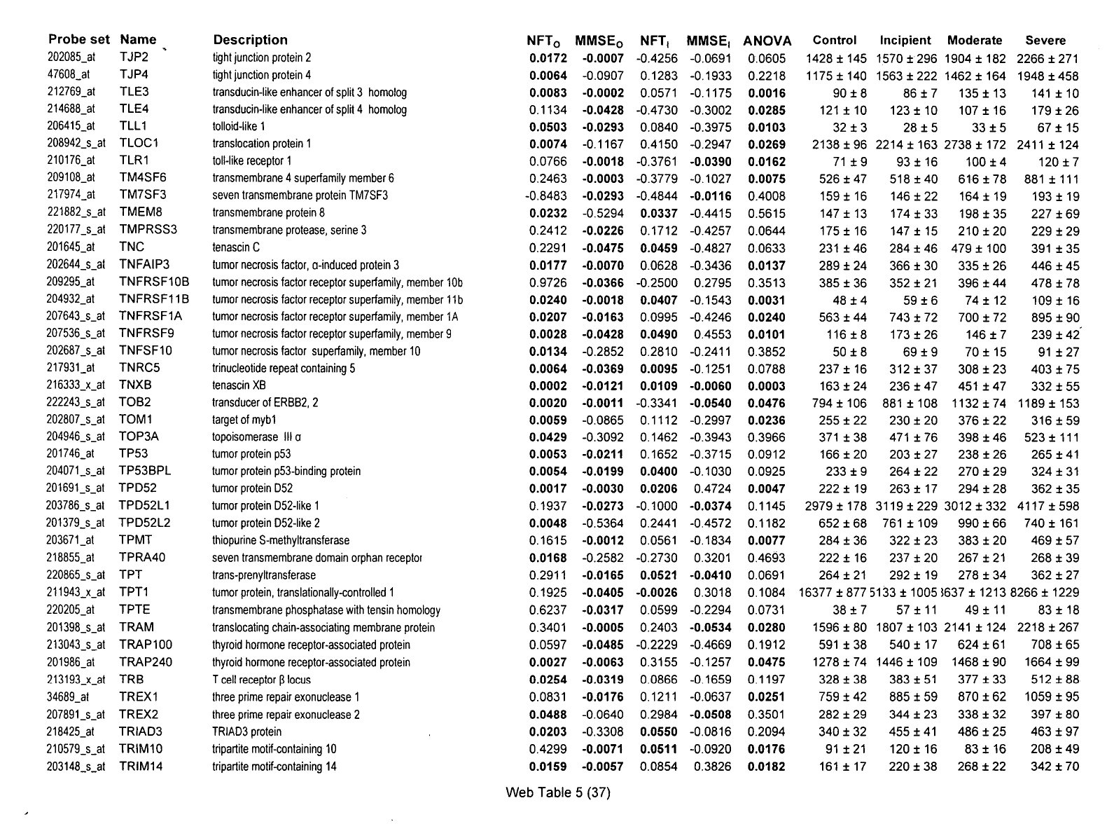

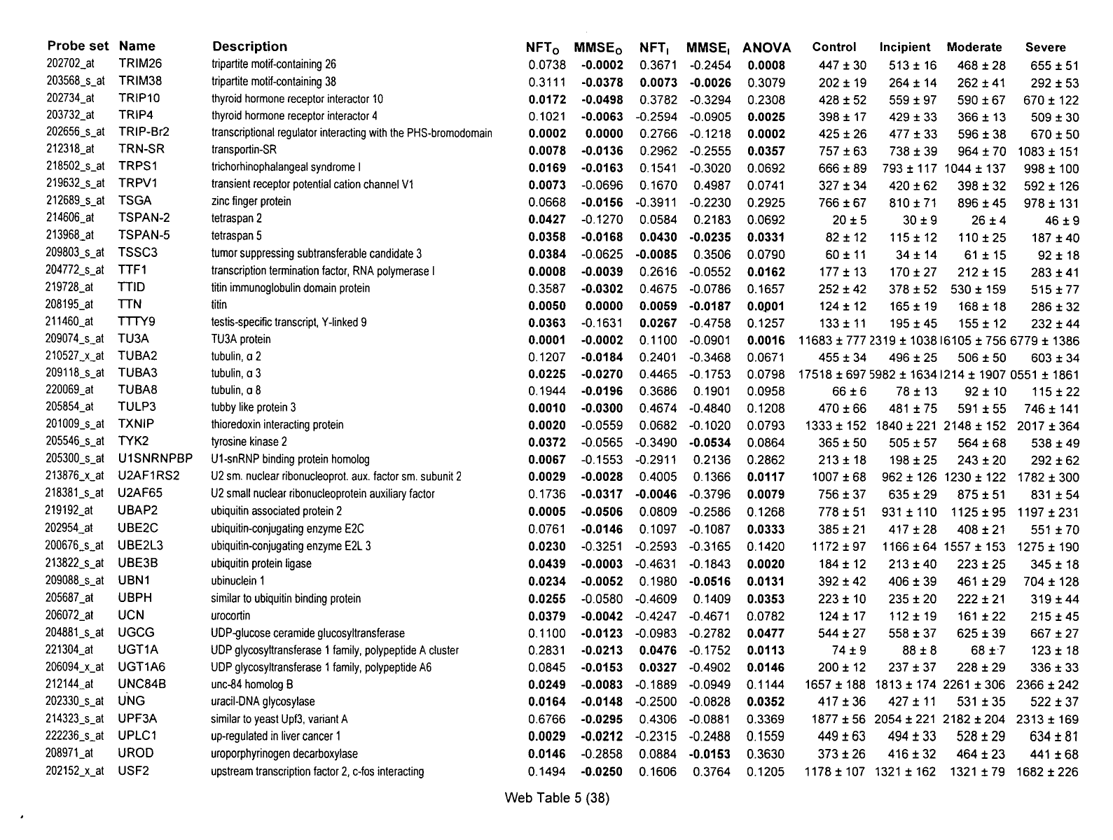

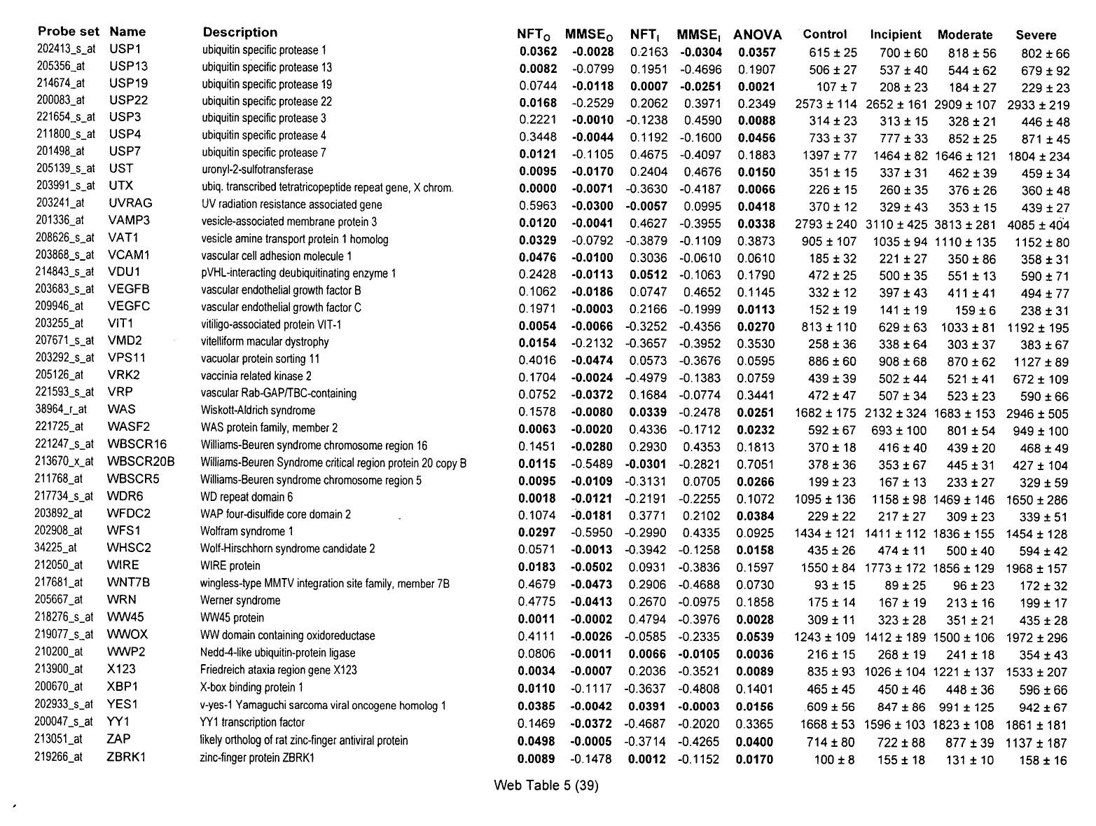

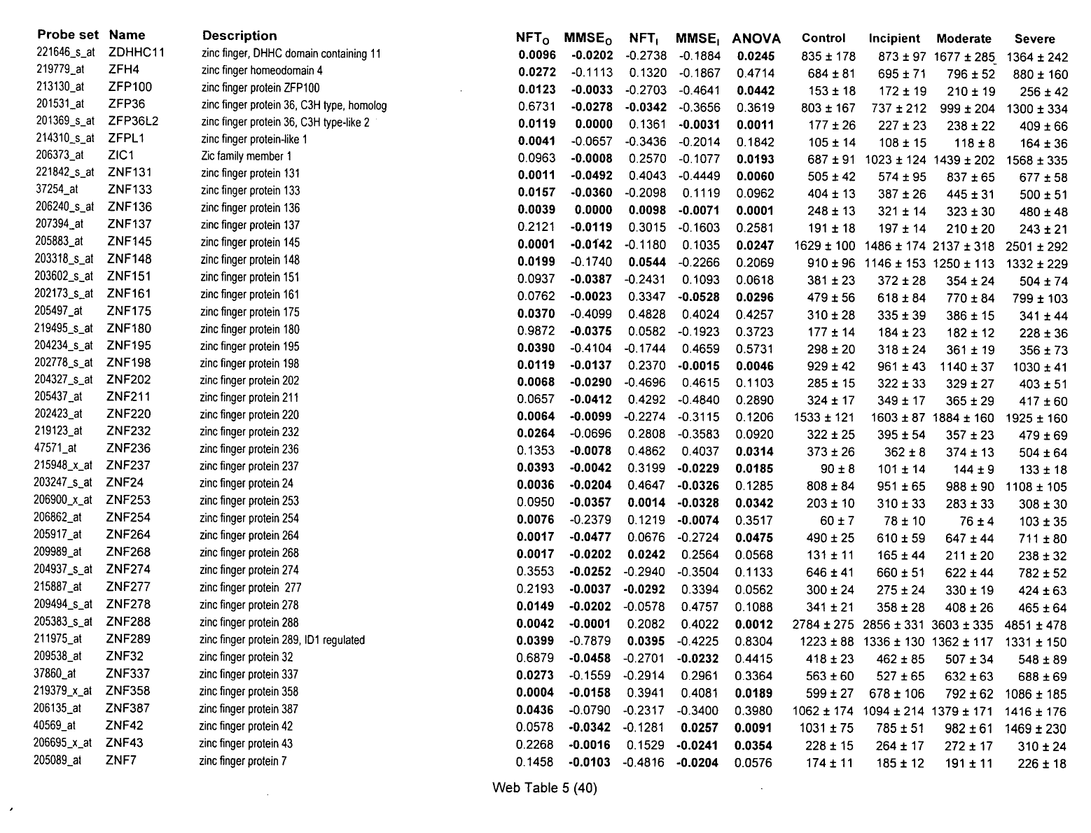

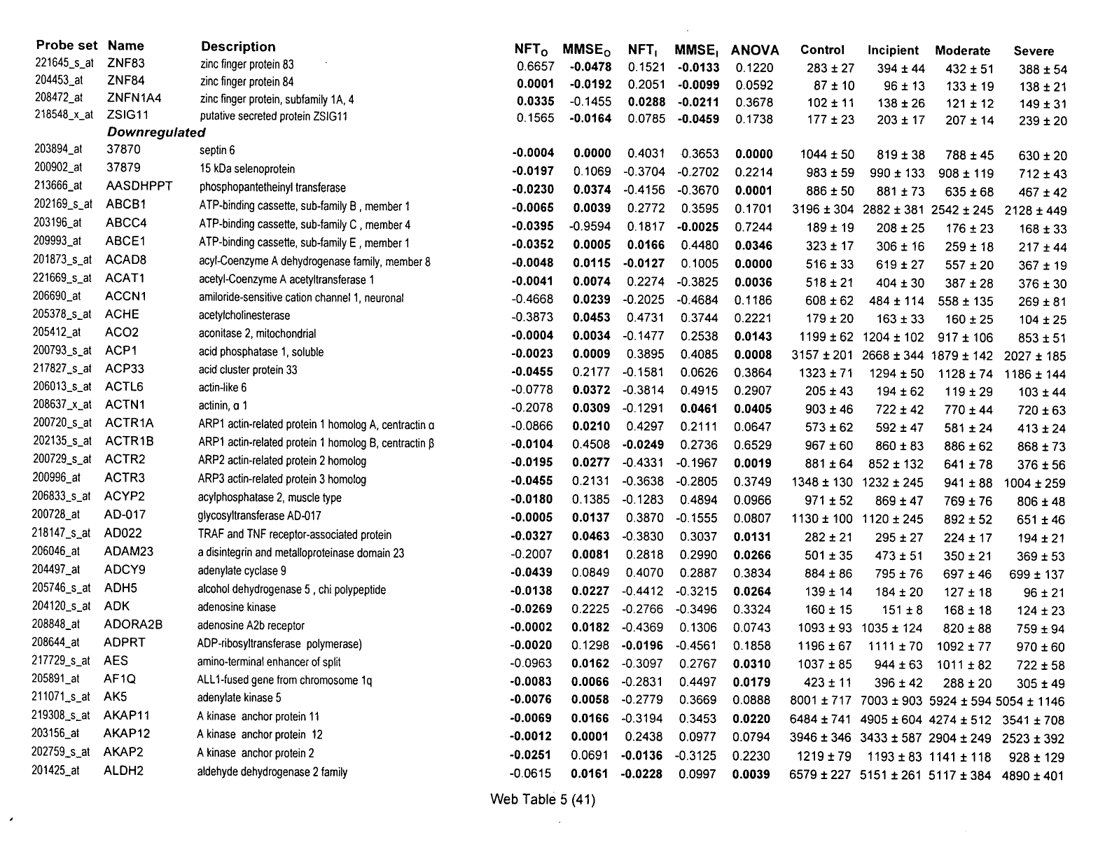

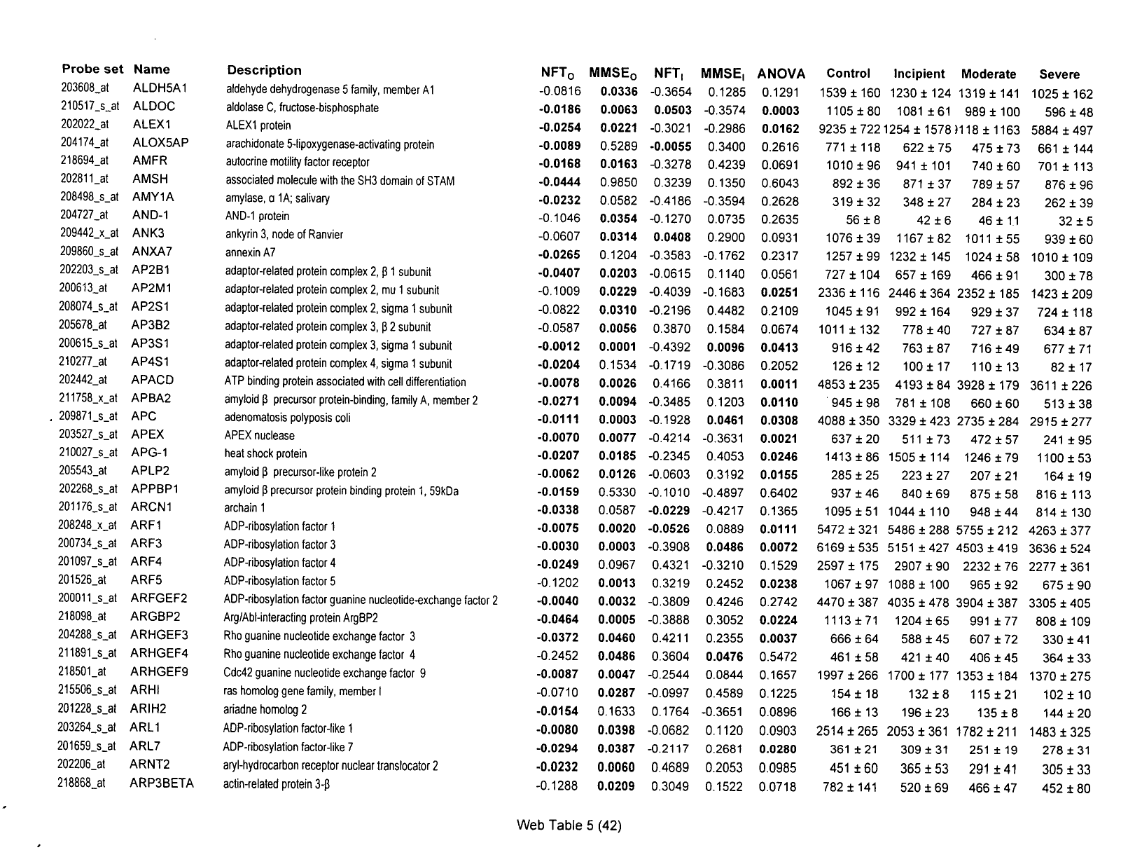

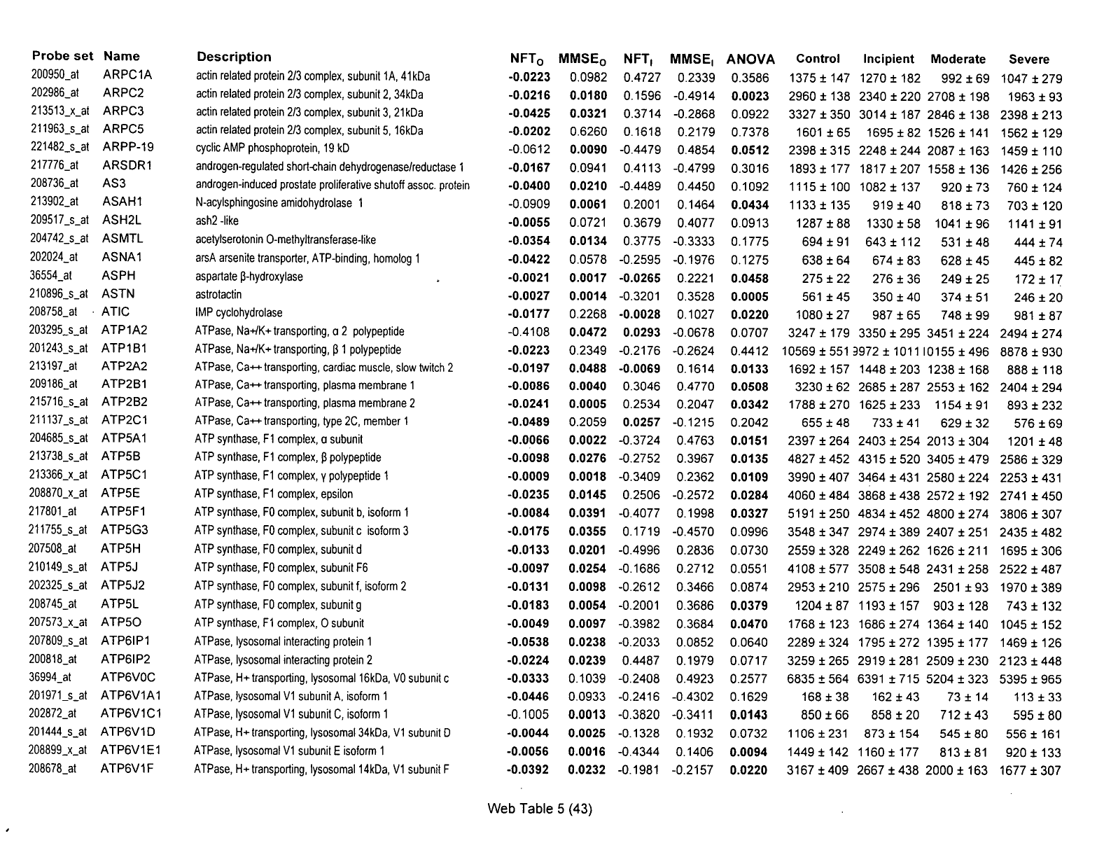

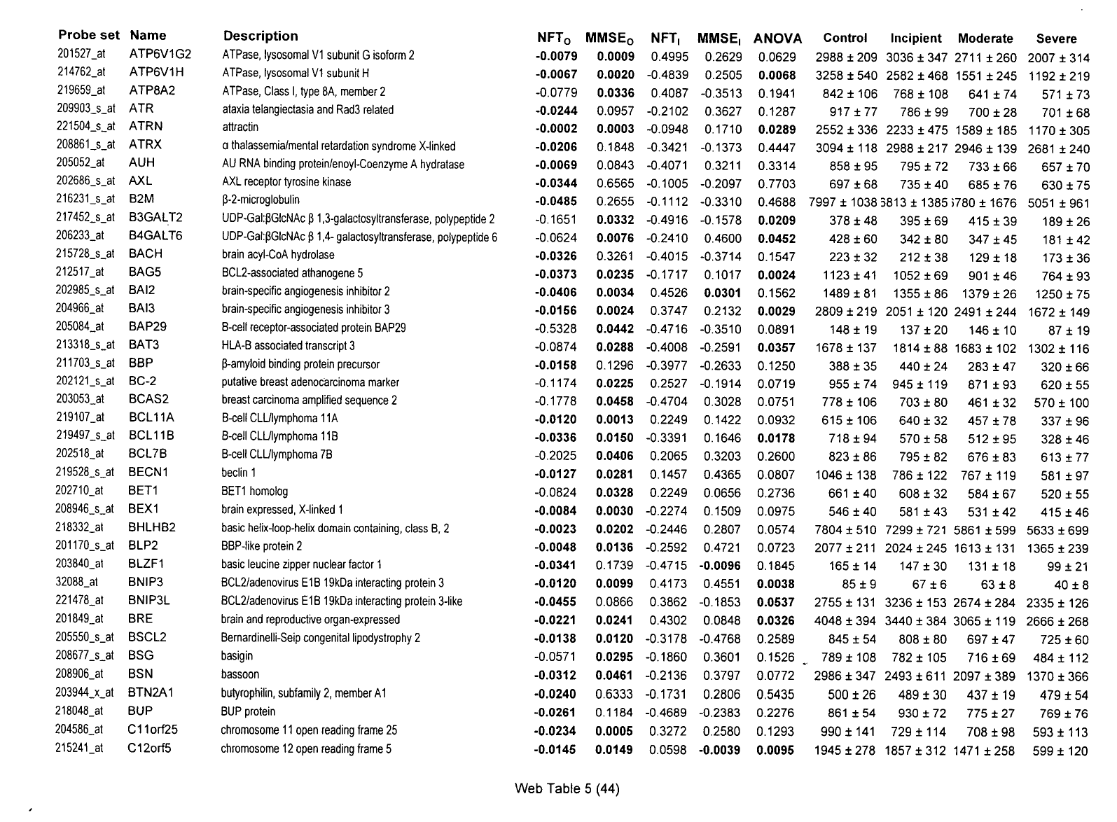

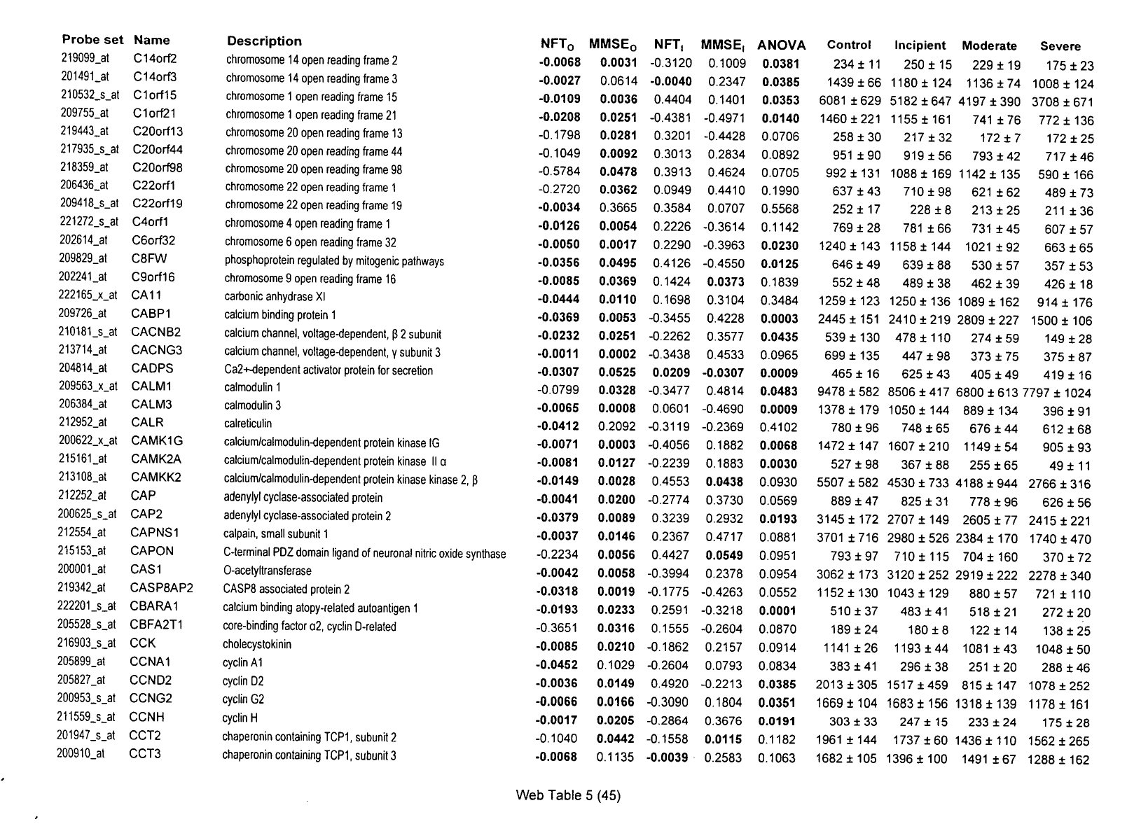

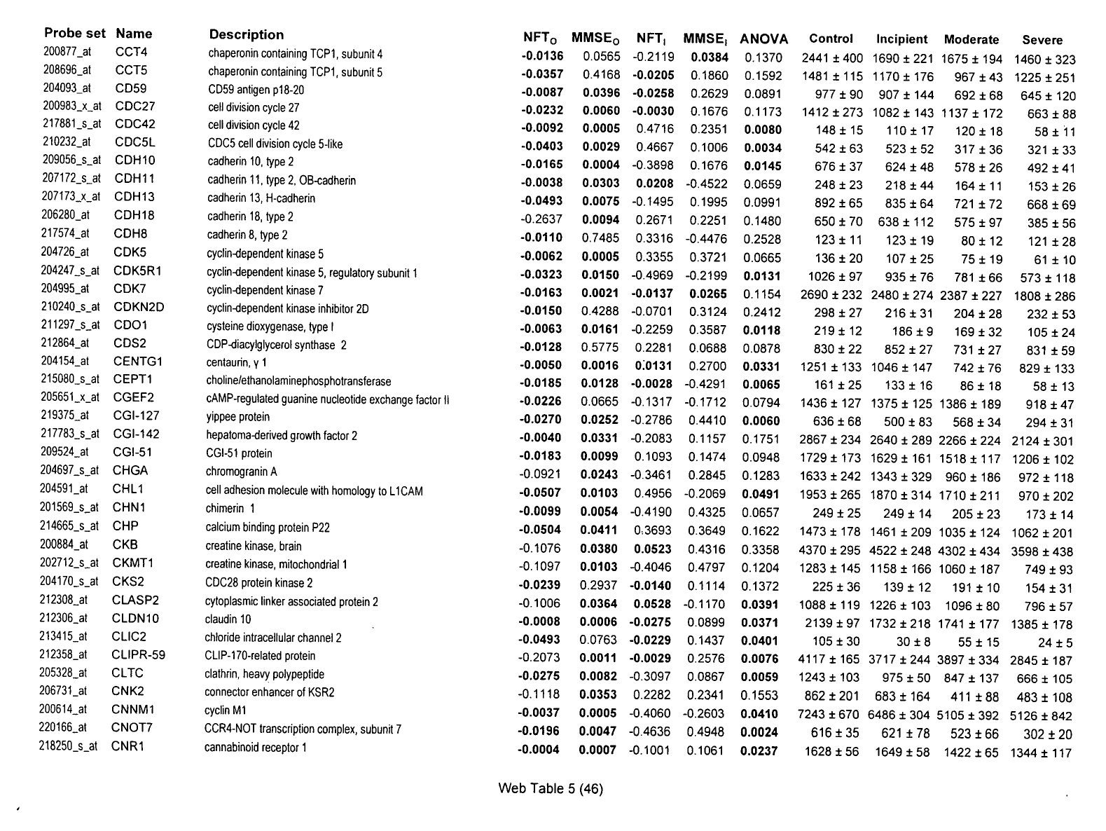

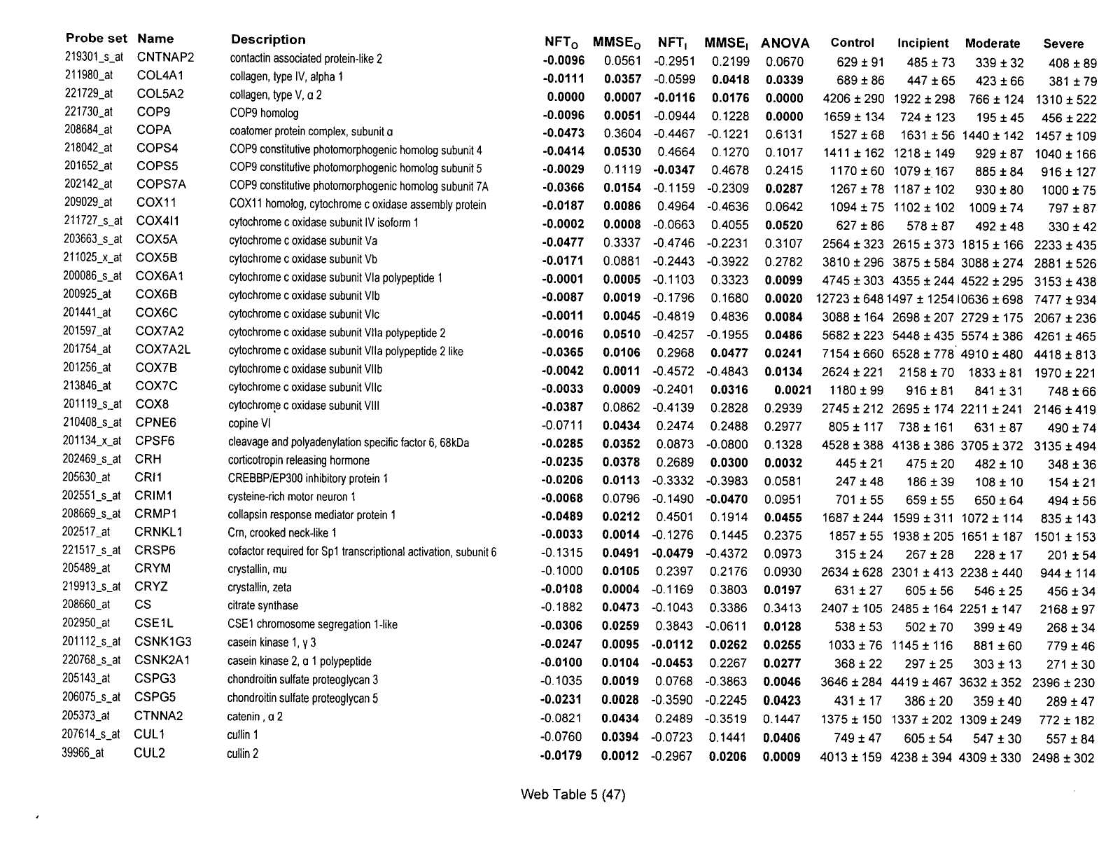

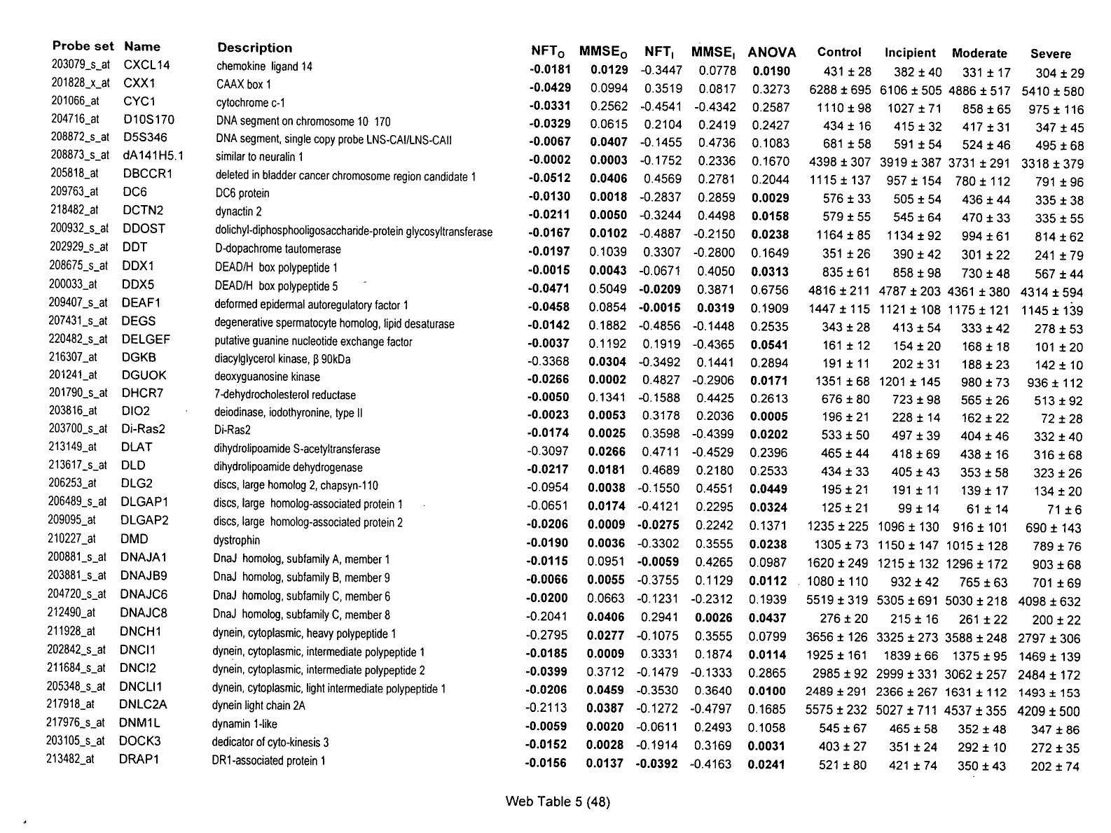

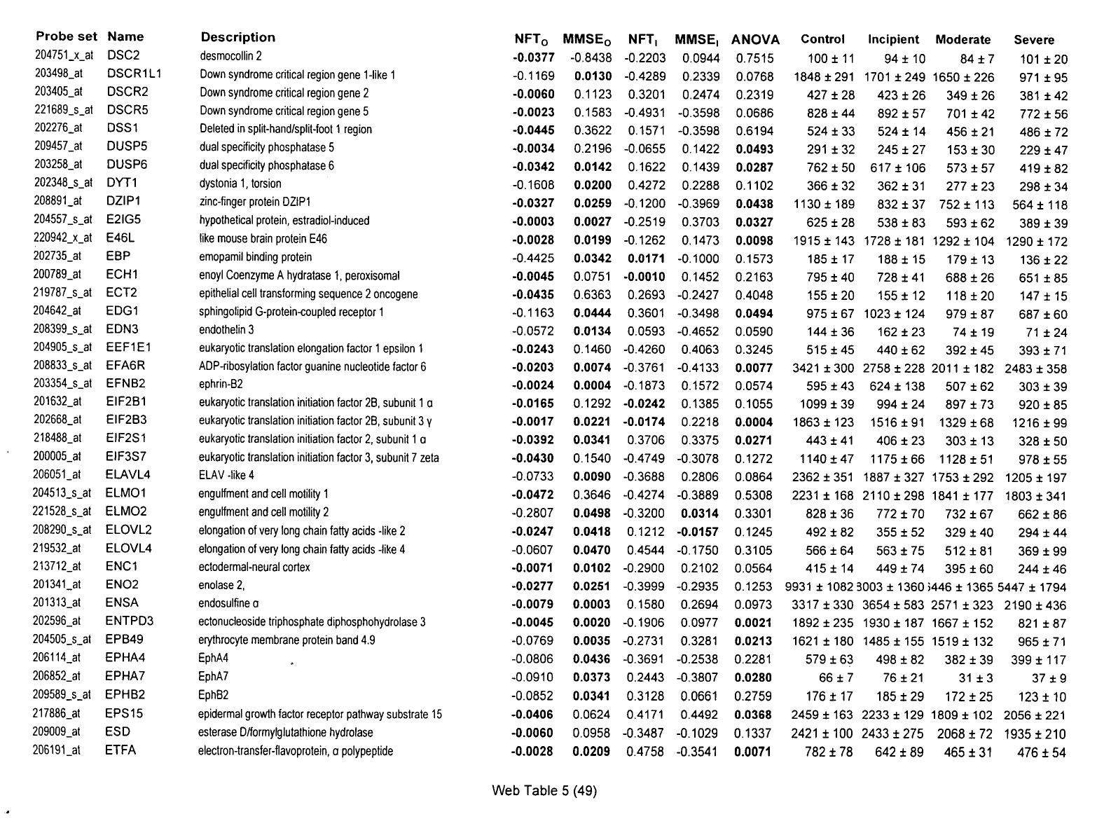

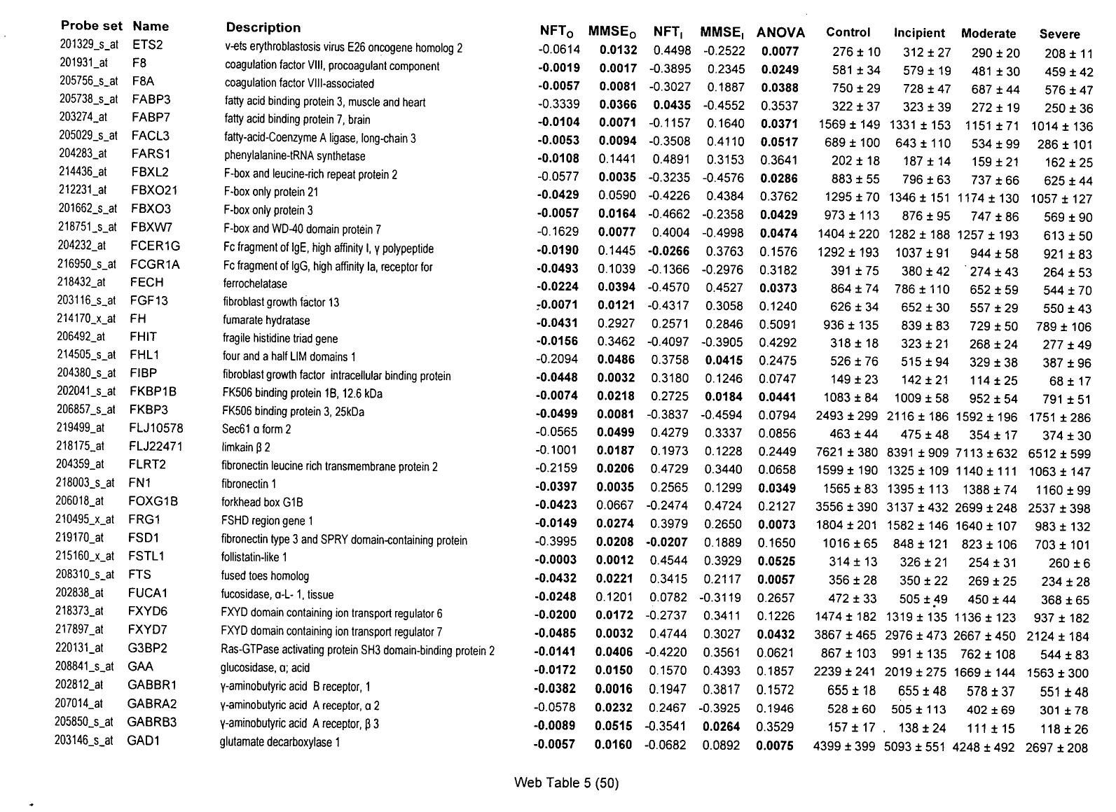

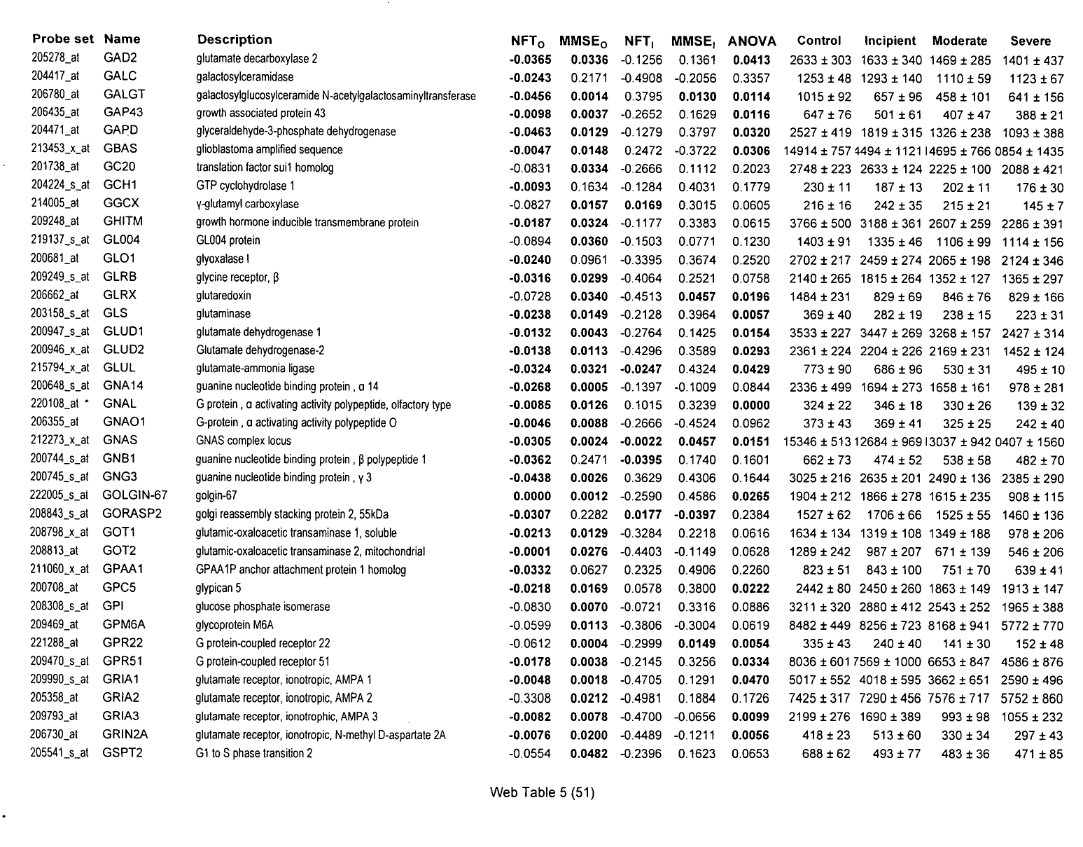

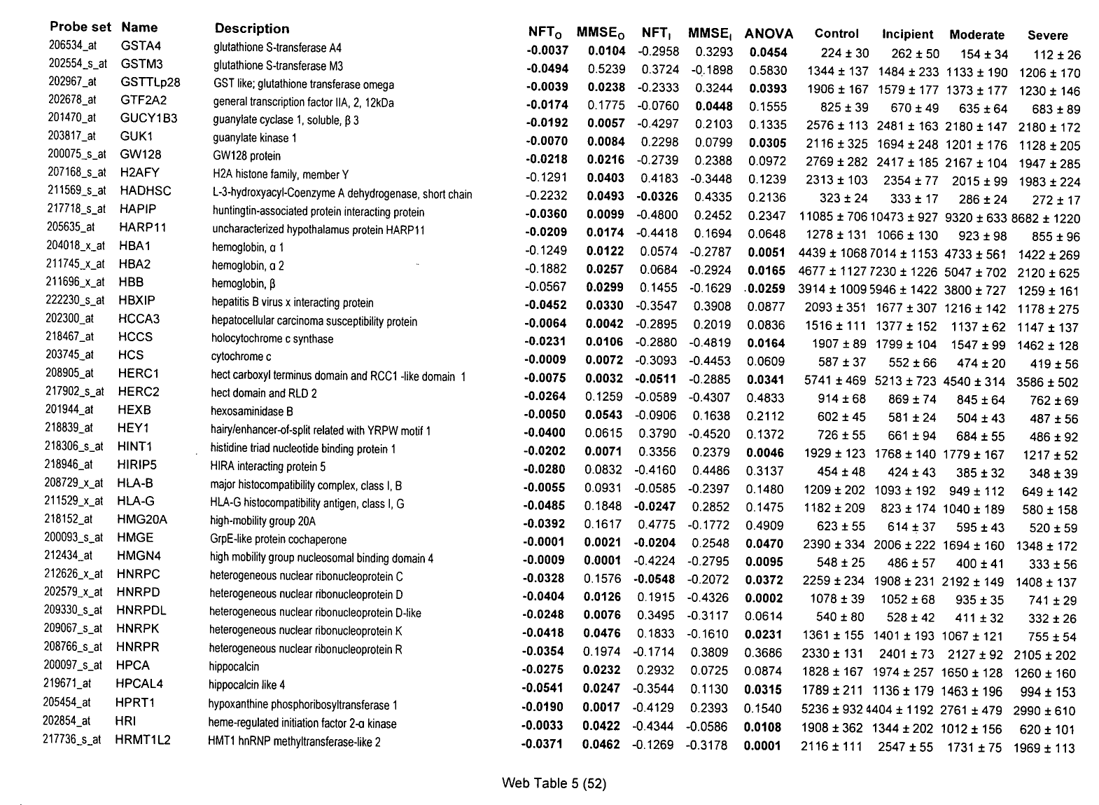

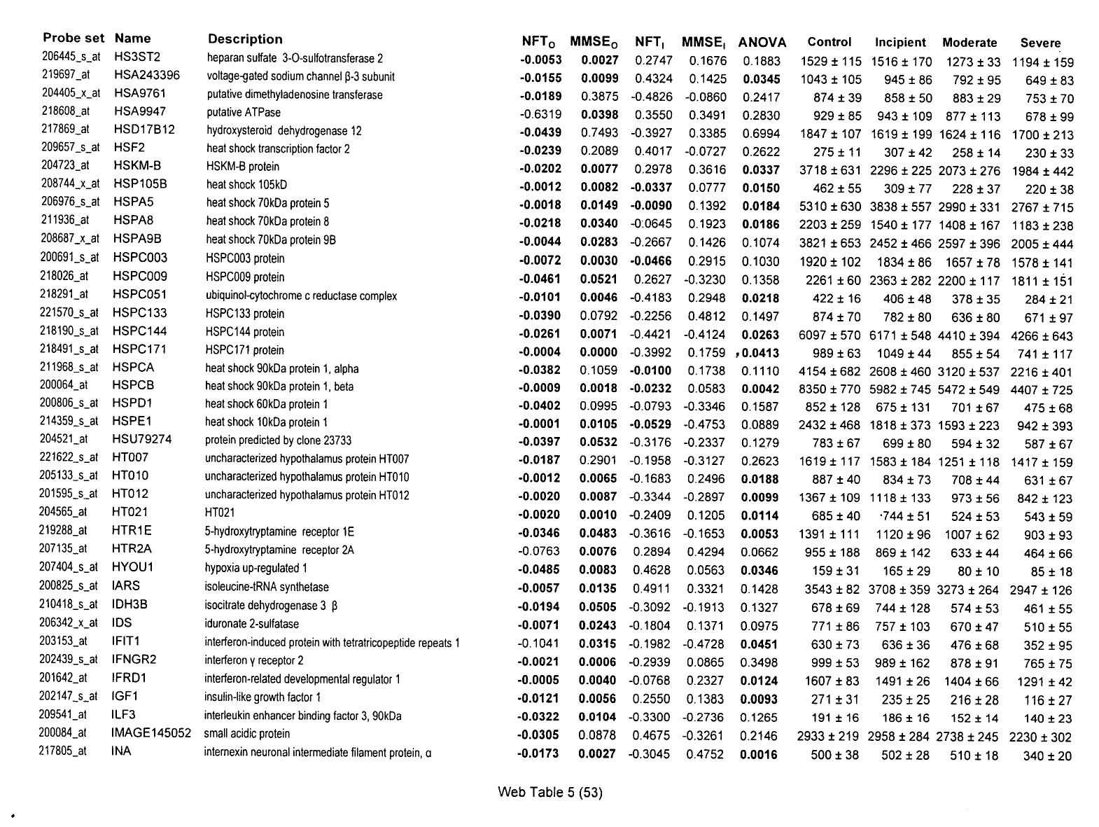

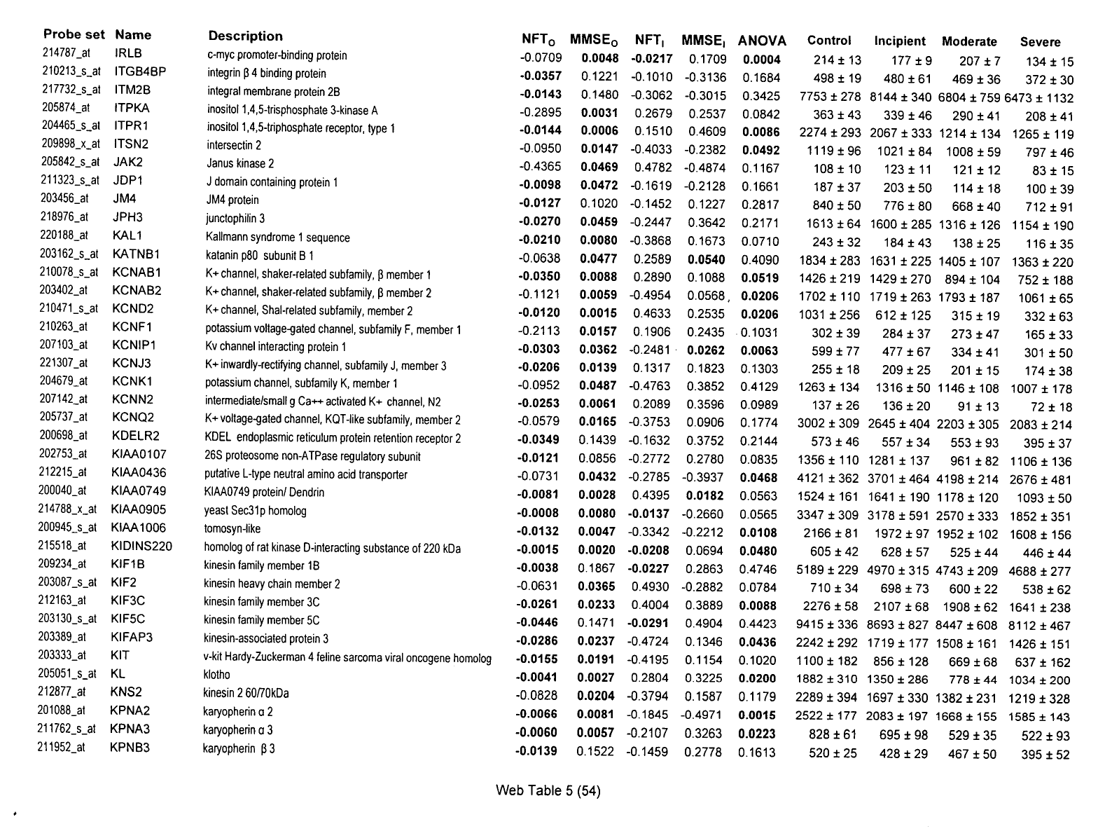

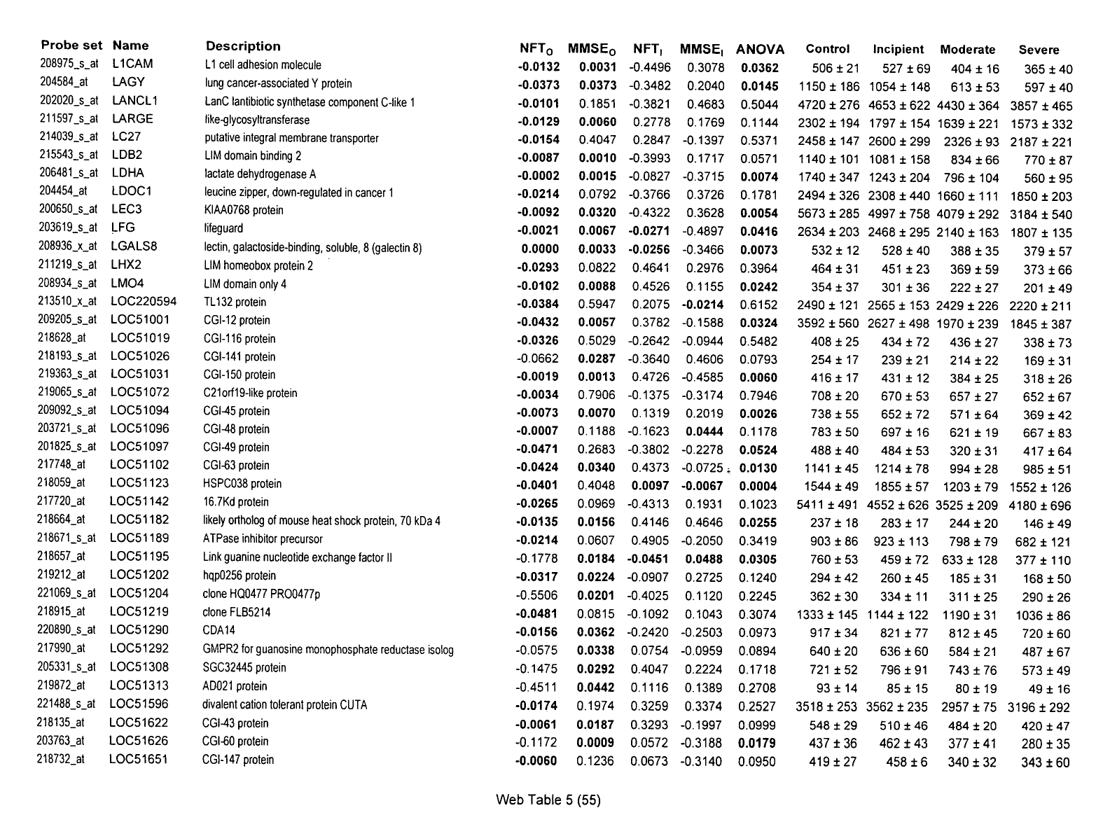

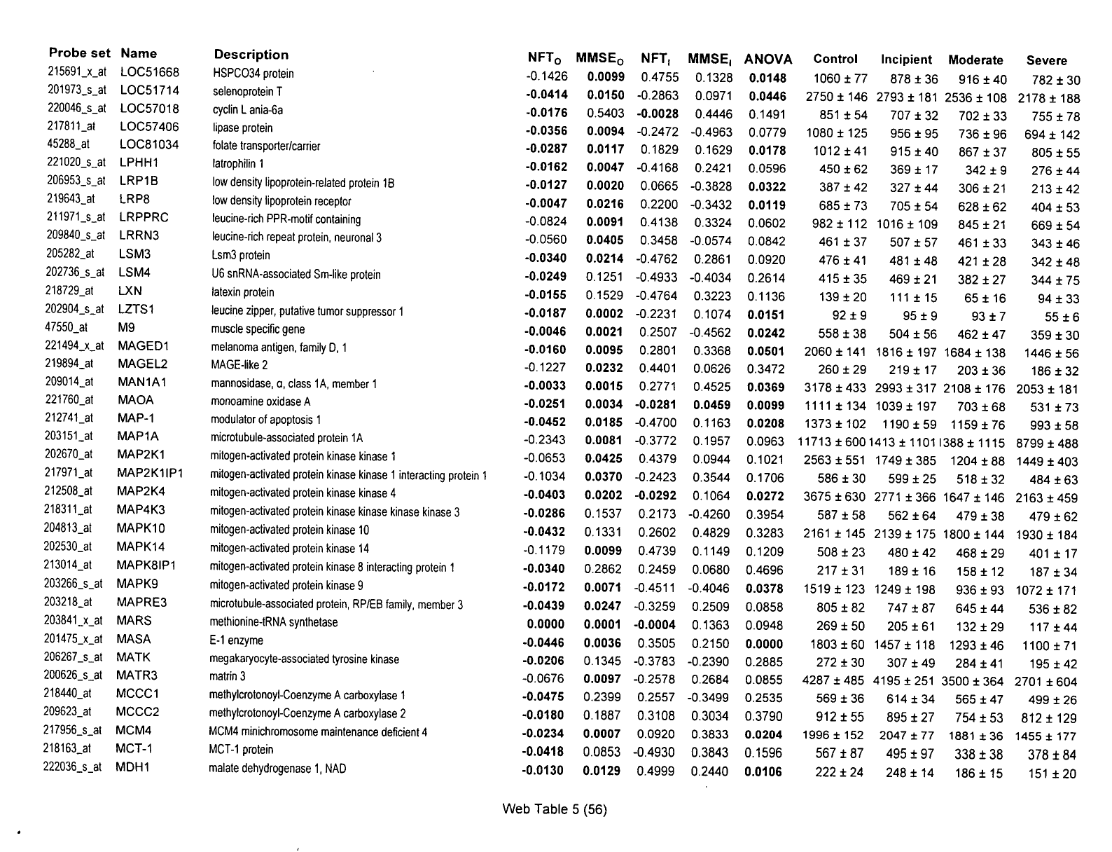

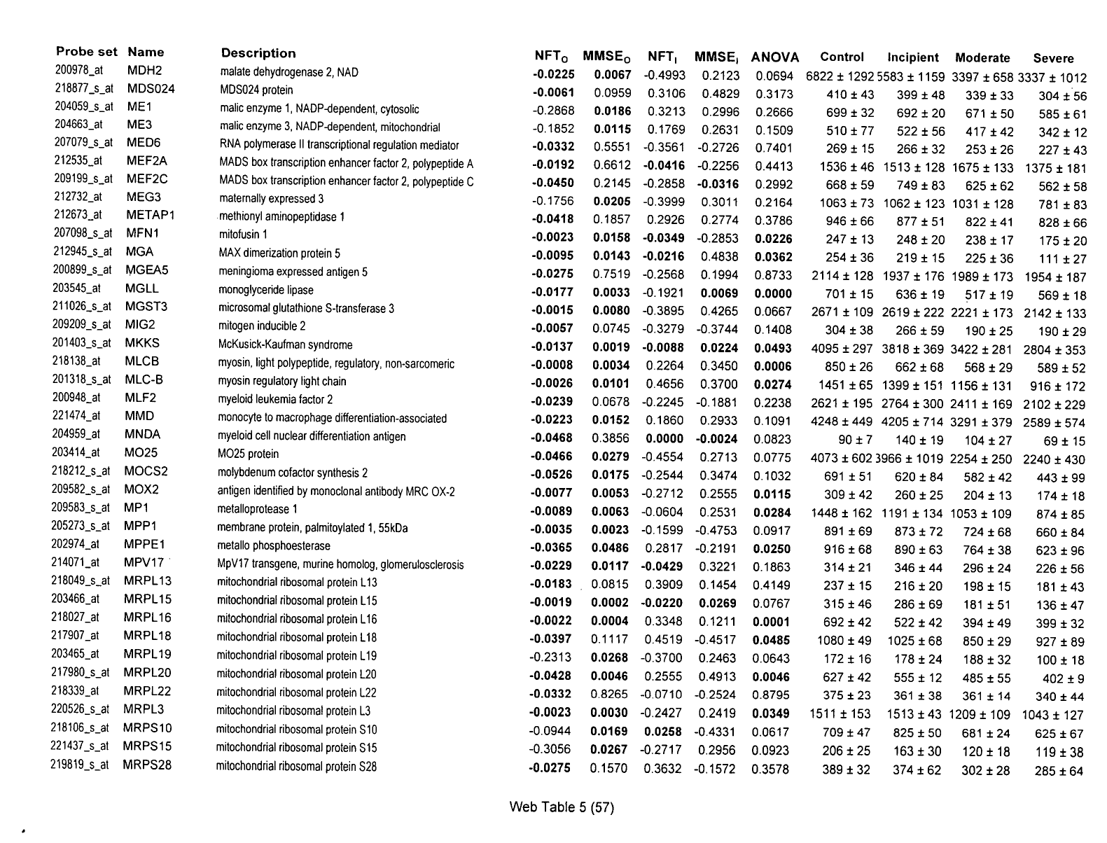

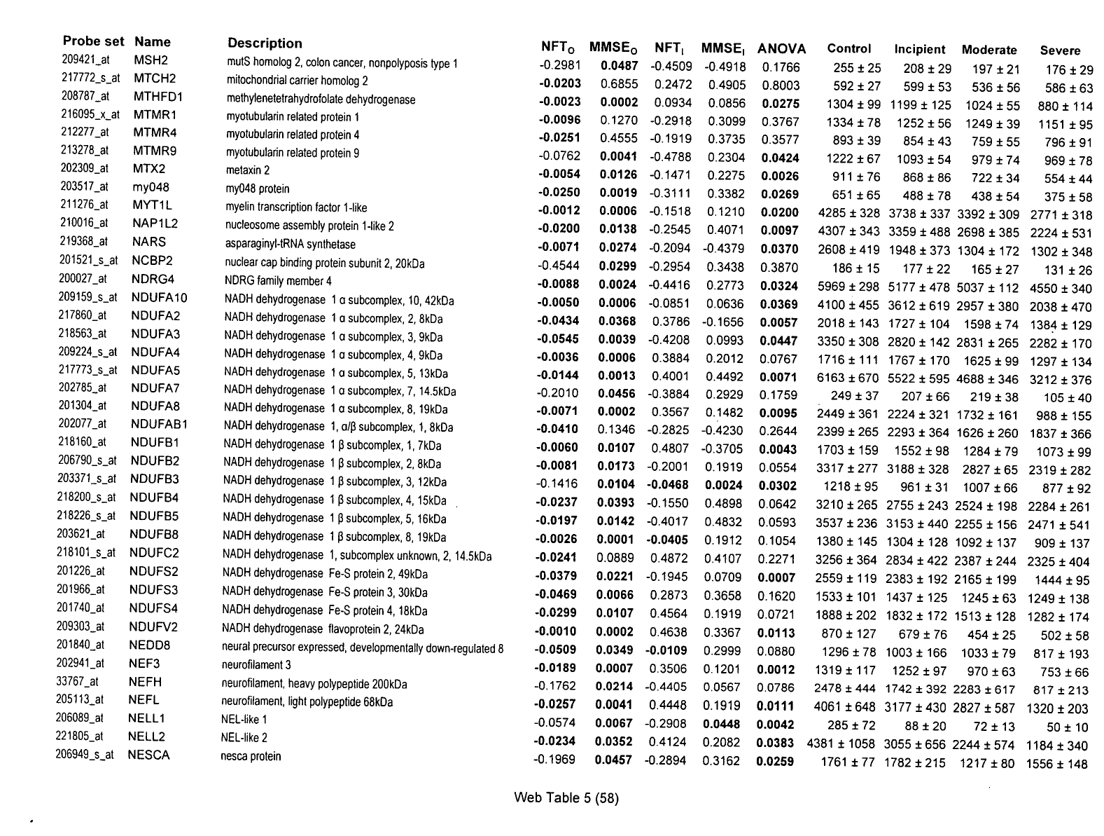

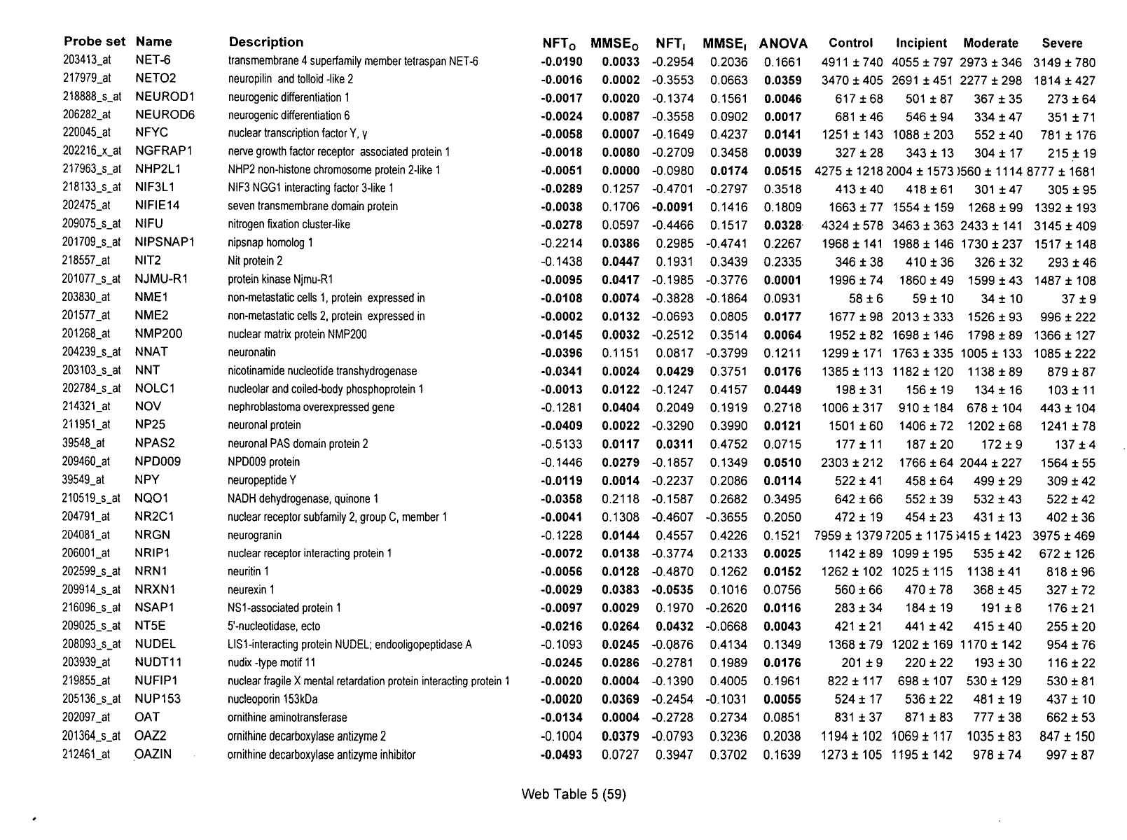

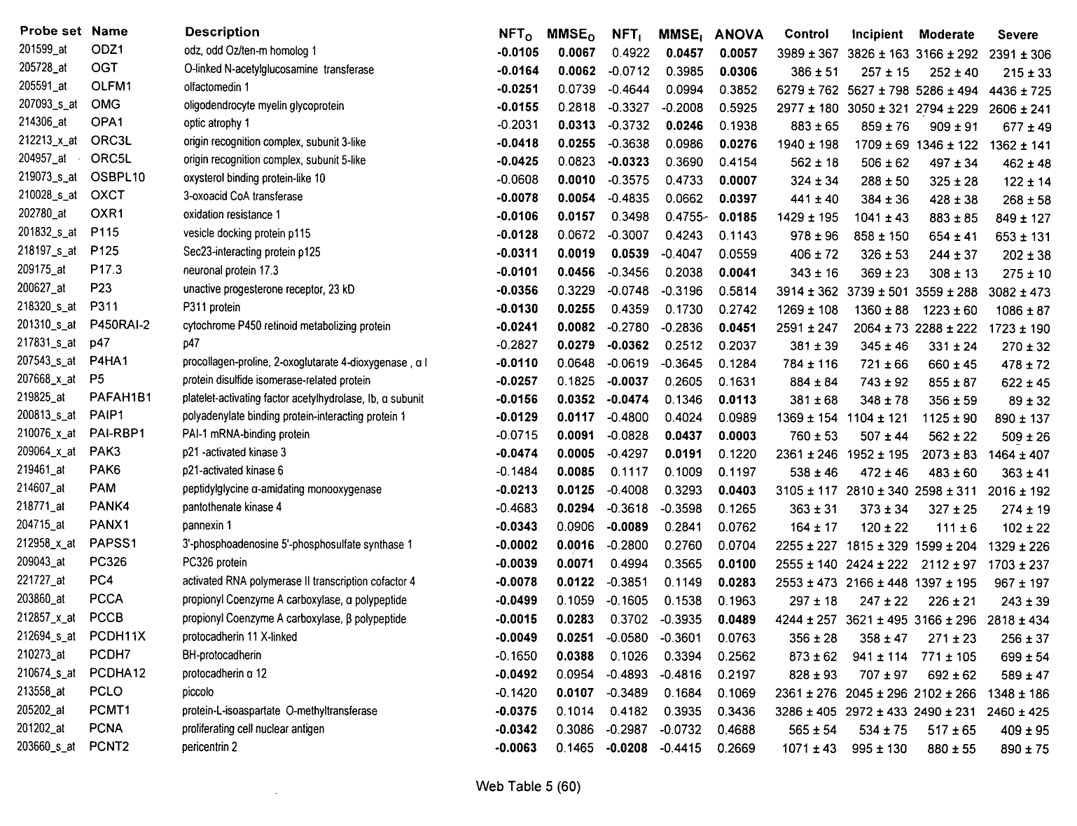

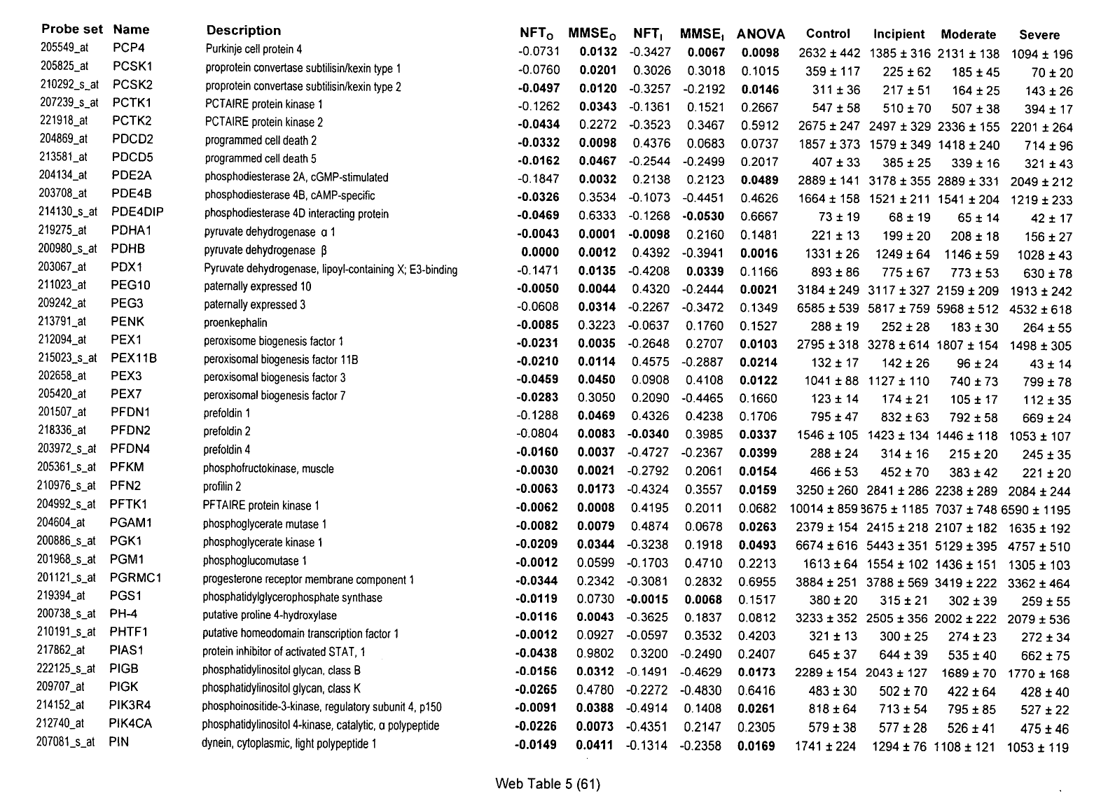

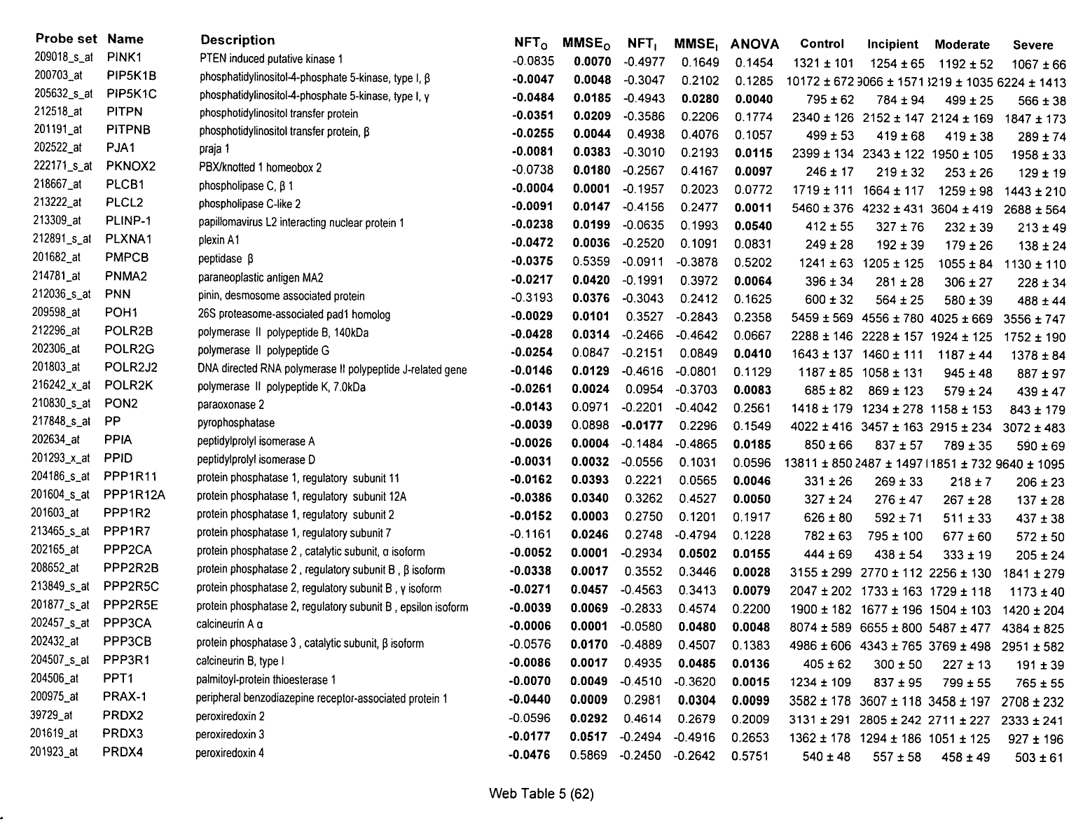

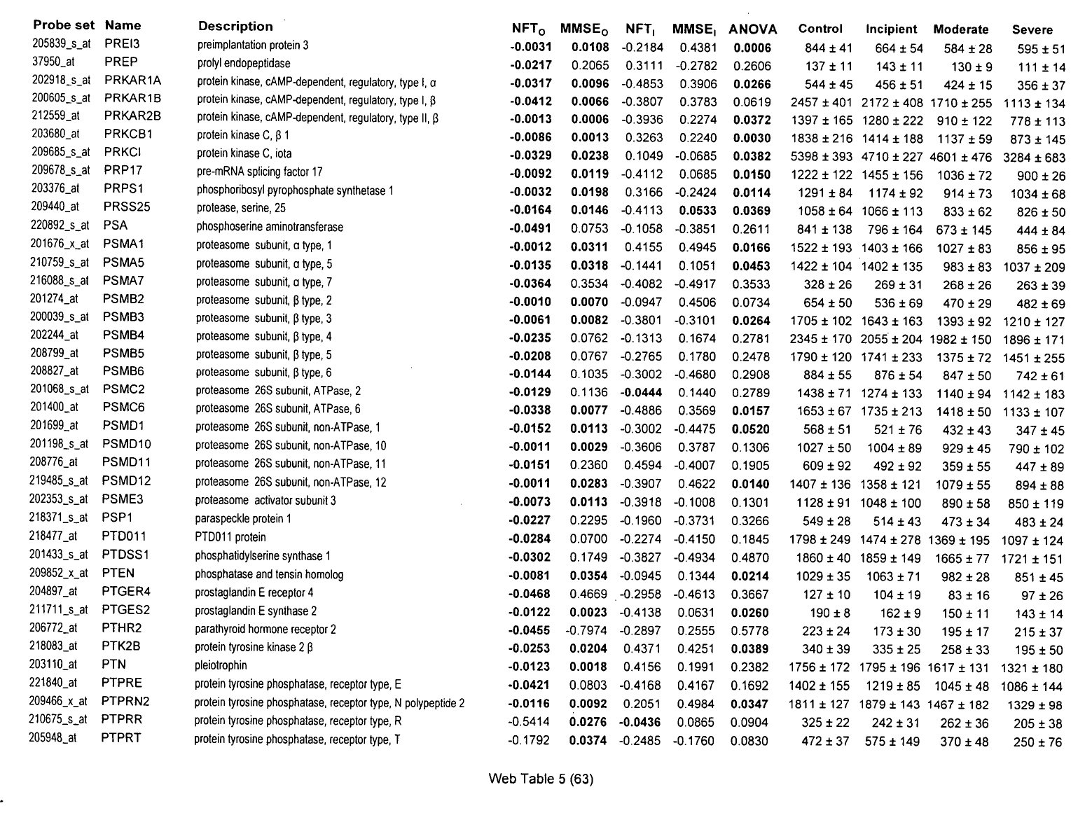

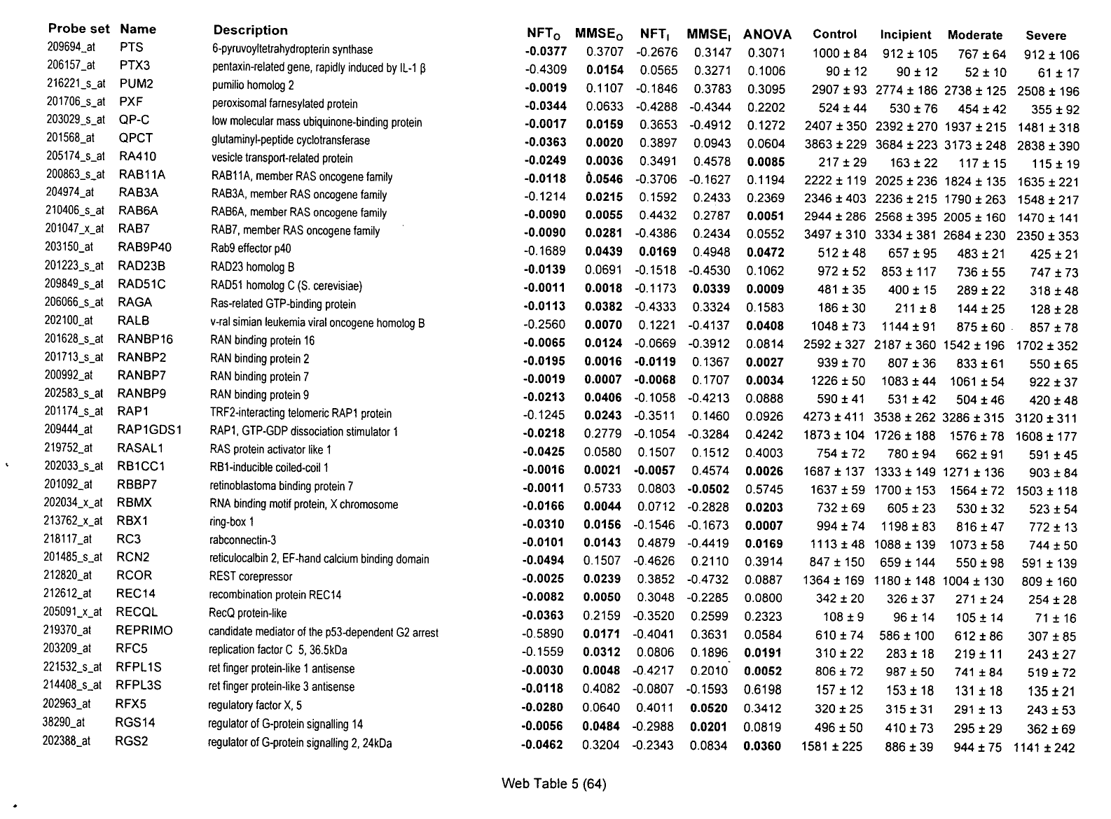

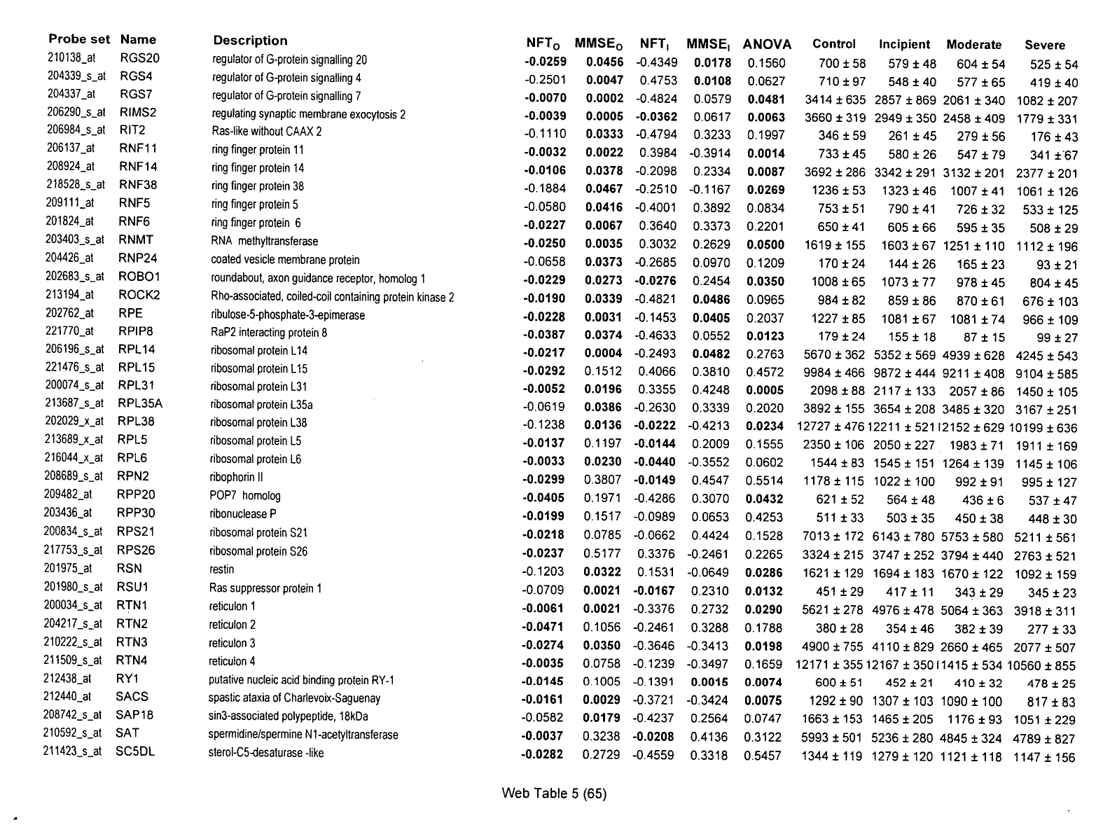

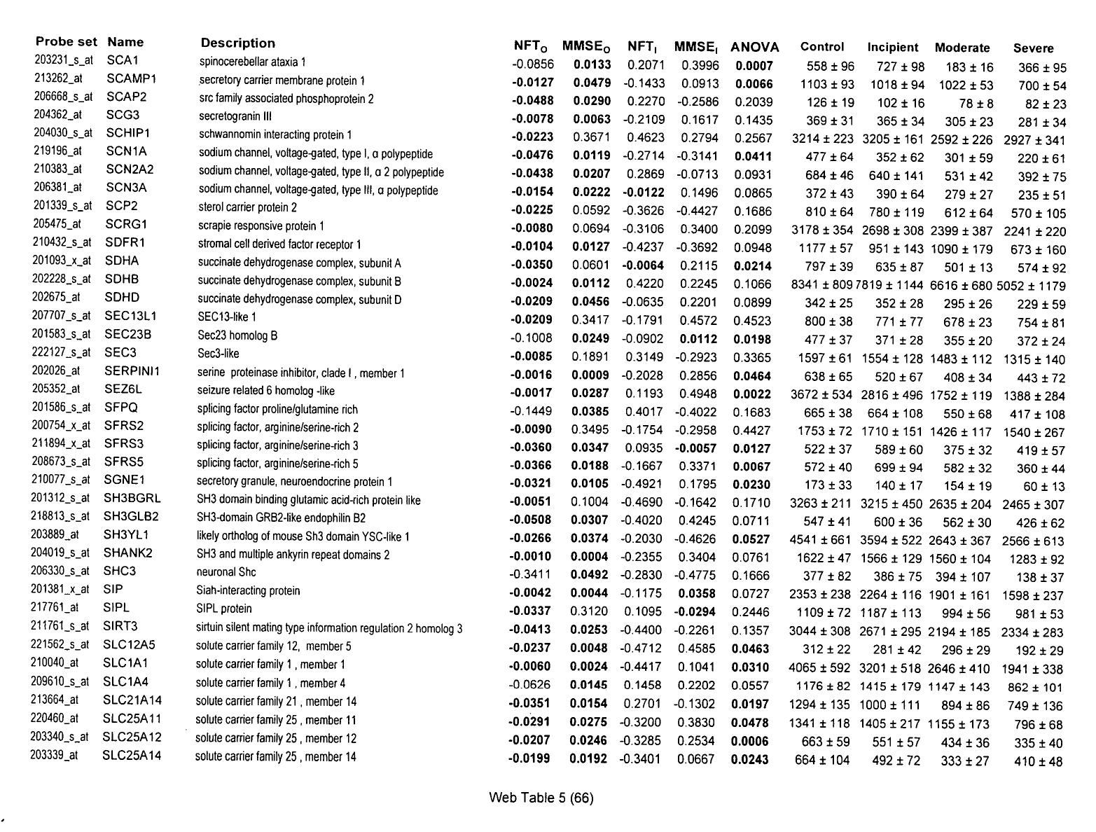

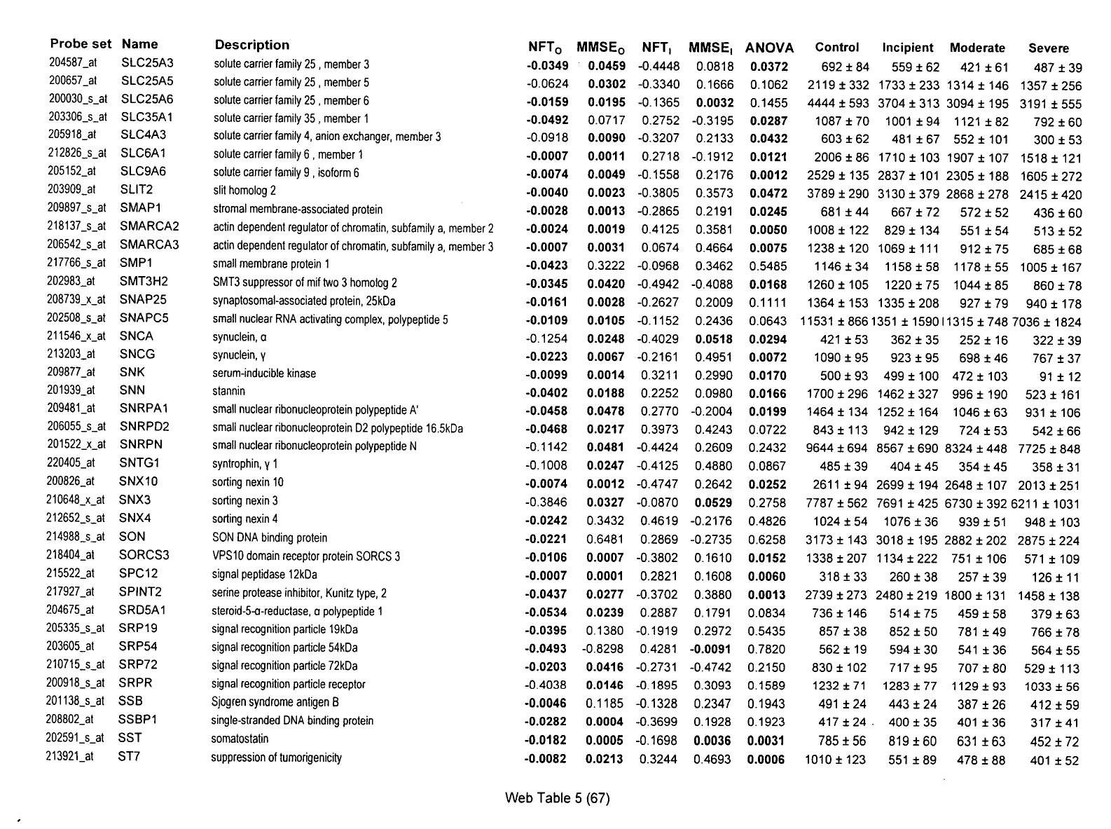

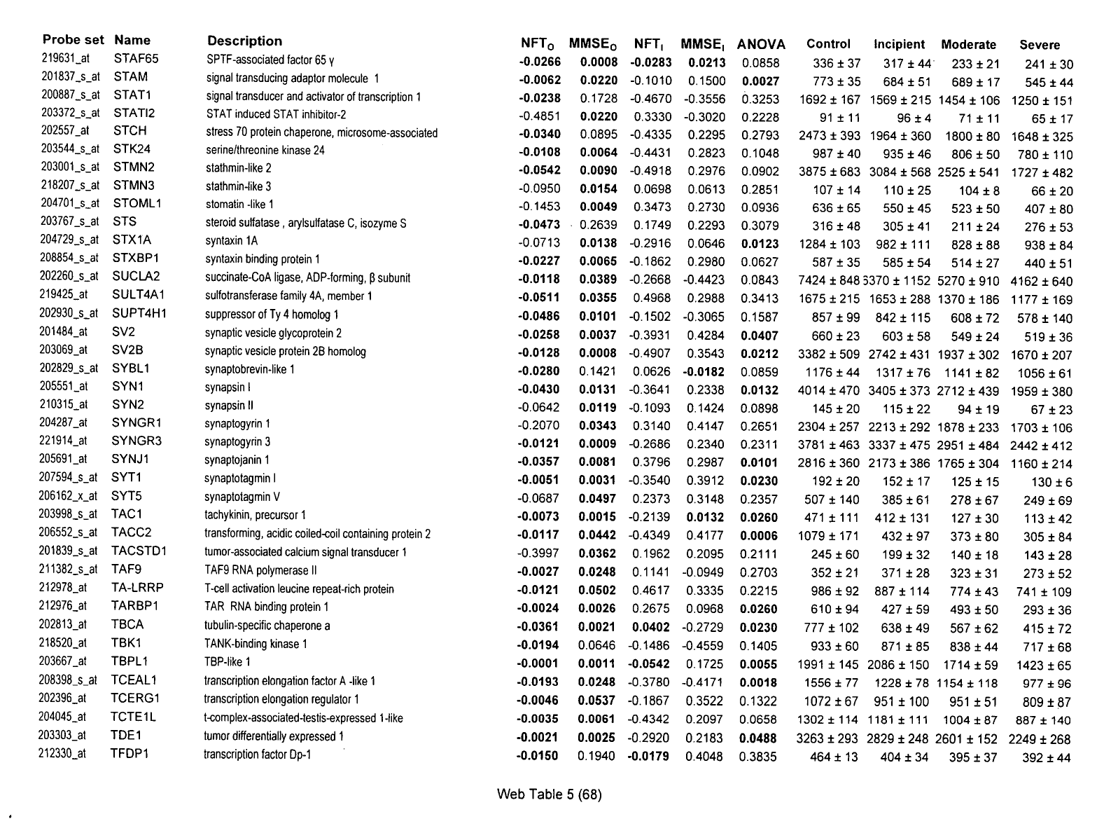

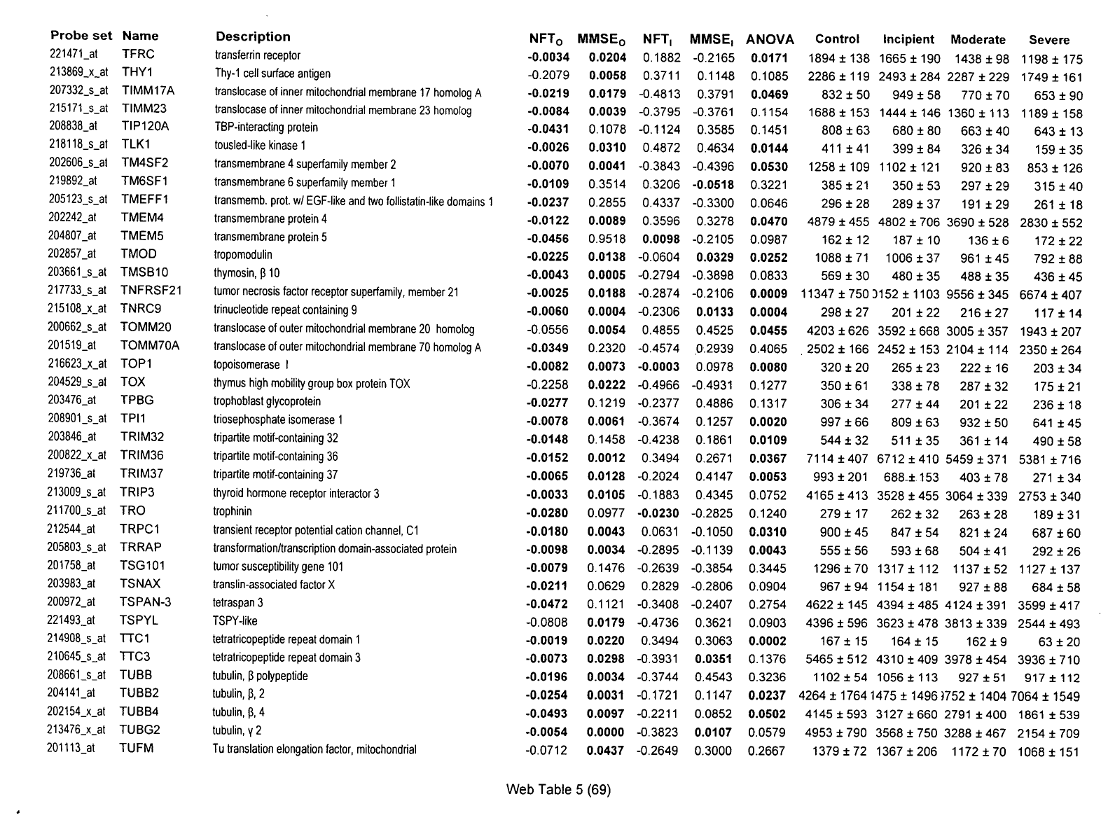

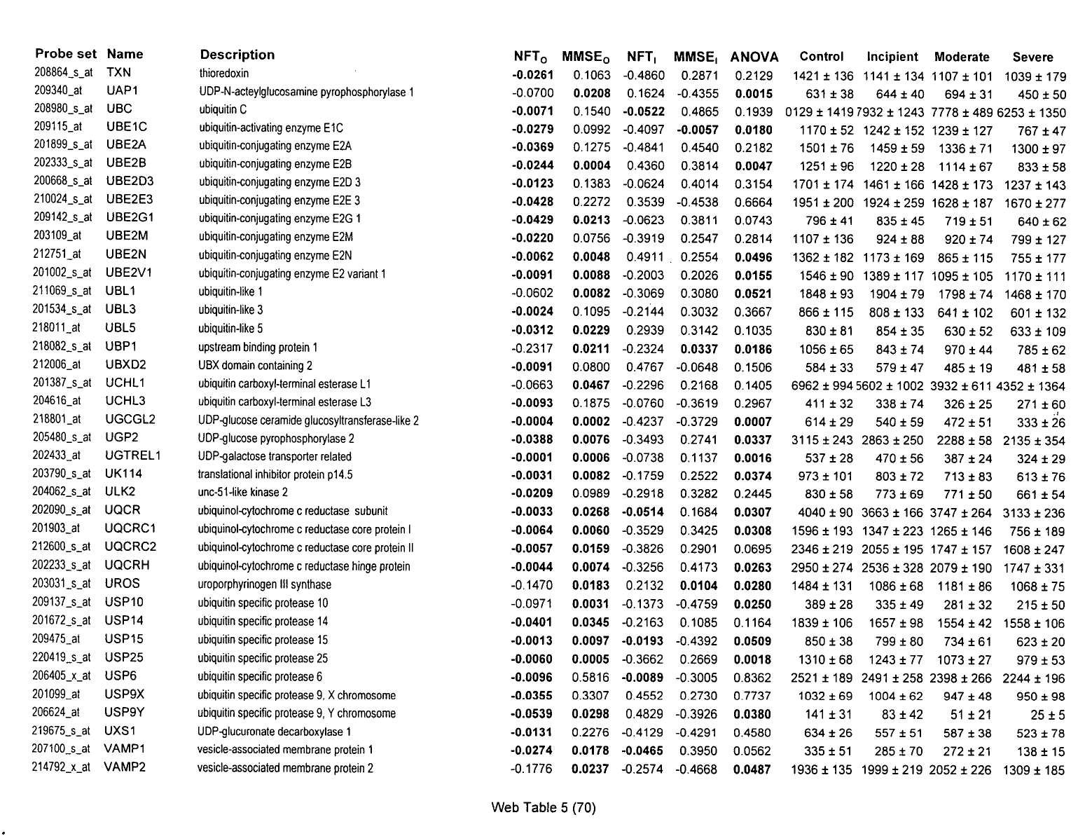

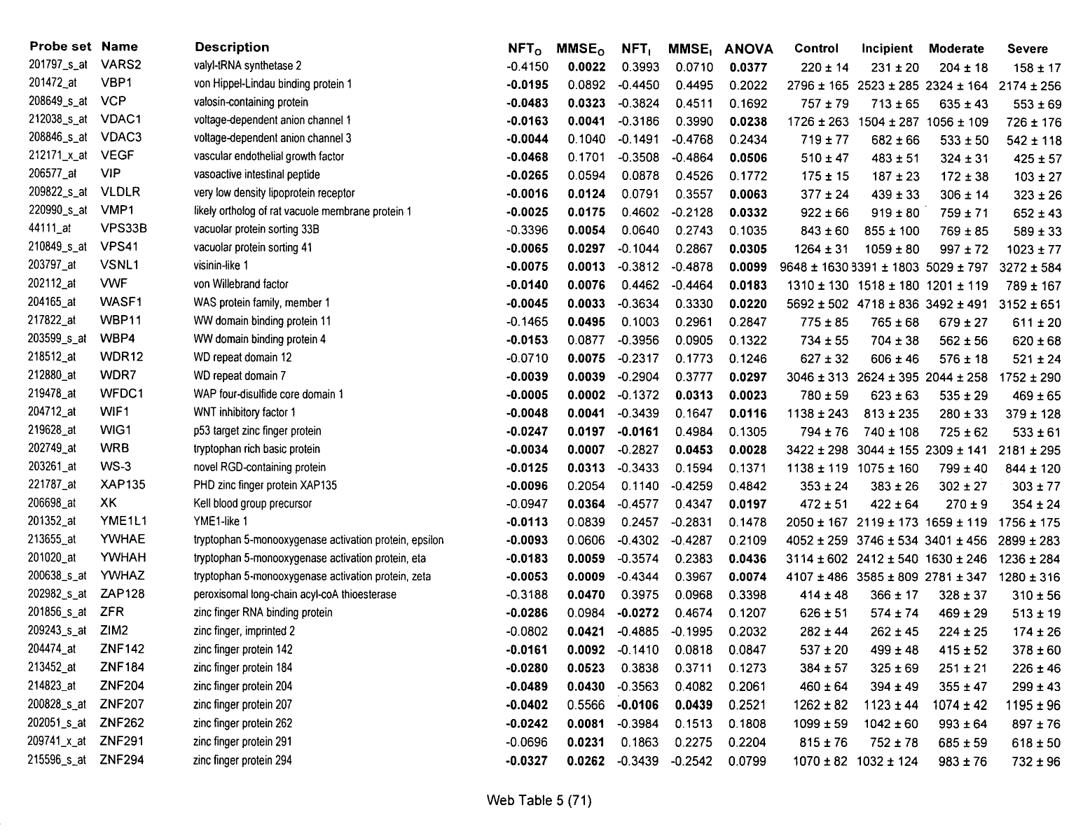

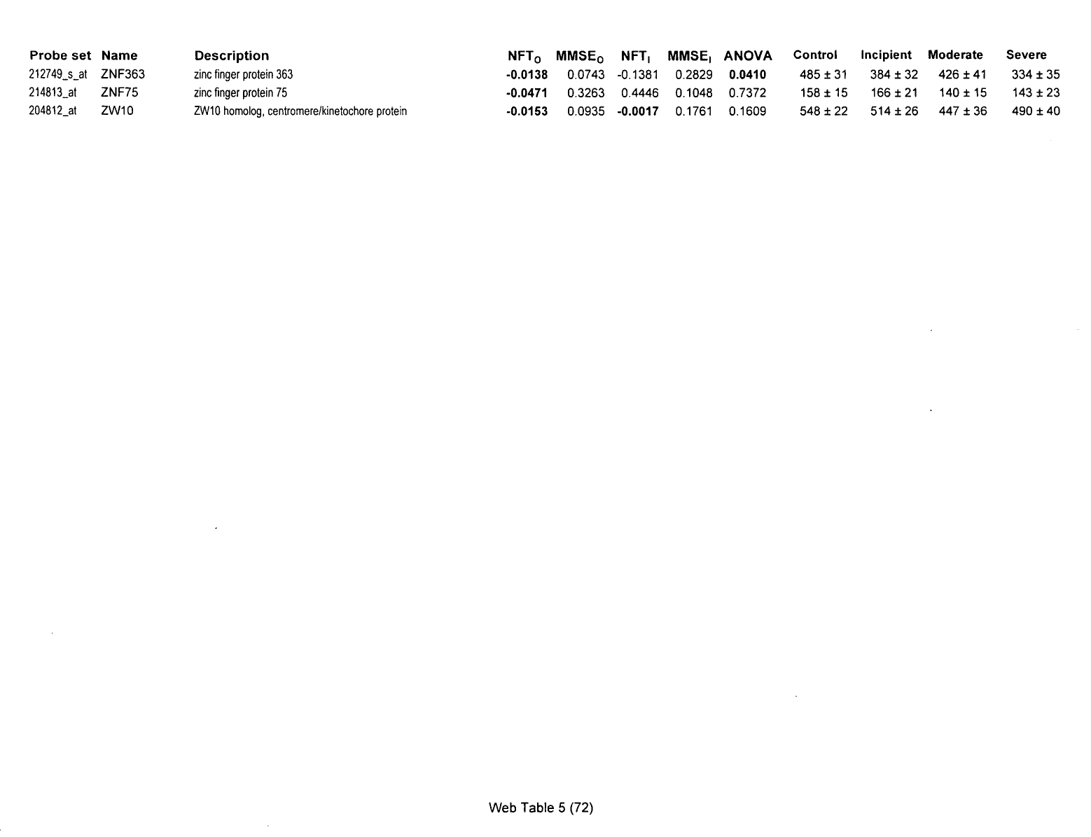

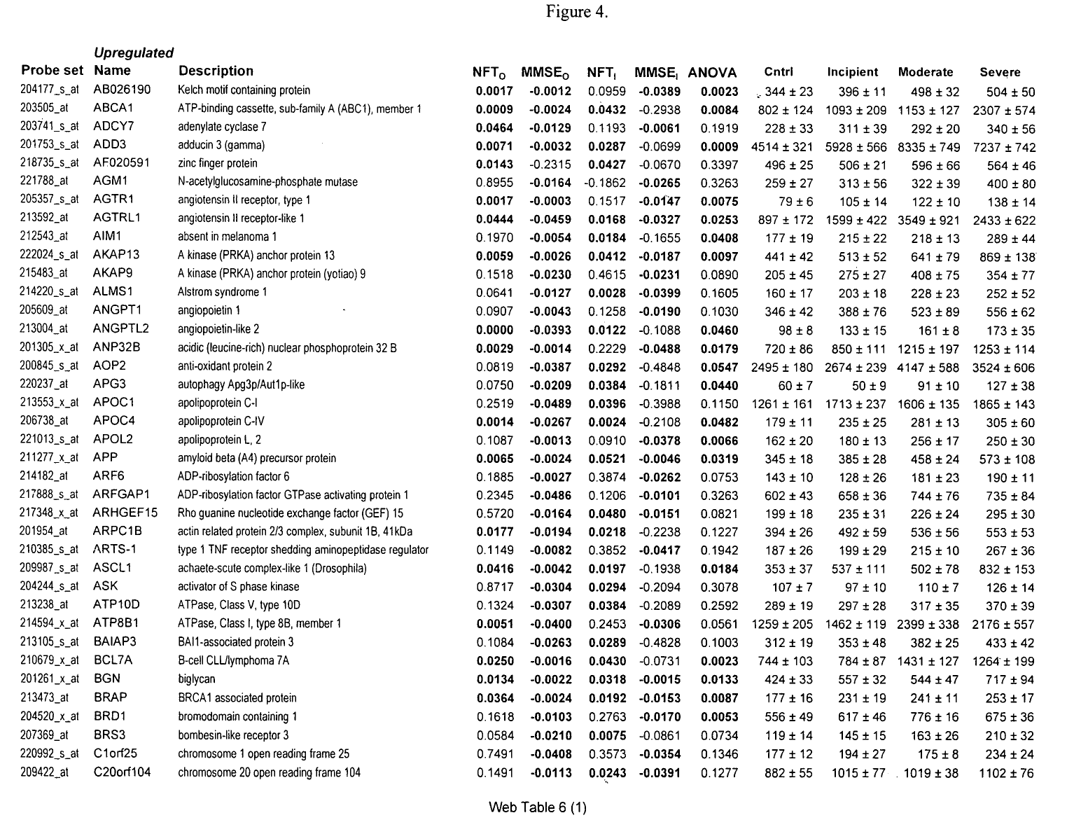

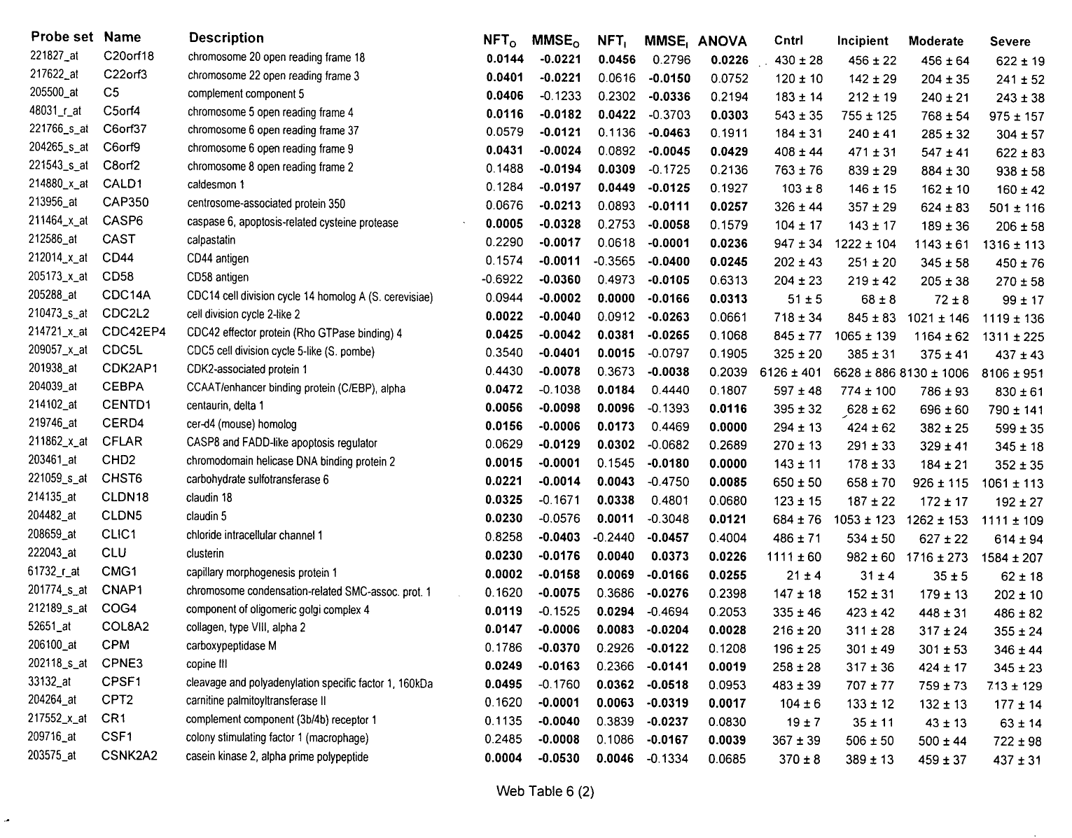

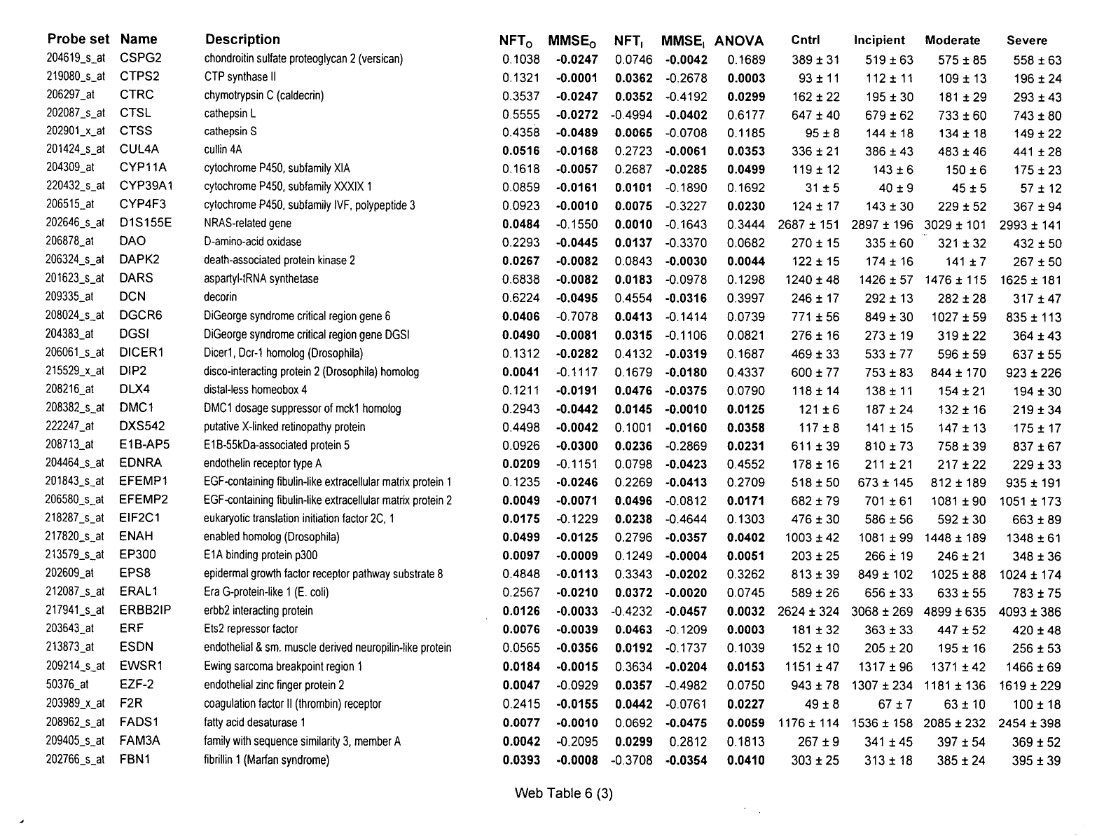

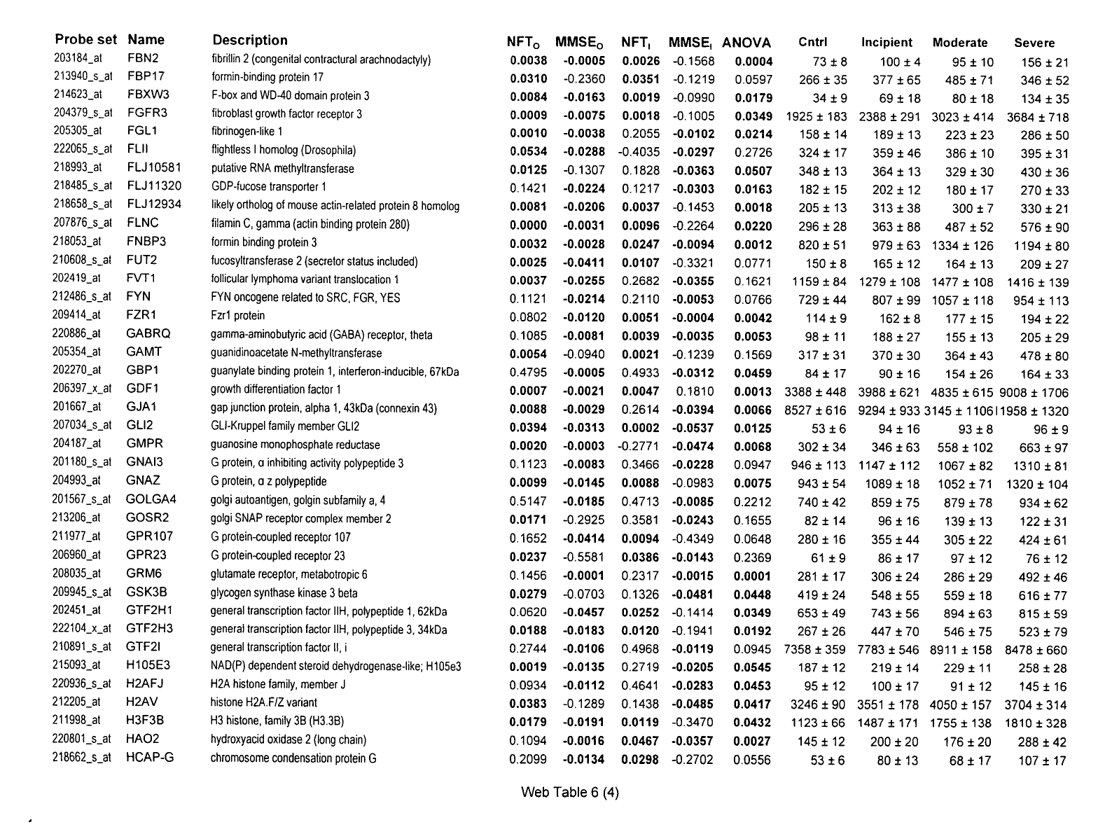

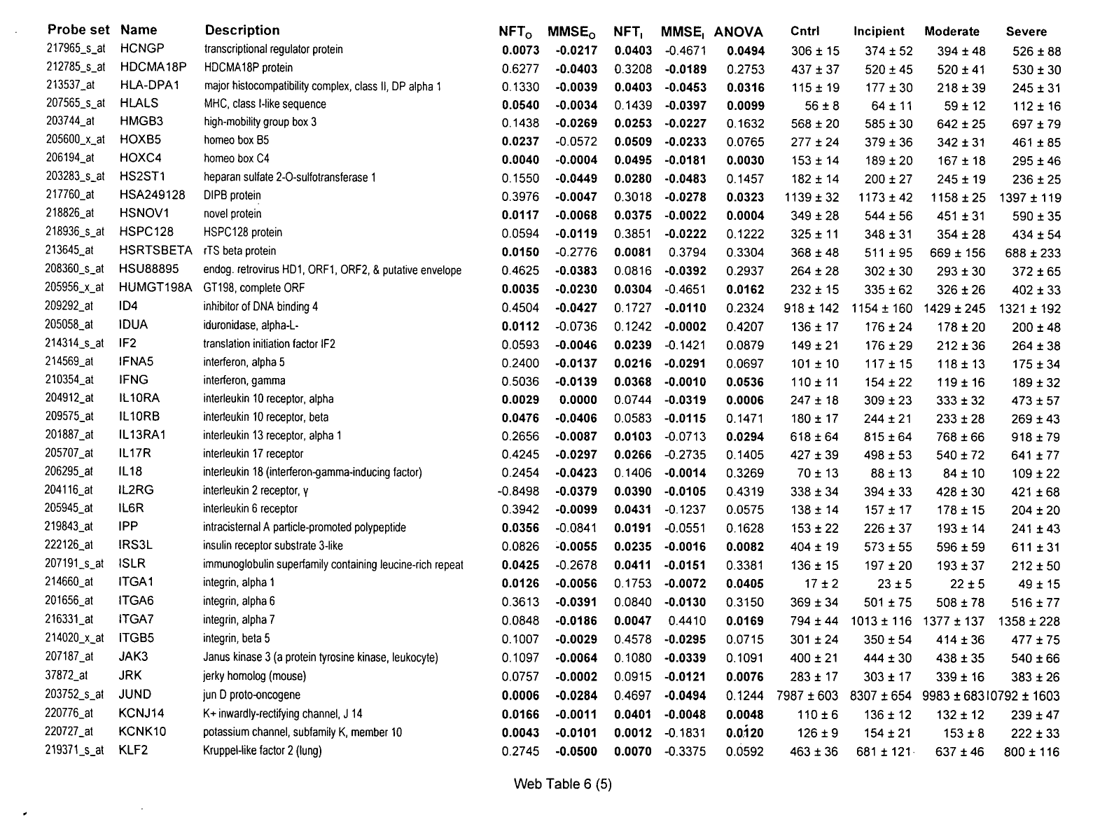

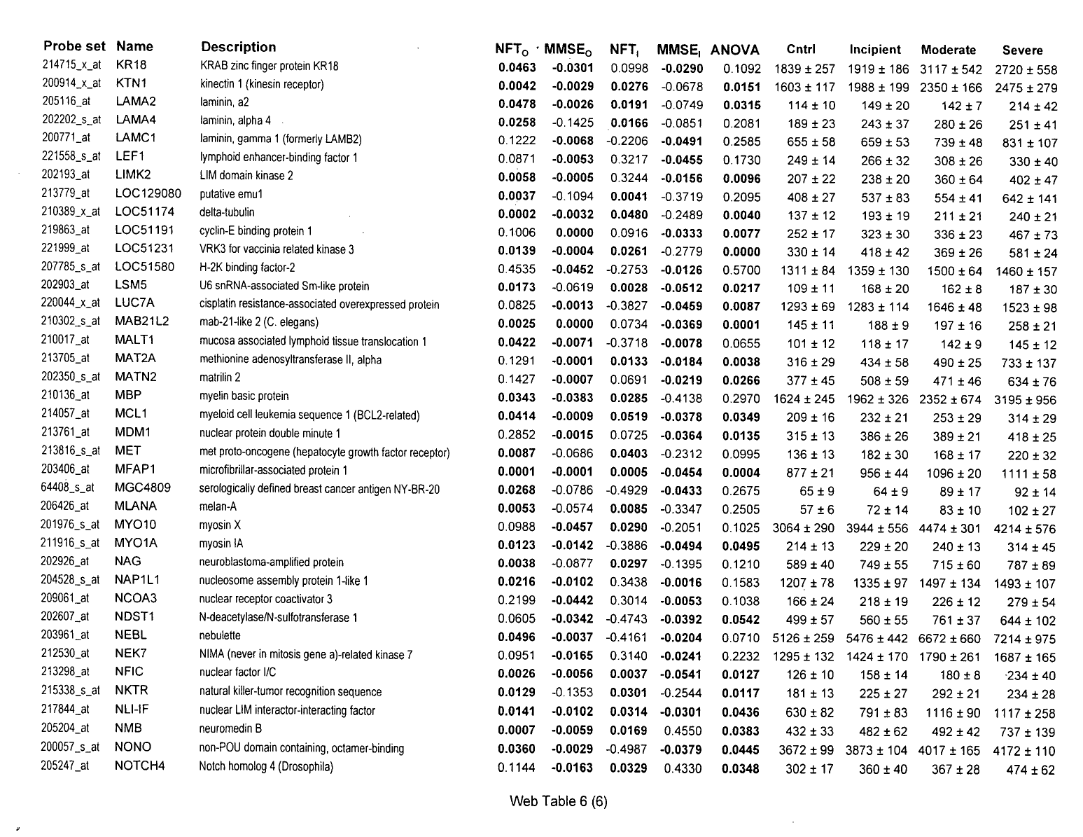

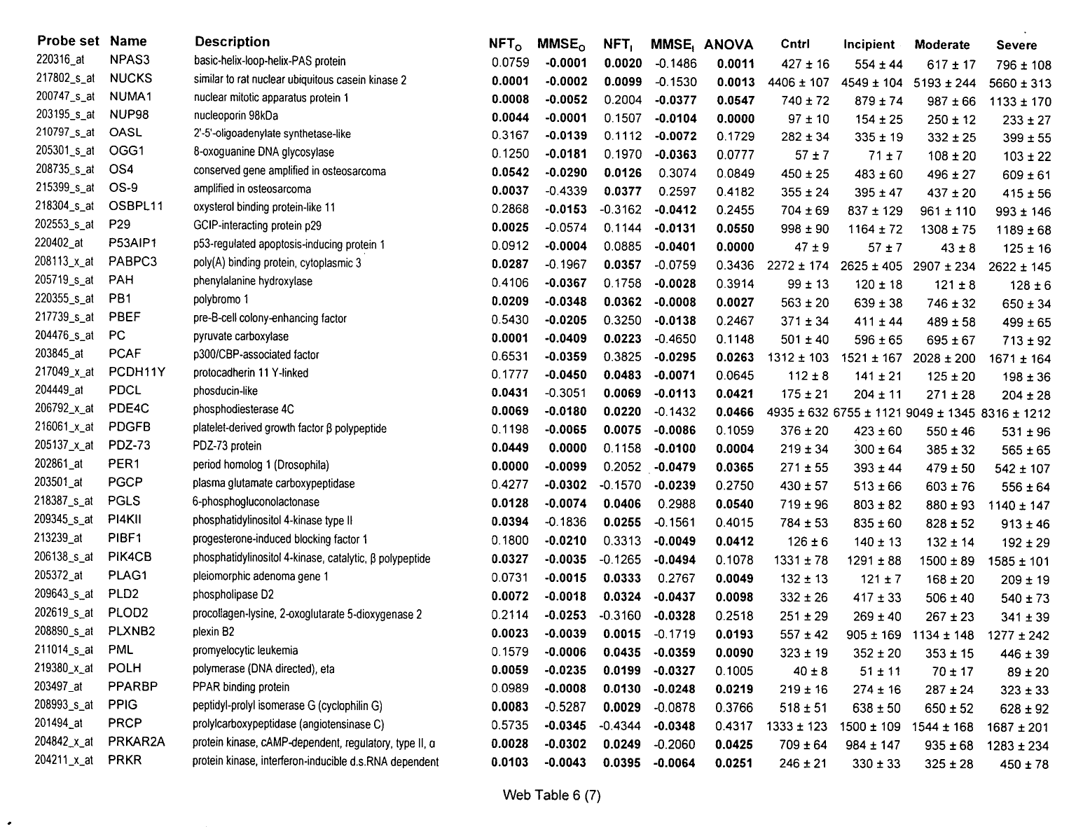

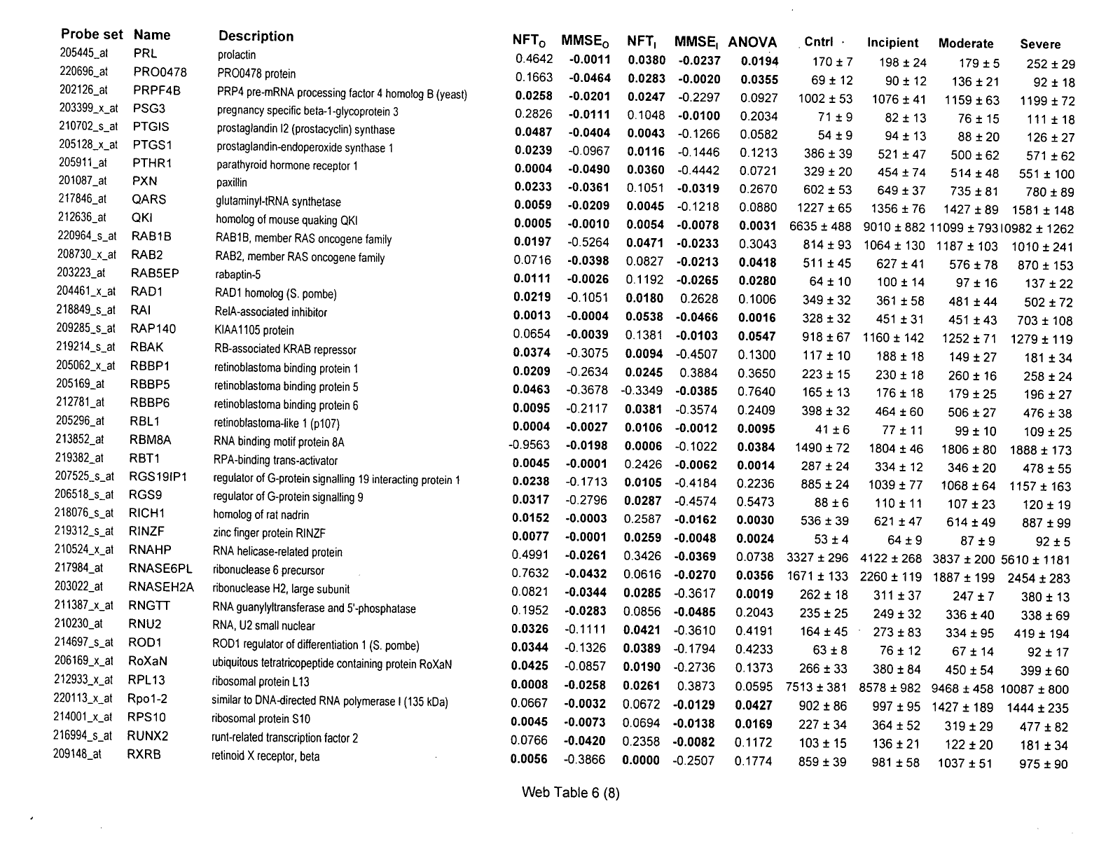

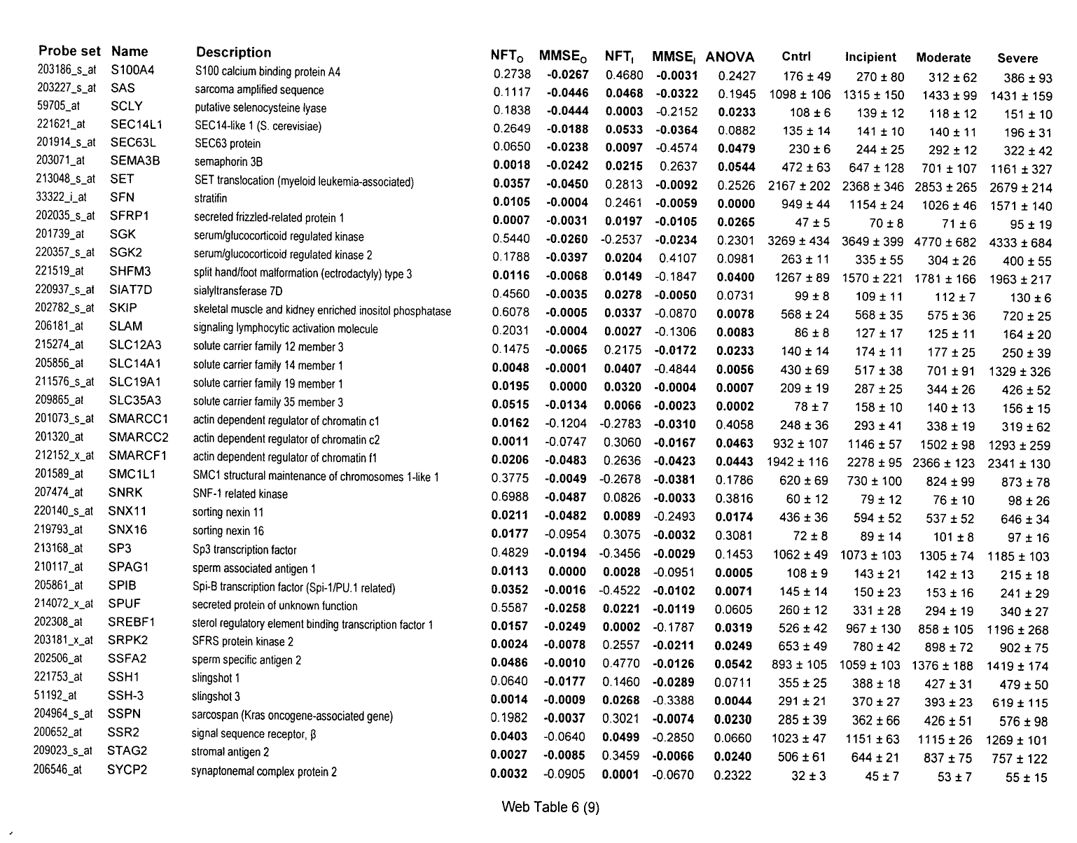

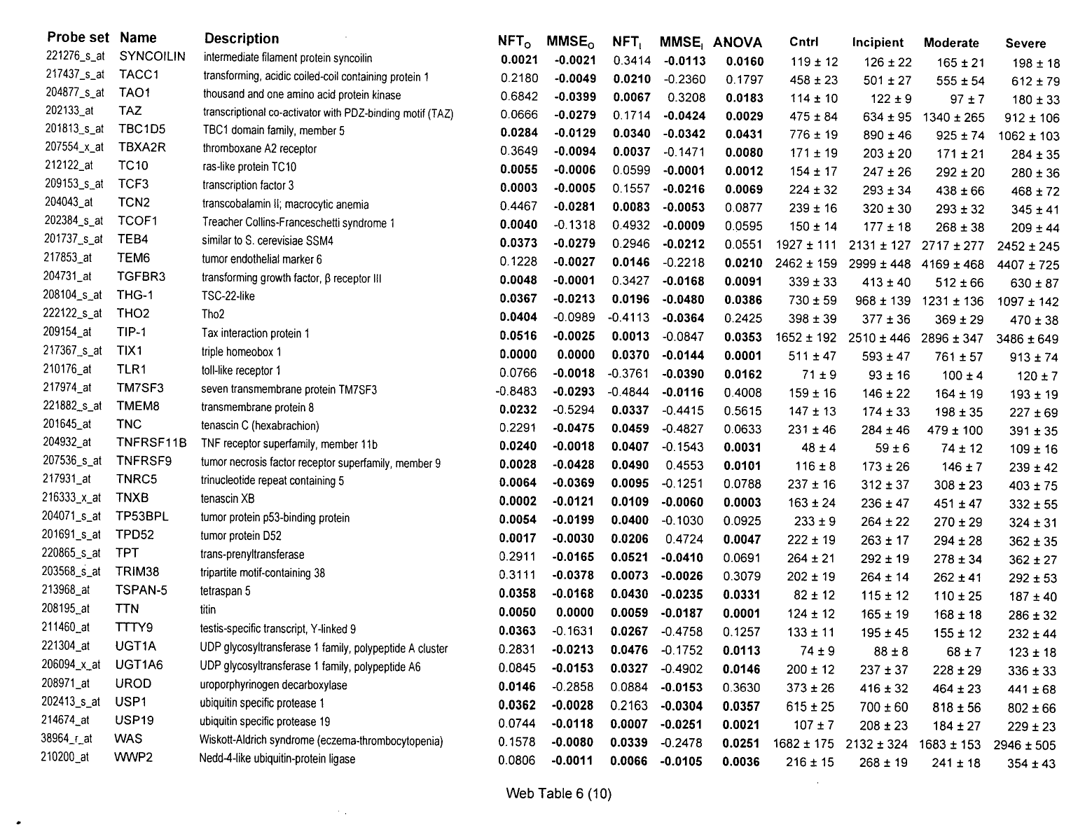

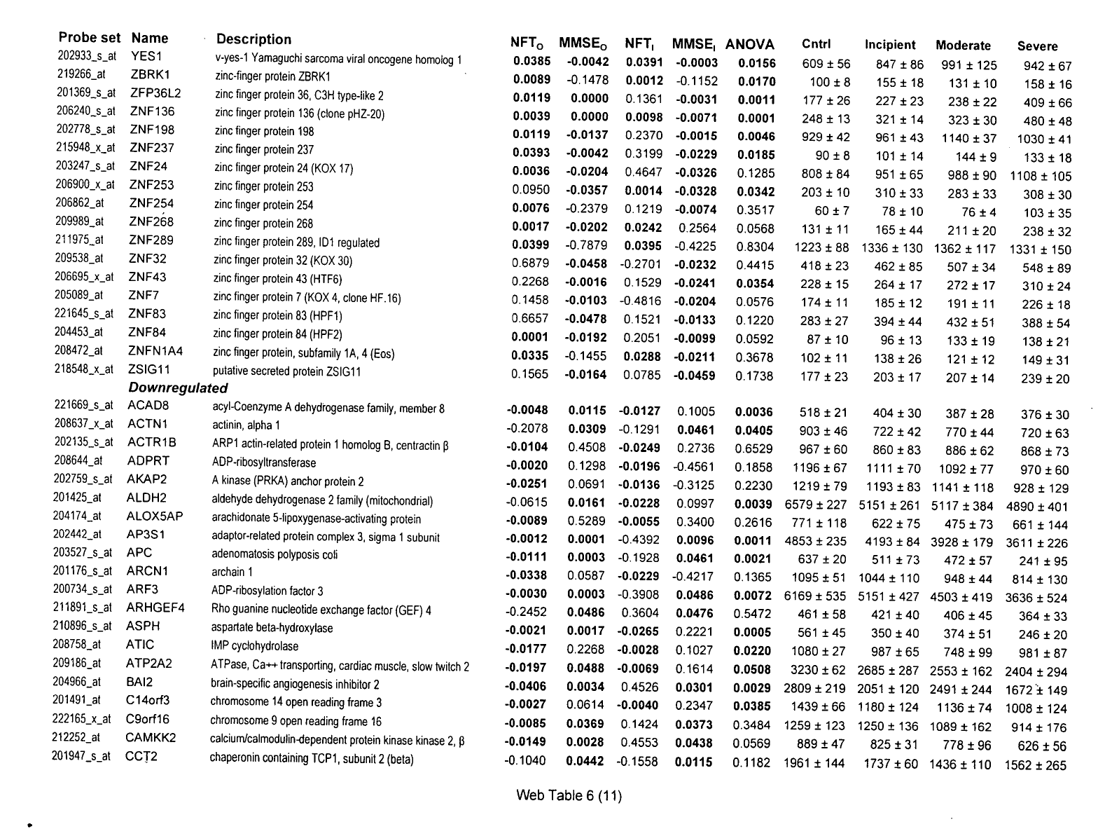

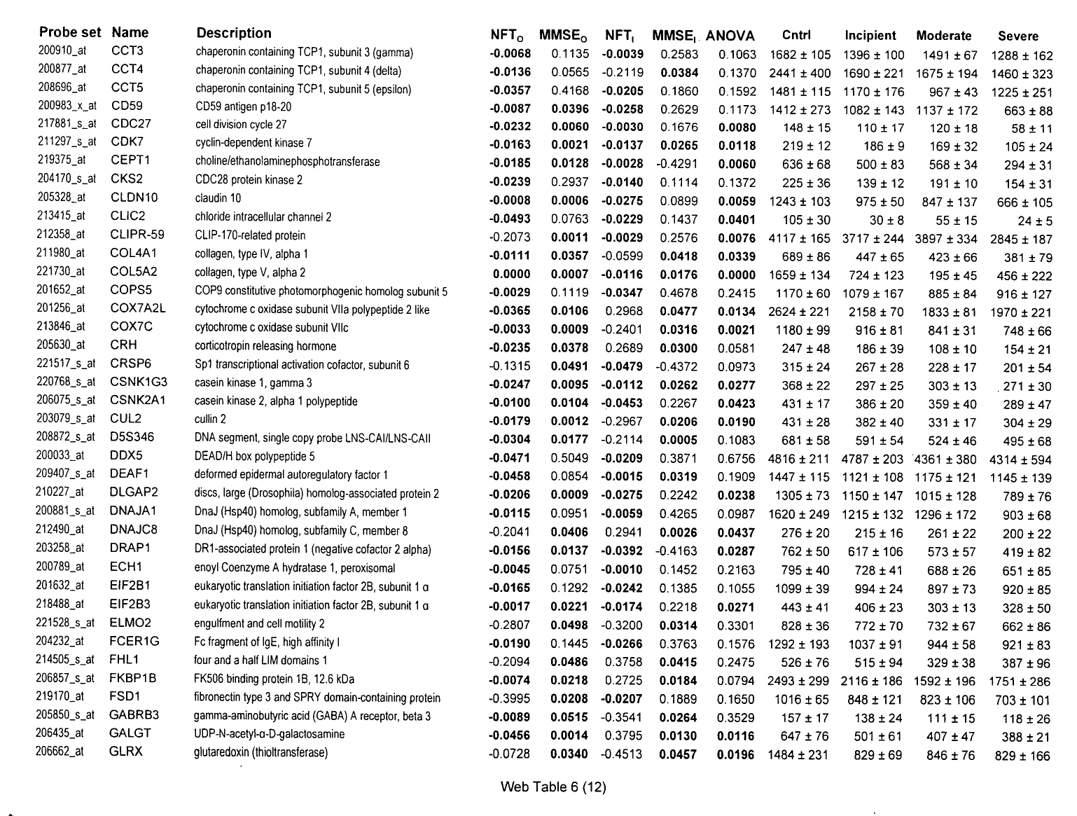

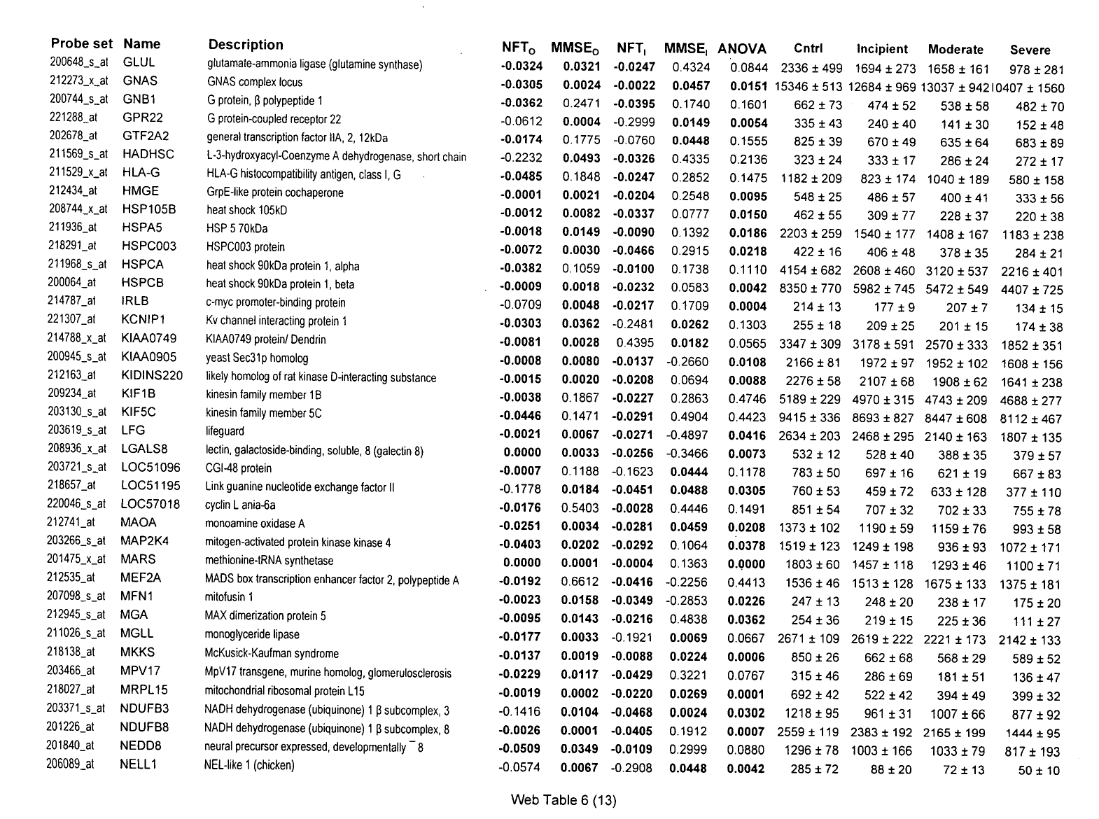

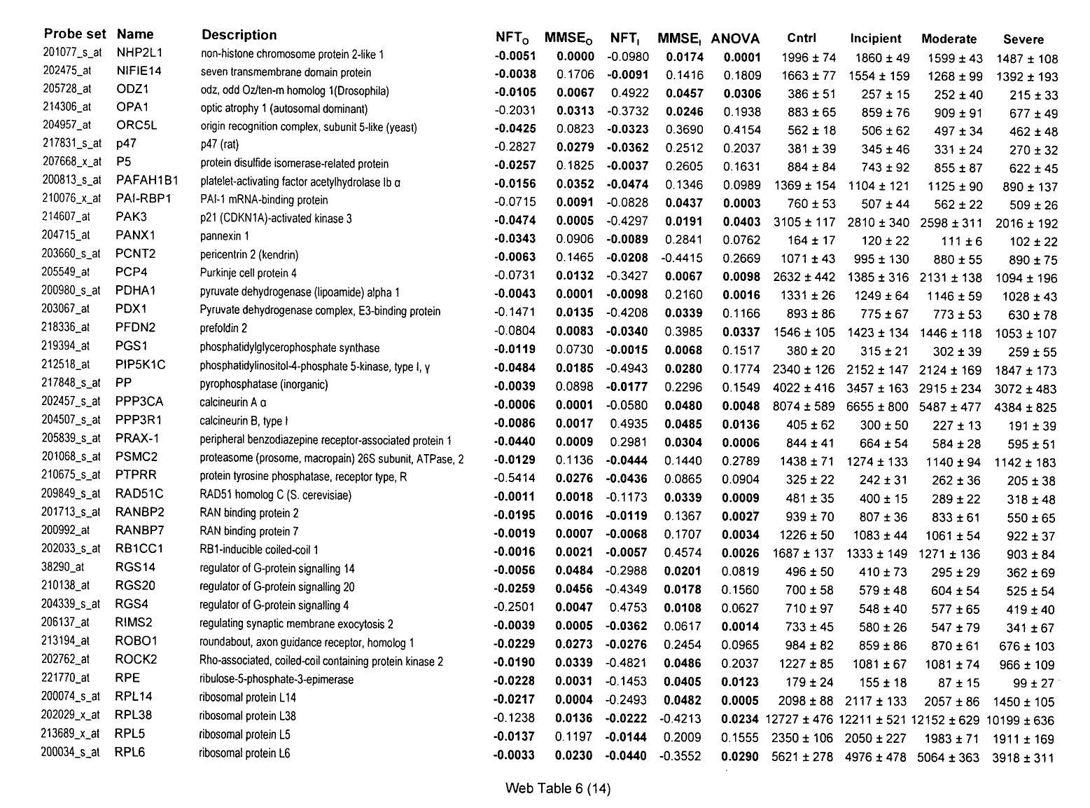

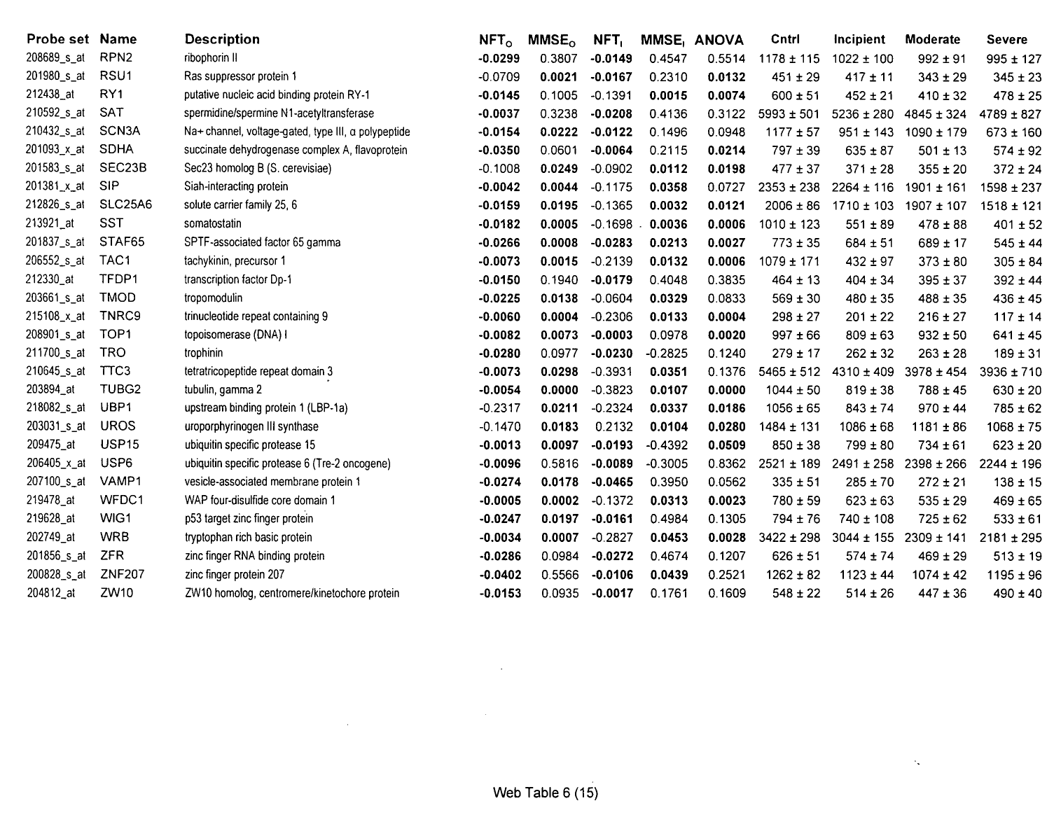

Gene Identification Algorithm (FIG. 1).

To test thousands of genes for correlation with AD markers, while still managing multiple comparison error, all “absent” or undefined (expressed sequence tags) genes (FIG. I A and B) were excluded, thereby reducing expected false positives. Pearson's test was then used to test each of the 9,921 remaining genes for its correlation with MMSE and NFT scores (FIG. IC). A total of 3,413 genes were significantly associated (at P values of ≦0.05) with the MMSE, NET, or both, across all 31 subjects (overall correlations). These correlated genes were termed “AD-related genes” (ADGs).

For both the MMSE and NFT analyses, the false discovery rate, i.e., number of false positives expected because of multiple comparisons divided by the total positives found, was calculated. The false discovery rate provides a worst-case probability that any gene identified (e.g., at P<0.05) by correlation is significant because of the error from multiple testing. The observed false discovery rates (⁻0.20; FIG. 1) are reasonably low for a microarray study, in particular, considering the relatively relaxed P value (P≦0.05), indicating good statistical power. (The false discovery rate generally decreases with more stringent P value criteria. However, the confidence lost with a relaxed P value is substantially offset by the increased confidence gained from expanding the overall number of identified genes and strengthening the EASE analysis of co-regulation).

Because NFT scores increase and MMSE scores decrease with AD severity, genes up-regulated with AD could only correlate positively with NFT scores and negatively with the MMSE, whereas genes down-regulated with AD could only correlate positively with the MMSE and negatively with NFT scores. FIG. 2 illustrates examples of the four patterns of correlation that were possible for ADCs. Overall, 1,977 ADCs were up-regulated and 1,436 were down-regulated. More were correlated with the MMSE than with NET scores. The full set of all identified ADCs is included in Table 5, which is published as supporting information on the PNAS web site.

In a subsequent step (FIG. ID), those genes within this large set of ADGs that also correlated with AD markers across a smaller subgroup comprising incipient AD and control subjects (i.e., all subjects with MMSE a 20 and NFT<20) (n=16) were identified post hoc. Within this subset, only genes correlated in the same direction as their overall correlations were considered. Of the 3,413 overall ADGs, 609 were found also to correlate significantly (at P values of sO.05) in the incipient subgroup, 258 with the MMSE, 262 with NFT scores, and 89 with both (termed “Incipient ADCs” or IADGs). More IADGs were up-regulated with AD (431 genes) than were down-regulated (178 genes) (see Table 6, which is published as supporting information on the PNAS web site, for alphabetical lists of all IADGs).

Biological Processes Associated with ADGs and IADGs.

Using EASE analysis, biological process categories that showed a disproportionately high number of co-regulated genes (significant overrepresentation of ADGs or IADGs in those categories) were identified. The Gene Ontology Biological Process categories in which ADGs were overrepresented by EASE score (in general, at P values of ≦0.05) are shown in Table 2. The overrepresented categories for IADGs are shown in Table 3. Because of the reduced number of genes and lower statistical power in this post hoc analysis, however, the significance level for identified categories of IADGs was set at P≦0.15.

Tables 2 and 3 list significant functional categories having a higher ratio of identified genes to all genes tested on an array for association with that category, relative to the ratio of total identified genes in the study to all genes tested on the array for associations with all categories. Association numbers approximate but are not exactly equal to gene numbers in a category. After each category description (in parentheses) is the ratio of associations for that category and the percentage represented by that ratio. The analogous ratios for total identified up-regulated and down-regulated genes are shown in the headings (Total). EASE, modified Fisher's exact test P value; N/M/B, percentage of genes included in category because they were significant by NFT correlation (N), NMSE correlation (M), or both (B). The complete list of identified ADGs is given alphabetically in Table 5.

TABLE 2
Biological process categories overrepresented by IADGs

| Up-regulated (Total: 1,572/6,265; 25.1%) | EASE | N/M/B | Down-regulated (1,126/6,265; 18.0% | EASE | N/M/B |
|---|---|---|---|---|---|
| Regulation of transcription (269/792; 34% | 0.0000 | 21/38/41 | Energy pathways (17/151; 37.7%) | 0.0000 | 15/15/69 |
| Cell proliferation (210/666; 31.5%) | 0.0001 | 23/43/35 | ATP biosynthesis (16/23; 69.6%) | 0.0000 | 18/9/73 |
| Oncogenesis (24/47; 51.1%) | 0.0003 | 21/39/39 | Synaptic transmission (49/143; 34.3%) | 0.0000 | 9/30/61 |
| Protein amino acid phosphorylation (104/310; 33.5%) | 0.0006 | 23/30/47 | Coenzyme biosynthesis (20/40; 50%) | 0.0000 | 15/15/69 |
| Transition metal ion homeostasis (10/16; 62.5%) | 0.0076 | 18/45/36 | Cation transport (60/197; 30.5%) | 0.0000 | 13/18/69 |
| Positive regulation cell proliferation (25/62; 40.3%) | 0.0119 | 18/68/14 | Protein folding (30/86; 34.9%) | 0.0003 | 32/11/57 |
| Chromatic architecture (34/94; 36.2%) | 0.0186 | 25/43/33 | Tricarboxylic acid cycle (12/222; 54.5) | 0.0006 | 27/27/47 |
| Nucleosome assembly (13/27; 48.1%) | 0.0219 | 11/56/33 | Glycolysis (14/29; 48.3%) | 0.0007 | 6/18/76 |
| Histogenesis and organogenesis (22/57; 38.6%) | 0.0319 | 22/17/61 | Neurogenesis (64/244; 26.2%) | 0.0011 | 19/27/53 |
| Cell adhesion 0.0425(94/314; 29.9%0.) | 0.0346 | 19/46/35 | Amino acid catabolism (13/30; 43.3%) | 0.0038 | 33/0/67 |
| Development (235/850; 27.6%) | 0.0425 | 21/42/37 | Ubiquitin-dependent protein catabolism | 0.0043 | 48/13/39 |
| Complement activation, classical (9/18; 50%) | 0.0576 | 10/40/50 | (27/87; 31%) | | |
| Negative regulation cell proliferation(28/83; 33.7%) | 0.0762 | 09/50/41 | Secretion (14/37; 37.8%) | 0.0095 | 03/35/65 |
| Isoprenoid metabolism (6/10; 60%) | 0.0789 | 00/83/17 | Protein transport (66/288; 22.9%) | 0.0245 | 26/25/49 |
| Apoptosis (72/255; 29.5%) | 0.0818 | 13/32/55 | Neurotransmitter metabolism (6/11; 54.5%) | 0.0329 | 17/17/67 |
| Defense response (102/360; 28.3%) | 0.1010 | 15/57/28 | Axon guidance 8/19; 42.1%) | 0.0404 | 27/9/64 |
| Lipid metabolism (82/288; 28.5%) | 0.1250 | 15/47/38 | Calcium ion transport (11/32; 34.4%) | 0.0482 | 7/7/87 |
| | | | Microtubule-based process (20/73; 27.4%) | 0.0538 | 11/21/68 |
| Biological process categories significantly overrepresented by ADGs (P ≦ 005; EASE SCORE) and a few other selected categories are shown. Numerous | | | | | |
| # other similar significant categories are not included to reduce redundancy. Significant functional categories are those with a higher ratio of identified genes to all | | | | | |
| # genes tested on the array for associations with that category, relative | | | | | |
| # to the ratio of total identified genes in the study to all genes tested on the array for associations with all categories. Association numbers approximate but | | | | | |
| # are not exactly equal to gene numbers in a category. After each category description (in parentheses) is the ratio of associations for that category and the | | | | | |
| # percentage represented by that ratio. The analogous ratios for total identified up-regulated and down-regulated genes are shown in the headings (Total). EASE, | | | | | |
| # modified Fisher's exact test P value; N/M/B, percentage of genes included | | | | | |
| # in category because they were significant by NFT correlation (N), MMSE correlation (M), or both (B). (The complete list of ADGs is given alphabetically in Table 5). | | | | | |

TABLE 3
Biological process categories overrepresented by incipient correlations (IADGs)

| Up-regulated (Total: 379/6, 265; 6%) | EASE | N/M/B | Down-regulated (154/6,265; 3%) | EASE | N/M/B |
|---|---|---|---|---|---|
| Regulation of transcription, DNA . . . (64/7881; 8%) | 0.008 | 30/49/21 | Protein folding (13/86; 15% | 0.000 | 71/21/7 |
| Histogenesis and organogenesis (9/57; 16%) | 0.020 | 33/44/22 | Axon cargo transport (3/5; 60%) | 0.006 | 67/33/0 |
| Chromatin assembly/disassembly (8/52; 15%) | 0.035 | 22/78/0 | Synaptic transmission (10/143; 7%) | 0.008 | 33/67/11 |
| Cell profliferation (52/666; 8%) | 0.041 | 30/46/23 | Protein metabolism (46/1,415; 3%) | 0.028 | 64/28/9 |
| Cell adhesion (26/314; 8%) | 0.092 | 36/46/18 | Microtubule-based movement (4/33; 12%) | 0.046 | 50/50/0 |
| Development (61/850; 7%) | 0.103 | 38/43/19 | Electron transport (10/200; 5%) | 0.055 | 45/36/18 |
| Protein amino acid phosphorylation (25/310; 8%) | 0.122 | 38/46/15 | Cytokinesis (5/61; 8%) | 0.061 | 60/20/20 |
| Cell motility (18/182; 9%) | 0.134 | 35/41/24 | Intracellular transport (15/369; 4%) | 0.066 | 58/37/5 |
| Lipid metabolism (23/288; 8%) | 0.148 | 48/39/13 | GPCR signaling pathway (11/264; 4%) | 0.111 | 33/53/13 |
| Apoptosis (20/244; 8%) | 0.150 | 24/62/14 | Cell surface signal transduction (17/492; 4%) | 0.145 | 43/48/10 |
| Biological process categories significantly overrepresented by IADGs (P ≦ 0.15; EASE SCORE) and a few other selected categories are shown. | | | | | |
| # Numerous other similar significant categories are not included to reduce redundancy. Significant functional categories are those with a higher | | | | | |
| # ratio of identified genes to all genes tested on the array for associations with that category, relative to the ratio of total | | | | | |
| # identified genes in the study to all genes tested on the array for associations with all categories. The association numbers approximate but | | | | | |
| # are not exactly equal to gene numbers in a category. After each category description (in parentheses) is the ratio of associations for that category | | | | | |
| # and the percentage represented by that ratio. The analogous ratios for total identified up-regulated | | | | | |
| # and down-regulated genes are shown in the headings (Total). EASE, modified Fisher's exact test P value; N/M/B, percentage of genes included in | | | | | |
| # category because they were significant by NFT correlation (N), MMSE correlation (M), or both (B). (The complete list of IADGs is given alphabetically in Table 6). | | | | | |

Although many overrepresented categories were similar between Tables 2 and 3, notable differences also occurred. The categories shown in Table 3 were of particular interest because they reflect groups of genes correlated with AD markers in the incipient subjects. Transcription factor, proliferation, and development processes were among the largest categories of up-regulated IADGs. In addition, extracellular matrix/cell adhesion/motility processes, comprising multiple laminins (A2,4), integrins (A1,6,7), tenascins, collagens, cadherins, proteoglycans, and amyloid precursor protein were up-regulated. Of note, several individual members of the semaphorin/plexin pathway, which inhibits axonal elongation, also were up-regulated ADGs (e.g., SEMA3B and plexin 132) (Table 6). Further, histogenesis, apoptosis, phosphorylation, and lipid metabolism, including prostaglandin synthesis, were overrepresented by up-regulated IADGs (Table 3). Although their categories were not overrepresented, several up-regulated IADGs reflected inflammatory and oxidative stress processes (e.g., IFN-gamma, IL-18, interleukin receptors, and AOP2) (Table 6).

For down-regulated categories, a major difference was seen between ADGs and IADGs, in that multiple protein metabolism categories, including folding and transport (immunophilins, chaperones, and heat shock proteins), were overrepresented by IADGs (Table 3), but not ADGs (Table 2). One of the hallmarks of AD, reduced energy metabolism, which dominated the down-regulated categories of ADGs (Table 2), was only reflected in one category, electron transport, of down-regulated IADGs (Table 3).

Calcium Signaling Regulation.

Altered Ca²⁺ signaling is suspected of a role in AD and brain aging and also was identified in a recent microarray study of aging (Blalock et al., supra). Although signaling pathways in general, including Ca²⁺ pathways and transport systems, were down-regulated in AD (Tables 2 and 3), some individual up-regulated Ca²⁺-dependent IADGs included the CAMP response element-binding protein (CREB) cofactor (EP300), a calpain inhibitor (calpastatin), S100A4, and the Ca²⁺-dependent death-associated protein kinase (DAPK2) (Table 6).

Transcription Factors (TFs).

The TF category was the most significantly overrepresented by up-regulated IADGs and ADGs. Table 4 shows the TF-category IADGs correlated with NFT, MMSE scores, or both (only those correlated at P values of ≦0.025 are shown). Review of the functions of the identified TFs revealed that a disproportionately high number are tumor suppressors (TSs) or TS cofactors (boldface), including several of the retinoblastoma (RB) family (also see Table 6 for additional RB members). Many other identified TFs are related to lipid/cholesterol biosynthesis and adipocyte differentiation (underlined) Numerous ;zinc finger TFs favoring transcriptional repression also were identified. Paradoxically, however, a considerable number of the remaining TFs are associated with growth or proliferation. In general, more up-regulated TFs for TS and lipogenesis were correlated with NFT scores than with MMSE, whereas more growth-related TFs were correlated with MMSE (Table 4; see Table 6 for gene descriptions).

TABLE 4
Up-regulated IADGs categorized as TFs

| | | | | |
|---|---|---|---|---|
| | | +NFT | | |
| ANF253 | CEBPA | RBAK | THG-1 | KLF2 |
| SREBF1 | NF1-C | PML | ZNF268 | RBL1(p107)* |
| CS0orf104 | RBBP1 | GL12 | ZBRK1 | PPARBP* |
| RXRB | CERD4 | ASCL1 | GTF21 | |
| | | −MMSE | | |
| SMARCC2 | RUNX2 | ZNF198 | SP18 | SP3 |
| BRD1 | TIX1 | CHD2 | HMGB3 | ENSR1 |
| ANF32 | LOC51580 | HOXB5 | HOXC4 | Rpol-2 |
| ZNF7 | C22orf | NCOA3 | TCF3 | PRKR |
| ZNF43 | ID4 | EP300 | PB1 | ZNF136* |
| ZNF254 | ZNF237 | ZNF83 | ZNF84 | |

Gene symbols for TF IADGs positively correlated with NFT, negatively correlated with MMSE, or both (*) are shown separately (only those with P ≦ 0.025). IADGs for TS (boldface) or lipogenic (underlined) functions are high-lighted. (Full descriptions of all IADGs, alphabetically listed, are available in Table 6).
*TF category IADGs correlated with both NFT and MMSE scores.

Tumor Suppressors (TS).

The high proportion of TS-related TFs prompted us to inspect other biological process categories for genes with TS functions. Many IADGs with TS or cellular differentiation functions were found in the phosphorylation, apoptotic, cell cycle, and other categories (e.g., TGF-β, GSK3B, PDCD4, FZR 1, SFRP1, AIMI, DAPK2, and CDK2AP1). Conversely, inspection of the down-regulated IF categories (not shown) revealed many TFs important for growth and proliferation, including several of the MYC family (MGA and IRLB) and DPI(TFDP 1), a member of the growth-promoting E2F family targeted by the RB family of TSs (Table 6).

PKA Pathways.

The cAMP-dependent protein kinase (PKA) pathway stimulates growth in some cell types and differentiation and inhibition of growth in others. Several PKA-related genes were up-regulated IADGs, including A kinase-anchoring molecules (AKAP9, AKAP13, and CAP350), adenylate cyclase 7, and the PKA Type RII α regulatory subunit (Table 6).

### EXAMPLE 2

Diagnosis of AD or IAD in a Patient.

Total RNA is isolated from a neural tissue sample or tissue sample obtained from the patient using the TRIzol reagent and following the manufacturer's RNA isolation protocol (Invitrogen, #15596). For tissue samples, e.g., brain tissue, one milliliter of TRIzol solution is added to each tube containing the frozen tissue block, and the tissue is homogenized by ten passages through an 18.5 gauge syringe needle. After centrifugation, the RNA is precipitated from the aqueous layer, washed, and dissolved in RNase-free water. RNA concentration and integrity are assessed by spectrophotometry and gel electrophoresis. The RNA samples may be stored at −80° C. until use.

Gene expression analyses are performed using the Affymetrix GeneChip System. Gene chips can be custom made to contain oligonucleotides specific to only a subset of human genes, such as those AD/IAD correlated genes listed in Table 5, or Table 6, or subsets of these genes. Subsets of these genes include genes identified in general in Tables 2, 3, and specifically in 4, and may comprise any combination of the genes identified in these tables. The gene chips may be made in any format to accommodate content requirements, ranging from 520 to over 61,000 sequences per array. Duplicates of the oligonucleotide sequences may be run on the same or on separate arrays as controls.

The labeling of RNA samples, GeneChip (HG-U133A) hybridization, and array scanning are performed according to the Affymetrix GeneChip Expression Analysis Manual (Version 5, (2000)). For brain tissue, each CA1 subfield RNA is processed and run on a separate gene chip. For other tissue samples duplicate chips may be made and tested. Briefly, about 20 μg of cRNA is applied to one chip. The hybridization ⁱs run overnight in a rotating oven at about 45° C. The chips are then washed and stained on a fluidics station and scanned at a resolution of about 3 μm in a confocal scanner (e.g., AgilentAffymetrix GeneArray Scanner).

Microarray suite software (MAS 5.0, Affymetrix) is used to calculate the overall noise of the image (Qraw).

The algorithms used to determine average difference expression (ADE) scores (expression level) and presence/absence calls are described in the Microarray Suite 5.0 Manual and form the basis for determining expression (relative abundance) of transcripts and whether a particular transcript is reliably detectable, respectively.

### EXAMPLE 3

Drug Screening

Compounds are tested as potential therapeutic agents for AD by administering a test compound to an animal exhibiting all or some of the symptoms of AD. Various animal models can be used to analyze effects of test compounds on the expression of ADGs and/or IADGs, as described above. Preferred are animals such as mice that exhibit characteristics associated with the pathophysiology of AD. Administration of the test compound in a pharmaceutically effective carrier and via an administrative route that reaches the target tissue in an appropriate therapeutic amount is preferred.

Analysis of ADG and/or IADG expression in the brain tissue of the mice after exposure to the test compound in comparison to expression observed in control animals is a preferred method of determining the effect of the test compound on genes and/or biological pathways associated with AD.

What is claimed:

1. An oligonucleotide array comprising a solid support comprising a plurality of different oligonucleotide probes, each oligonucleotide probe specific for a gene listed in Table 6.

2. The oligonucleotide array according to claim 1 wherein each oligonucleotide probe is specific for a gene listed in Table 4.

3. The oligonucleotide array of claim 1 wherein said plurality of oligonucleotide probes comprises probes for genes encoding human transcription factors, proliferation-associated protein, tumor suppressor proteins, histogenesis-associated protein, apoptosis-associated protein, phosphorylation enzymes, lipid metabolism-associated proteins, extracellular matrix, cell adhesion protein, motility protein, laminin, integrin, tenascin, collagen, cadherin, proteoglycan, SEM3AB, plexin B2, and combinations thereof.

4. A method of detecting in a brain tissue or neural tissue sample an alteration in the expression pattern of a plurality of genes correlated with incipient Alzheimer's disease (IAD) relative to expression of said plurality of genes in a control comprising
a) obtaining RNA from said sample;
b) contacting said RNA with an array according to claim 1 under conditions that permit hybridization of said RNA to oligonucleotides covalently attached to said array; and
c) detecting the presence or absence of said alteration in the expression pattern of said plurality of genes correlated with IAD relative to expression of said plurality of genes in a control.

5. The method according to claim 4 wherein the sample is a brain sample.

6. The method of claim 4 wherein the sample is a neural tissue sample.

7. The method of claim 4 wherein the array comprises oligonucleotides selected from the group of probes listed in Table 4.

8. A method for diagnosing AD in a patient comprising
a) obtaining a brain tissue or neural tissue sample from the patient and extracting RNA there from;
b) contacting said RNA with an array according to claim 1 under conditions that permit hybridization of said RNA to oligonucleotides covalently attached to said array;
c) detecting the presence or absence of an alteration in the expression pattern of a plurality of genes correlated with incipient Alzheimer's disease (IAD) relative to expression of said plurality of genes in a control; and
d) correlating the presence of an alteration in the expression pattern of said plurality of genes correlated with IAD relative to expression of said plurality of genes in a control to the presence of AD.

9. The method of claim 8 further comprising administering a mini mental state examination (MMSE) or neurological test for AD or IAD to the patient and correlating the results with the presence or absence of AD.

10. The method of claim 8 wherein the sample is a brain sample.

11. The method of claim 8 wherein the sample is a neural tissue sample.

12. The method of claim 8 wherein the array comprises oligonucleotide probes selected from the group of probes listed in Table 4.

13. A method of screening a test compound for treatment of AD or IAD comprising,
a) administering the test compound to an animal or human exhibiting all or some of the symptoms of AD;
b) obtaining a brain tissue or neural tissue sample from the animal or human and obtaining RNA there from;
c) contacting said RNA with an array according to claim 1 under conditions that permit hybridization of said RNA to oligonucleotide probes covalently attached to said array; and
d) detecting the presence or absence of an alteration in the expression pattern of a plurality of genes correlated with IAD relative to expression of said plurality of genes in an untreated control animal or human exhibiting all or some of the symptoms of AD.

14. A kit comprising an array according to claim 1 and at least one reagent.

15. The kit according to claim 14 wherein the array comprises oligonucleotide probes selected from the group of probes listed in Table 4.

16. A method of detecting an alteration in the expression pattern of a plurality of proteins encoded by genes correlated with incipient Alzheimer's disease (IAD) relative to expression of said plurality of proteins in a control in a brain tissue, neural tissue or spinal fluid sample of an animal, said method comprising
a) measuring the relative amount of individual proteins in said sample, wherein each of said proteins is encoded by a gene correlated with IAD; and
b) correlating an increase or decrease in the amount of a plurality of said proteins relative to amount of said plurality of proteins to an alteration in the expression pattern of the plurality of genes encoding said proteins.

17. The method of claim 16 wherein the plurality of genes is selected from the group of genes listed in Table 6.

18. The method of claim 16 wherein the plurality of genes is selected from the group of genes listed in Table 4.

19. A method for diagnosing AD in a patient comprising
a) obtaining a brain tissue, neural tissue or spinal fluid sample from the patient and extracting protein there from;
b) measuring the relative amount of individual proteins in said sample, wherein each of said proteins is encoded by a gene correlated with IAD; and
c) correlating an increase or decrease in the amount of a plurality of said proteins relative to amount of said plurality of proteins to the presence of AD in said patient.

20. The method of claim 19 wherein each of the plurality of proteins is encoded by a gene listed in Table 6.
